(19) United States
(12) Patent Application Publication
(10) Pub. No.: US 2023/0310418 A1
(43) Pub. Date: Oct. 5, 2023

(54) COMPOSITIONS AND METHODS FOR TREATING CANCER OR PREVENTING, INHIBITING OR REDUCING RISK OF METASTASIS OF A CANCER

(71) Applicants: Lixte Biotechnology, Inc., Pasadena, CA (US); STICHTING HET NEDERLANDS KANKER INSTITUUT - ANTONI VAN LEEUWENHOEK ZIEKENHUIS, Amsterdam (NL)

(72) Inventors: John S. KOVACH, Pasadena, CA (US); Rene BERNARDS, Amsterdam (NL); Matheus Henrique DIAS, Amsterdam (NL)

(21) Appl. No.: 18/092,606

(22) Filed: Jan. 3, 2023

Related U.S. Application Data
(60) Provisional application No. 63/329,314, filed on Apr. 8, 2022.
(60) Provisional application No. 63/296,351, filed on Jan. 4, 2022.

(51) Int. Cl. A61K 31/496; A61K 31/519; A61K 31/4725; A61P 35/00

(52) U.S. Cl. CPC A61K 31/496; A61K 31/4725; A61K 31/519; A61P 35/00

(57) ABSTRACT

This invention provides compositions of (a) LB-100 or an LB-100 ester, or a pharmaceutically acceptable salt thereof, and (b) a WEE1 kinase inhibitor, checkpoint kinase 1 (“CHK1”) inhibitor, B-cell lymphoma-extra large (“BCL-xL”) inhibitor, BCL-xL proteolysis-targeting chimera (“BCL-xL PROTAC”), pan-BCL inhibitor or ataxia telangiectasia and Rad3-related (“ATR”) inhibitor. This invention further provides methods for treating cancer or for preventing, inhibiting, or reducing risk of metastasis of a cancer, such as colorectal cancer, cholangiocarcinoma, pancreatic cancer or ovarian cancer, using (a) LB-100 or an LB-100 ester, or a pharmaceutically acceptable salt thereof, and (b) a WEE1 kinase inhibitor, CHK1 inhibitor, BCL-xL inhibitor, BCL-xL PROTAC, pan-BCL inhibitor or ATR inhibitor.

### CROSS-REFERENCE TO RELATED APPLICATIONS

The present application claims the benefit of U.S. Provisional Application No. 63/296,351, filed Jan. 4, 2022, and U.S. Provisional Application No. 63/329,314, filed Apr. 8, 2022, each of which is incorporated herein by reference in its entirety.

### BACKGROUND OF THE INVENTION

A ubiquitous feature of cancer cells is their increased mobilization of stress response pathways, as many of these pathways may have to be mobilized by the cancer cells to counterbalance the oncogenic activity and support the tumorigenic state. This overall scenario suggests that a “counter-intuitive” deliberate activation of mitogenic signaling may not only disrupt the homeostasis of cancer cells, but also sensitize them to drugs targeting the stress-coping pathways that are frequently activated in these cells.

While the arsenal of compounds developed to restrain mitogenic signaling in cancer cells is vast, hyperactivation of these pathways for therapeutic purposes is generally uncharted territory.

The present invention addresses this unmet need.

### SUMMARY OF THE INVENTION

The present invention provides compositions comprising (1) an effective amount of (a) LB-100 or an LB-100 ester, or a pharmaceutically acceptable salt thereof and (b) a WEE1 kinase inhibitor, checkpoint kinase 1 (“CHK1”) inhibitor, B-cell lymphoma-extra large (“BCL-xL”) inhibitor, BCL-xL proteolysis-targeting chimera (“BCL-xL PROTAC”), pan-BCL inhibitor, or ataxia telangiectasia and Rad3-related (“ATR”) inhibitor; and (2) a pharmaceutically acceptable carrier or vehicle (each composition being a “composition of the invention”, each of the WEE1 kinase inhibitor, CHK1 inhibitor, BCL-xL inhibitor, BCL-xL PROTAC, pan-BCL inhibitor, and ATR inhibitor being “another anti-cancer agent”).

The present invention also provides methods for treating cancer, comprising administering to a subject in need thereof an effective amount of: (a) LB-100 or an LB-100 ester, or a pharmaceutically acceptable salt thereof, and (b) a WEE1 kinase inhibitor, CHK1 inhibitor, BCL-xL inhibitor, BCL-xL PROTAC, pan-BCL inhibitor, or ATR inhibitor, wherein the cancer is hepatocellular carcinoma (hepatoma), cholangiocarcinoma, colorectal carcinoma, small cell lung cancer, non-small cell lung cancer, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing’s tumor, leiomyosarcoma, rhabdomyosarcoma, pancreatic cancer, breast cancer, triple negative breast cancer, ovarian cancer, endometrial carcinoma, Fallopian tube cancer, prostate cancer, gastrointestinal stromal tumor (GIST), esophageal cancer, gallbladder cancer, gastrointestinal carcinoid tumor, duodenal cancer, gastroesophageal junction cancer, islet cell cancer, gastric cancer, anal cancer, cancer of the small intestine, pseudomyxoma peritonei, head and neck squamous cell carcinoma, Merkel cell carcinoma, tumor mutational burden-high cancer (TMB-H), microsatellite stable (MSS), mismatch repair proficient colon cancer, thyroid cancer, renal cell carcinoma, choriocarcinoma, seminoma, embryonal carcinoma, Wilms’ tumor, urothelial carcinoma, testicular cancer, bladder carcinoma, glioma, glioblastoma, multiforme, astrocytoma, medulloblastoma, craniopharyngioma, oligodendroglioma, malignant meningioma, diffuse intrinsic pontine glioma, melanoma, neuroblastoma, retinoblastoma, acute lymphoblastic B-cell leukemia, acute lymphoblastic T-cell leukemia, acute myeloblastic leukemia (AML), acute promyelocytic leukemia (APL), acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia (CML), chronic lymphocytic leukemia (CLL), hairy cell leukemia, multiple myeloma, lymphoblastic leukemia, myelogenous leukemia, lymphocytic leukemia, Hodgkin’s lymphoma, non-Hodgkin’s lymphoma, primary mediastinal large B-cell lymphoma, Waldenstrom’s macroglobulinemia, heavy chain disease, or polycythemia vera.

The present invention further provides methods for preventing, inhibiting, or reducing risk of metastasis of a cancer, comprising administering to a subject in need thereof an effective amount of: (a) LB-100 or an LB-100 ester, or a pharmaceutically acceptable salt thereof and (b) a WEE1 kinase inhibitor, CHK1 inhibitor, BCL-xL inhibitor, BCL-xL PROTAC, pan-BCL inhibitor, or ATR inhibitor, wherein the cancer is hepatocellular carcinoma (hepatoma), cholangiocarcinoma, colorectal carcinoma, small cell lung cancer, non-small cell lung cancer, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing’s tumor, leiomyosarcoma, rhabdomyosarcoma, pancreatic cancer, breast cancer, triple negative breast cancer, ovarian cancer, endometrial carcinoma, Fallopian tube cancer, prostate cancer, gastrointestinal stromal tumor (GIST), esophageal cancer, gallbladder cancer, gastrointestinal carcinoid tumor, duodenal cancer, gastroesophageal junction cancer, islet cell cancer, gastric cancer, anal cancer, cancer of the small intestine, pseudomyxoma peritonei, head and neck squamous cell carcinoma, Merkel cell carcinoma, tumor mutational burden-high cancer (TMB-H), microsatellite stable (MSS), mismatch repair proficient colon cancer, thyroid cancer, renal cell carcinoma, choriocarcinoma, seminoma, embryonal carcinoma, Wilms’ tumor, urothelial carcinoma, testicular cancer, bladder carcinoma, glioma, glioblastoma, multiforme, astrocytoma, medulloblastoma, craniopharyngioma, oligodendroglioma, malignant meningioma, diffuse intrinsic pontine glioma, melanoma, neuroblastoma, retinoblastoma, acute lymphoblastic B-cell leukemia, acute lymphoblastic T-cell leukemia, acute myeloblastic leukemia (AML), acute promyelocytic leukemia (APL), acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia (CML), chronic lymphocytic leukemia (CLL), hairy cell leukemia, multiple myeloma, lymphoblastic leukemia, myelogenous leukemia, lymphocytic leukemia, Hodgkin’s lymphoma, non-Hodgkin’s lymphoma, primary mediastinal large B-cell lymphoma, Waldenstrom’s macroglobulinemia, heavy chain disease, or polycythemia vera.

Each of the above methods is a “method of the invention”.

### BRIEF DESCRIPTION OF THE DRAWINGS

FIG. 1A shows the cytotoxicity effects of LB-100 against colorectal cancer cell lines in a short-term cell viability assay, where cells were cultured with increasing concentrations of LB-100 for 5 days, then the cell viability was measured using resazurin.

FIG. 1B shows the cytotoxicity effects of LB-100 against colorectal cancer cell lines in a longer-term cell proliferation assay, where cells were grown in the absence or presence of LB-100 at the indicated concentrations for 7 days, then fixed and stained. FIG. 1C provides Western blots showing that mitogenic signaling and stress pathways are activated in colorectal cancer cell lines (DiFi, HT-29, and SW-480) treated with 4 µM LB-100. FIG. 1D provides Western blots showing that mitogenic signaling and stress pathways are activated in a SW-480 colorectal cancer cell line treated with 2.5 µM LB-100. FIG. 1E provides Western blots showing that mitogenic signaling and stress pathways are activated in DiFi and HT-29 colorectal cancer cell lines treated with 2.5 µM or 5 µM LB-100. Vinculin was used as a loading control in each Western blot analysis.

FIG. 2A shows the stress-focused drug screening method used to evaluate the ability of LB-100 to sensitize cancer cells to stress-targeted drugs. FIG. 2B shows the cytotoxicity results in SW-480 colorectal cancer cells from the stress-focused drug screen of FIG. 2A of 164 drugs in the presence of LB-100 as compared to control. The indicated compounds had the higher increase in toxicity, i.e., were more toxic to SW-480 cells, in the presence of LB-100. FIG. 2C shows the cytotoxicity results in HT-29 colorectal cancer cells from the stress-focused drug screen of 164 drugs in the presence of LB-100 as compared to control. The indicated compounds had the higher increase in toxicity, i.e., were more toxic to HT-20 cells, in the presence of LB-100. FIG. 2D shows the cytotoxicity effects of adavosertib against colorectal cancer cell lines in a short-term cell viability assay. The cells were cultured with increasing concentrations of adavosertib for 5 days, then the cell viability was measured using resazurin. FIG. 2E shows the cytotoxicity effects of adavosertib against colorectal cancer cell lines in a longer-term cell proliferation assay. The cells were grown in the absence or presence of adavosertib at the indicated concentrations for 10-14 days, then fixed and stained. FIG. 2F shows the anti-proliferative effects of adavosertib, prexasertib, or A-1155463 alone or in combination with LB-100, in SW-480 colorectal cancer cells. Cells were grown in the presence of adavosertib, prexasertib or A-1155463 and in the absence or presence of LB-100, at the indicated concentrations for 10-14 days, then fixed and stained. FIG. 2G shows a comparison of cell viability curves for HT-29 and SW-480 cells treated with adavosertib or GDC-0575 alone (Control) or in the presence 2.5 µM LB-100. FIG. 2H shows a schematic outline of a genome-wide CRISPR screen of synthetic lethality. Cas9 expressing SW-480 cells were transduced with a lentiviral genome-wide gRNA library and three independent replicates were cultured with or without 2.5 µM LB-100 for 21 days. gRNA samples from T₀ and T₁₄ were recovered by PCR and quantified using next-generation sequencing. FIG. 2I show the results of a genome-wide CRISPR screen of FIG. 2H surveying for depleted genes that show synthetic lethality in cells treated with 2.5 µM LB-100. The graph provides a representation of the relative abundance of the gRNA sequences from the screen. The x-axis shows the log₂-transformed fold change (T_{treated}/T_{untreated}) and the y-axis shows the false discovery rate (FDR). FIG. 2J shows the results of a genome-wide CRISPR screen of FIG. 2H surveying for depleted genes that show synthetic lethality in cells treated with 6 µM LB-100. The graph provides a representation of the relative abundance of the gRNA sequences from the screen. The x-axis shows the log₂-transformed fold change (T_{treated}/T_{untreated}) and the y-axis shows the false discovery rate (FDR). FIG. 2K shows a CRISPRa screen carried out in an HT-29 cancer line to identify genes whose overexpression would increase LB-100 toxicity. This screen identified 53 genes whose overexpression is selectively toxic in the presence of LB-100. FIG. 2L shows that gRNAs targeting genes from the β-catenin (CTNNB1, BCL9L, and LEF1) or MAPK (MAPK14/p38α, MAPK1/ERK2) signaling pathways were significantly enriched in the samples treated with LB-100.

FIG. 3A shows visual representations of synergy matrices from various colorectal cancer cells treated with a combination of LB-100 and adavosertib. Cells were cultured with the indicated concentrations of LB-100 and adavosertib for 5 days, then the cell viability was measured using resazurin. The relative inhibition of cell viability is shown for each pair of concentrations. Synergy scores (ZIP) were calculated using the SynergyFinder 2.0 online tool (https://synergyfinder.org). Average synergy across the panel is highlighted at the box titled “Average”. FIG. 3B shows the anti-proliferative effects of sub-lethal concentrations of adavosertib (100 nM to 300 nM) alone or in combination with sub-lethal concentrations of LB-100 (2 µM or 4 µM), in various colorectal cancer cells. FIG. 3C shows the antiproliferative effects of adavosertib (100 nM, 200 nM, 300 nM, 400 nM, or 500 nM) alone or in combination with LB-100 (1 µM, 2 µM or 4 µM), in various colorectal cancer cells in a longer-term viability assay (10-14 days). FIG. 3D shows a measure of cell confluence over time compared to control in colorectal cancer cells treated with LB-100 (2 µM or 4 µM), adavosertib (200 nM or 400 nM), or a combination of LB-100 (2 µM or 4 µM) and adavosertib (200 nM or 400 nM). FIG. 3E shows synergy scores for a combination of LB-100 and adavosertib across 7 CRC lines. Cells were treated with LB-100 (1 µM, 2 µM, 3 µM, 4 µM, or 5 µM) and adavosertib (100 nM, 200 nM, 300 nM, 400 nM, or 500 nM) at all respective permutations for 4 days. The percentage of cell viability for each LB-100/adavosertib combination was estimated by resazurin fluorescence and normalized by DMSO controls. Synergyfinder web tool (https://synergyfinder.org) was used to calculate the ZIP synergy scores. Three independent experiments are represented.

FIG. 4A shows synergy matrices from colorectal cancer cells treated with a combination of LB-100 and prexasertib at indicated concentrations. FIG. 4B shows the antiproliferative effects of prexasertib alone or in combination with LB-100, in various colorectal cancer cell lines at indicated concentrations.

FIG. 5A shows the cytotoxicity results in RBE cholangiocarcinoma cells from the stress-focused drug screen of FIG. 2A of 164 drugs in the presence of LB-100 as compared to control. The indicated library compounds showed higher toxicity in the presence of LB-100. FIG. 5B shows cytotoxicity curves measuring cell confluence over time compared to control in RBE cholangiocarcinoma cells treated with LB-100, adavosertib, prexasertib, a combination of LB-100 and adavosertib or a combination of LB-100 and prexasertib. FIG. 5C shows the cytotoxicity effects of LB-100 and adavosertib against cholangiocarcinoma (CCA) cell lines in a short-term cell viability assay. The cells were cultured with increasing concentrations of LB-100 for 5 days, then the cell viability was measured using resazurin. FIG. 5D shows the cytotoxicity effects of LB-100 and adavosertib against cholangiocarcinoma cell lines in a longer-term cell proliferation assay, where cells were grown in the absence or presence of LB-100 at the indicated concentrations for 10-14 days, then fixed and stained. FIG. 5E shows cytotoxicity curves measuring cell confluence over time compared to control in various cholangiocarcinoma cell lines treated with LB-100, adavosertib, or a combination of LB-100 and adavosertib.

FIG. 6A shows the anti-proliferative effects of sub-lethal concentrations of adavosertib alone or in combination with sub-lethal concentrations of LB-100, and sub-lethal concentrations of prexasertib alone or in combination with sub-lethal concentrations of LB-100, in RBE cholangiocarcinoma cancer cell lines. FIG. 6B shows the anti-proliferative effects in a longer-term viability assay (10-14 days) of sub-lethal concentrations of adavosertib alone or in combination with sub-lethal concentrations of LB-100 (2 µm or 4 µm), in various cholangiocarcinoma cancer cell lines. FIG. 6C shows the anti-proliferative effects of sub-lethal concentrations of adavosertib alone or in combination with sub-lethal concentrations of LB-100 (2.5 µm or 5 µm), in various cholangiocarcinoma cancer cell lines. FIG. 6D shows the anti-proliferative effects of sub-lethal concentrations of prexasertib alone or in combination with sub-lethal concentrations of LB-100, in various cholangiocarcinoma cancer cell lines. FIG. 6E shows synergy scores for a combination of LB-100 and adavosertib across 4 cholangiocarcinoma (CCA) lines. Cells were treated with LB-100 (1 µM, 2 µM, 3 µM, 4 µM, or 5 µM) and adavosertib (100 nM, 200 nM, 300 nM, 400 nM, or 500 nM) at all respective permutations for 4 days. The percentage of cell viability for each condition was estimated by resazurin fluorescence and normalized by DMSO controls. Synergyfinder web tool (https://synergyfinder.org) was used to calculate the ZIP synergy scores. Three independent experiments are represented.

FIG. 7A shows the anti-proliferative effects of sub-lethal concentrations of adavosertib or prexasertib alone or in combination with sub-lethal concentrations of LB-100, in the high-grade ovarian cancer cell line OVCAR3. FIG. 7B shows the anti-proliferative effects of sub-lethal concentrations of adavosertib or prexasertib alone or in combination with sub-lethal concentrations of LB-100, in the high-grade ovarian cancer cell line SKOV3.

FIG. 8A shows the cytotoxicity effects of LB-100 against pancreatic ductal adenocarcinoma (PDAC) cell lines in a short-term cell viability assay. The cells were cultured with increasing concentrations of LB-100 for 5 days, then the cell viability was measured using resazurin. FIG. 8B shows the cytotoxicity effects of adavosertib against pancreatic ductal adenocarcinoma (PDAC) cell lines in a short-term cell viability assay. The cells were cultured with increasing concentrations of LB-100 for 5 days, then the cell viability was measured using resazurin. FIG. 8C shows the cytotoxicity effects of LB-100 or adavosertib against various PDAC cell lines in a longer-term cell proliferation assay (10-14 days). The cells were grown in the absence or presence of LB-100 (top panel) or adavosertib (bottom panel) at the indicated concentrations, then fixed and stained. FIG. 8D shows a measure of cell confluence over time compared to control in various PDAC cell lines treated with LB-100, adavosertib, or a combination of LB-100 and adavosertib. FIG. 8E shows the anti-proliferative effects in a longer-term viability assay (10-14 days) of sub-lethal concentrations of adavosertib alone or in combination with sub-lethal concentrations of LB-100 (1 µm, 2 µm or 4 µm), in various cholangiocarcinoma cancer cells. FIG. 8F shows synergy scores for a combination of LB-100 and adavosertib across 4 PDAC cell lines. Cells were treated with LB-100 (1 µM, 2 µM, 3 µM, 4 µM, or 5 µM) and adavosertib (100 nM, 200 nM, 300 nM, 400 nM, and 500 nM) at all respective permutations for 4 days. The percentage of cell viability for each condition was estimated by resazurin fluorescence and normalized by DMSO controls. Synergyfinder web tool (https://synergyfinder.org) was used to calculate the ZIP synergy scores. Three independent experiments are represented. FIG. 8G shows synergy scores for the indicated combinations across the 6 cancer cell lines. Cells were treated for 4 days with: adavosertib and LB-100; adavosertib and doxorubicin; adavosertib and gemcitabine; LB-100 and doxorubicin; or LB-100 and gemcitabine. The concentrations were: LB-100 (0.5 µM, 1 µM, 2 µM, 3 µM, 4 µM, 5 µM, 6 µM, 7 µM, 8 µM, or 9 µM); adavosertib (50 nM, 100 nM, 200 nM, 300 nM, 400 nM, 500 nM, 600 nM, 700 nM, 800 nM, or 900 nM); doxorubicin (5 nM, 10 nM, 20 nM, 30 nM, 40 nM, 50 nM, 60 nM, 70 nM, 80 nM, and 90 nM); Gemcitabine (0.63 nM, 1.25 nM, 2.5 nM, 5 nM, 10 nM, 20 nM, 30 nM, 40 nM, 50 nM, or 60 nM); and all respective permutations for the combinations were investigated. The percentage of cell viability for each combination was estimated by resazurin fluorescence and normalized by DMSO controls. Synergyfinder web tool (https://synergyfinder.org) was used to calculate the ZIP synergy scores. Three independent experiments are represented. FIG. 8H shows synergy matrices from various pancreatic cancer cell lines treated at indicated concentrations with a combination of LB-100 and adavosertib or a combination of LB-100 and prexasertib.

FIG. 9A shows the anti-proliferative effects in a longer-term viability assay of sub-lethal concentrations of adavosertib alone or in combination with sub-lethal concentrations of LB-100 (2 µm or 4 µm), in wild-type (ST) and combination-resistant (CR) CRC cell lines for over four months. FIG. 9B provides Western blots showing reduced oncogenic signaling in WT and CR colorectal cancer cell lines that have acquired resistance to treatment with LB-100 (2 µm or 4 µm) in combination with adavosertib. FIG. 9C is a graph comparing attached and Anchorage-independent proliferation in WT and CR SW-480 cells in the absence of drug (DMSO control) and with LB-100 and adavosertib combination treatment. FIG. 9D is a graph showing tumor volume reduction over a 50-day period in immunocompromised mice transplanted with SW-480 WT and SW-480 CR cancer cells.

### DETAILED DESCRIPTION OF THE INVENTION

### Definitions

The term “about” when immediately preceding a numerical value means ± 10% of the numerical value.

Throughout the present specification, numerical ranges are provided for certain quantities. These ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The compounds useful in the compositions and methods of the present invention can be the form of a pharmaceutically acceptable salt. Examples of pharmaceutically acceptable salts include, but are not limited to, mineral or organic acid salts of basic residues such as amines; alkali or organic salts of acidic residues such as phenols. Pharmaceutically acceptable salts can be obtained by reacting a compound functioning as a base, with an inorganic or organic acid to form a salt, for example, salts of hydrochloric acid, sulfuric acid, phosphoric acid, nitric acid, methanesulfonic acid, toluenesulfonic acid, acetic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. and bisulfate, valerate, oleate, palmitate, stearate, laurate, lactate, maleate, fumarate, tartrate, napthylate, glucoheptonate, lactobionate, and laurylsulphonate salts and the like. (see, e.g., Berge et al. (1977) “Pharmaceutical Salts”, J. Pharm. Sci. 66:1-19). Pharmaceutically acceptable salts can also be obtained by reacting a compound functioning as an acid with an inorganic or organic base to form a salt, for example, salts of sodium, potassium, lithium, ammonium, calcium, magnesium, iron, zinc, copper, manganese, aluminum, ammonia, isopropylamine, trimethylamine, etc. Those skilled in the art will further recognize that pharmaceutically acceptable salts can be prepared by reaction of the compound with an appropriate inorganic or organic acid or base via any of a number of known methods.

The compounds and pharmaceutically acceptable salts of the compounds useful in the methods and compositions of the invention are depicted showing relative stereochemistry. In some embodiments, the compounds and pharmaceutically acceptable salts of the compounds are enantiomers and are substantially free of their corresponding opposite enantiomer. The language “substantially free of” means includes no more than 5% of the minor enantiomer. In some embodiments, the compounds and pharmaceutically acceptable salts of the compounds are racemates. Unless otherwise indicated, the compounds and pharmaceutically acceptable salts of the compounds are racemates.

An “effective amount” when used in connection with LB-100 or an LB-100 ester, or a pharmaceutically acceptable salt thereof, means an amount of the compound that, when administered to a subject is effective to treat the cancer, or prevent, inhibit, or reduce risk of metastasis, alone or in combination with another anti-cancer agent.

An “effective amount” when used in connection with another anti-cancer agent, means an amount of the other anti-cancer agent that is effective to treat the cancer, or prevent, inhibit or reduce risk of metastasis, alone or in combination with LB-100 or an LB-100 ester, or a pharmaceutically acceptable salt thereof.

An “effective amount” when used in connection with (a) LB-100 or an LB-100 ester, or a pharmaceutically acceptable salt thereof, and (b) another anti-cancer agent, means a total amount of (a) and (b) that, when administered to a subject is effective to treat the cancer, or prevent, inhibit, or reduce risk of metastasis of a cancer.

An “effective amount” when used in connection with (a) LB-100 or an LB-100 ester, or a pharmaceutically acceptable salt thereof, (b) another anti-cancer agent and (c) another pharmaceutically active agent, means a total amount of (a), (b) and (c) that, when administered to a subject is effective to treat the cancer, or prevent, inhibit or reduce risk of metastasis of a cancer.

A “subject” is a human or non-human mammal, e.g., a bovine, horse, feline, canine, rodent, or non-human primate. The human can be a male or female, or child, adolescent or adult. The female can be premenarcheal or postmenarcheal.

All weight percentages (i.e., “% by weight” and “wt. %” and w/w) referenced herein, unless otherwise indicated, are relative to the total weight of the mixture or composition, as the case can be.

### LB-100 and Esters Thereof

### Lb-100

In some embodiments, the compositions of the invention comprise an effective amount of LB-100 and another anti-cancer agent. In some embodiments, the methods of the invention comprise administering an effective amount of LB-100 and another anti-cancer agent.

LB-100 has the structure

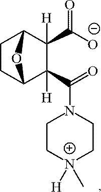

and exists as a racemic mixture of enantiomers having the structure of Enantiomer A and Enantiomer B:

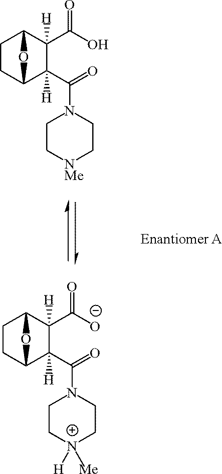 [SMILES: CN1CCN(C(=O)[C@@H]2[C@H](C(=O)O)[C@H]3CC[C@@H]2O3)CC1 ; C[NH+]1CCN(C(=O)[C@@H]2[C@H](C(=O)[O-])[C@H]3CC[C@@H]2O3)CC1]

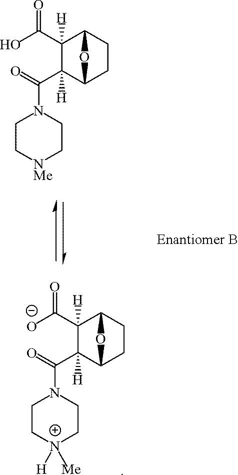

Each enantiomer can exist as a zwitterion.

In some embodiments, the compositions of the invention comprise an effective amount of an LB-100 ester or a pharmaceutically acceptable salt thereof and another anti-cancer agent. In some embodiments, the methods of the invention comprise administering an effective amount of an LB-100 ester or a pharmaceutically acceptable salt thereof and another anti-cancer agent. In some embodiments, the LB-100 ester is a compound having any one of the structures shown in Table A, or a pharmaceutically acceptable salt thereof.

TABLE A

| | |
|---|---|
| Compound No. | Structure |
| I-1 (LB-151) | 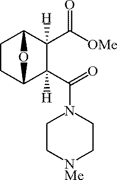 [SMILES: COC(=O)[C@H]1[C@@H](C(=O)N2CCN(C)CC2)[C@@H]2CC[C@H]1O2] |
| I-2 | 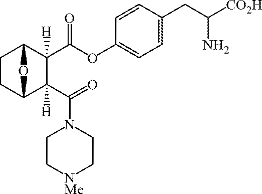 [SMILES: CN1CCN(C(=O)[C@@H]2[C@H](C(=O)Oc3ccc(CC(N)C(=O)O)cc3)[C@H]3CC[C@@H]2O3)CC1] |
| I-3 | 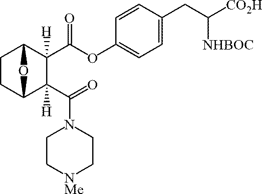 |
| I-4 (LB-113) | 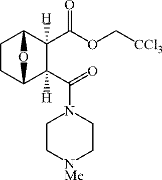 |
| I-5 | 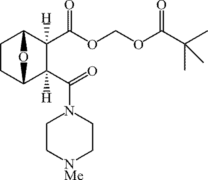 [SMILES: CN1CCN(C(=O)[C@@H]2[C@H](C(=O)OCOC(=O)C(C)(C)C)[C@H]3CC[C@@H]2O3)CC1] |
| I-6 | 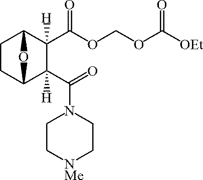 [SMILES: CCOC(=O)OCOC(=O)[C@H]1[C@@H](C(=O)N2CCN(C)CC2)[C@@H]2CC[C@H]1O2] |
| I-7 | 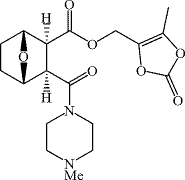 [SMILES: Cc1oc(=O)oc1COC(=O)[C@H]1[C@@H](C(=O)N2CCN(C)CC2)[C@@H]2CC[C@H]1O2] |
| I-8 | 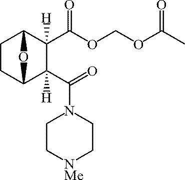 [SMILES: CC(=O)OCOC(=O)[C@H]1[C@@H](C(=O)N2CCN(C)CC2)[C@@H]2CC[C@H]1O2] |
| I-9 | 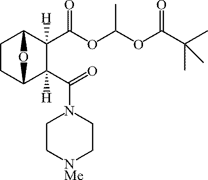 [SMILES: CC(OC(=O)[C@H]1[C@@H](C(=O)N2CCN(C)CC2)[C@@H]2CC[C@H]1O2)OC(=O)C(C)(C)C] |
| I-10 (LB-153) | 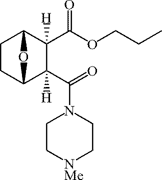 [SMILES: CCCOC(=O)[C@H]1[C@@H](C(=O)N2CCN(C)CC2)[C@@H]2CC[C@H]1O2] |
| I-11 (LB-157) | 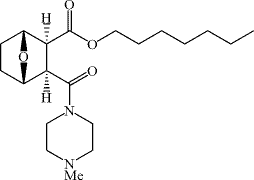 [SMILES: CCCCCCCOC(=O)[C@H]1[C@@H](C(=O)N2CCN(C)CC2)[C@@H]2CC[C@H]1O2] |

Methods for making LB-100 are disclosed in U.S. Pat. No. 7,998,957, which is incorporated herein by reference.

Methods for making LB-100 esters are disclosed in U.S. Pat. Nos. 11,236,102, 9,988,394, 9,994,584, 8,426,444, 7,998,957, and WO 2018/107004, each of which is incorporated herein by reference.

### Compositions of the Invention

The compositions of the invention comprise (1) an effective amount of (a) LB-100 or an LB-100 ester, or a pharmaceutically acceptable salt thereof and (b) another anti-cancer agent; and (2) a pharmaceutically acceptable carrier or vehicle. In some embodiments, the other anti-cancer agent is a WEE1 kinase inhibitor, CHK1 inhibitor, BCL-xL inhibitor, BCL-xL PROTAC, pan-BCL inhibitor, or ATR inhibitor. In some embodiments, the other anti-cancer agent is a WEE1 kinase inhibitor, CHK1 inhibitor, BCL-xL inhibitor, BCL-xL PROTAC, or pan-BCL inhibitor.

In some embodiments, the compositions of the invention comprise (1) an effective amount of (a) LB-100 or a pharmaceutically acceptable salt thereof and (b) another anti-cancer agent; and (2) a pharmaceutically acceptable carrier or vehicle. In some embodiments, the other anti-cancer agent is a WEE1 kinase inhibitor, CHK1 inhibitor, BCL-xL inhibitor, BCL-xL PROTAC, pan-BCL inhibitor, or ATR inhibitor. In some embodiments, the other anti-cancer agent is a WEE1 kinase inhibitor, CHK1 inhibitor, BCL-xL inhibitor, BCL-xL PROTAC, or pan-BCL inhibitor.

In some embodiments, the LB-100 ester is a compound having the structure depicted in Table A or a pharmaceutically acceptable salt thereof.

The compositions of the invention are useful for treating cancer, wherein the cancer is hepatocellular carcinoma (hepatoma), cholangiocarcinoma, colorectal carcinoma, small cell lung cancer, non-small cell lung cancer, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing’s tumor, leiomyosarcoma, rhabdomyosarcoma, pancreatic cancer, breast cancer, triple negative breast cancer, ovarian cancer, endometrial carcinoma, Fallopian tube cancer, prostate cancer, gastrointestinal stromal tumor (GIST), esophageal cancer, gallbladder cancer, gastrointestinal carcinoid tumor, duodenal cancer, gastroesophageal junction cancer, islet cell cancer, gastric cancer, anal cancer, cancer of the small intestine, pseudomyxoma peritonei, head and neck squamous cell carcinoma, Merkel cell carcinoma, tumor mutational burden-high cancer (TMB-H), microsatellite stable (MSS), mismatch repair proficient colon cancer, thyroid cancer, renal cell carcinoma, choriocarcinoma, seminoma, embryonal carcinoma, Wilms’ tumor, urothelial carcinoma, testicular cancer, bladder carcinoma, glioma, glioblastoma, multiforme, astrocytoma, medulloblastoma, craniopharyngioma, oligodendroglioma, malignant meningioma, diffuse intrinsic pontine glioma, melanoma, neuroblastoma, retinoblastoma, acute lymphoblastic B-cell leukemia, acute lymphoblastic T-cell leukemia, acute myeloblastic leukemia (AML), acute promyelocytic leukemia (APL), acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia (CML), chronic lymphocytic leukemia (CLL), hairy cell leukemia, multiple myeloma, lymphoblastic leukemia, myelogenous leukemia, lymphocytic leukemia, Hodgkin’s lymphoma, non-Hodgkin’s lymphoma, primary mediastinal large B-cell lymphoma, Waldenstrom’s macroglobulinemia, heavy chain disease, or polycythemia vera. In some embodiments, the cancer is colorectal cancer, cholangiocarcinoma, pancreatic cancer, or ovarian cancer. In some embodiments, the non-small cell lung cancer is lung adenocarcinoma, lung squamous carcinoma or lung large cell carcinoma. In some embodiments, the thyroid cancer is anaplastic thyroid cancer or follicular thyroid cancer. In some embodiments, the cancer is HER2-positive. In some embodiments, the cancer is HER2-negative. In some embodiments, the cancer expresses a BRCA1 or BRCA2 gene oncogenic mutation. In some embodiments, the cancer does not express a BRCA1 or BRCA2 gene oncogenic mutation.

The compositions of the invention are also useful for preventing, inhibiting, or reducing risk of metastasis of a cancer, wherein the cancer is hepatocellular carcinoma (hepatoma), cholangiocarcinoma, colorectal carcinoma, small cell lung cancer, non-small cell lung cancer, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing’s tumor, leiomyosarcoma, rhabdomyosarcoma, pancreatic cancer, breast cancer, triple negative breast cancer, ovarian cancer, endometrial carcinoma, Fallopian tube cancer, prostate cancer, gastrointestinal stromal tumor (GIST), esophageal cancer, gallbladder cancer, gastrointestinal carcinoid tumor, duodenal cancer, gastroesophageal junction cancer, islet cell cancer, gastric cancer, anal cancer, cancer of the small intestine, pseudomyxoma peritonei, head and neck squamous cell carcinoma, Merkel cell carcinoma, tumor mutational burden-high cancer (TMB-H), microsatellite stable (MSS), mismatch repair proficient colon cancer, thyroid cancer, renal cell carcinoma, choriocarcinoma, seminoma, embryonal carcinoma, Wilms’ tumor, urothelial carcinoma, testicular cancer, bladder carcinoma, glioma, glioblastoma, multiforme, astrocytoma, medulloblastoma, craniopharyngioma, oligodendroglioma, malignant meningioma, diffuse intrinsic pontine glioma, melanoma, neuroblastoma, retinoblastoma, acute lymphoblastic B-cell leukemia, acute lymphoblastic T-cell leukemia, acute myeloblastic leukemia (AML), acute promyelocytic leukemia (APL), acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia (CML), chronic lymphocytic leukemia (CLL), hairy cell leukemia, multiple myeloma, lymphoblastic leukemia, myelogenous leukemia, lymphocytic leukemia, Hodgkin’s lymphoma, non-Hodgkin’s lymphoma, primary mediastinal large B-cell lymphoma, Waldenstrom’s macroglobulinemia, heavy chain disease, or polycythemia vera. In some embodiments, the cancer is colorectal cancer, cholangiocarcinoma, pancreatic cancer or ovarian cancer. In some embodiments, the non-small cell lung cancer is lung adenocarcinoma, lung squamous carcinoma or lung large cell carcinoma. In some embodiments, the thyroid cancer is anaplastic thyroid cancer or follicular thyroid cancer. In some embodiments, the cancer is HER2-positive. In some embodiments, the cancer is HER2-negative. In some embodiments, the cancer expresses a BRCA1 or BRCA2 gene oncogenic mutation. In some embodiments, the cancer does not express a BRCA1 or BRCA2 gene oncogenic mutation.

In some embodiments, the pharmaceutically acceptable carrier or vehicle comprises monosodium glutamate or has a pH of 10-11. In some embodiments, the pharmaceutically acceptable carrier or vehicle comprises monosodium glutamate and has a pH of 10-11. Illustrative compositions comprising monosodium glutamate and having a pH of 10-11 are disclosed in U.S. Pat. No. 10,532,050, which is incorporated herein by reference in its entirety.

In some embodiments, LB-100 or an LB-100 ester, or a pharmaceutically acceptable salt thereof is present in the compositions of the invention at a concentration of about 1.0 mg/mL and/or the monosodium glutamate is present in the compositions of the invention at a concentration of about 0.1 M.

In some embodiments, the pH of the compositions of the invention is about 10.4 to about 10.6. In some embodiments, the pH of the compositions of the invention is about 10.5.

In some embodiments, the compositions of the invention further comprise water.

In some embodiments, the compositions of the invention comprise about 0.15 mg to about 20 mg of an LB-100 ester or pharmaceutically acceptable salt thereof, e.g., about 0.15 mg, about 0.25 mg, about 0.5 mg, about 0.75 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, or about 20 mg, including all values and subranges therebetween. In some embodiments, the compositions of the invention comprise about 1 mg to about 20 mg of an LB-100 ester or pharmaceutically acceptable salt thereof. In some embodiments, the compositions of the invention comprise about 5 mg to about 20 mg of and LB-100 ester or pharmaceutically acceptable salt thereof. In some embodiments, the compositions of the invention comprise about 10 mg to about 20 mg of and LB-100 ester or pharmaceutically acceptable salt thereof. In some embodiments, the compositions of the invention comprise about 15 mg to about 20 mg of and LB-100 ester or pharmaceutically acceptable salt thereof. In some embodiments, the compositions of the invention comprise about 1 mg to about 15 mg of an LB-100 ester or pharmaceutically acceptable salt thereof. In some embodiments, the compositions of the invention comprise about 1 mg to about 10 mg of an LB-100 ester or pharmaceutically acceptable salt thereof. In some embodiments, the compositions of the invention comprise about 1 mg to about 5 mg of an LB-100 ester or pharmaceutically acceptable salt thereof. In some embodiments, the compositions of the invention comprise about 5 mg to about 10 mg of an LB-100 ester or pharmaceutically acceptable salt thereof. In some embodiments, the compositions of the invention comprise about 0.1 mg/m² to about 10 mg/m² of an LB-100 ester or pharmaceutically acceptable salt thereof.

### Other Anti-Cancer Agents

In some embodiments, the compositions of the invention comprise (1) an effective amount of (a) LB-100 or an LB-100 ester, or a pharmaceutically acceptable salt thereof; and (b) a WEE1 kinase inhibitor; and (2) a pharmaceutically acceptable carrier or vehicle.

In some embodiments, the WEE1 kinase inhibitor is adavosertib, PD0166285, PD407824, ZN-c3, IMP7068, Debio 0123, SDGR2, SY4835, or NUV-569. In some embodiments, the WEE1 kinase inhibitor is PD0166285. In some embodiments, the WEE1 kinase inhibitor is adavosertib.

In some embodiments, the compositions of the invention comprise (1) an effective amount of (a) LB-100 and (b) adavosertib; and (2) a pharmaceutically acceptable carrier or vehicle.

In some embodiments, the compositions of the invention comprise about 25 mg to about 500 mg of the WEE1 kinase inhibitor, e.g., about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg, including all values and subranges therebetween. In some embodiments, the compositions of the invention comprise about 50 mg to about 400 mg of the WEE1 kinase inhibitor. In some embodiments, the compositions of the invention comprise about 50 mg of the WEE1 kinase inhibitor. In some embodiments, the compositions of the invention comprise about 100 mg of the WEE1 kinase inhibitor. In some embodiments, the compositions of the invention comprise about 125 mg of the WEE1 kinase inhibitor. In some embodiments, the compositions of the invention comprise about 150 mg of the WEE1 kinase inhibitor. In some embodiments, the compositions of the invention comprise about 175 mg of the WEE1 kinase inhibitor. In some embodiments, the compositions of the invention comprise about 200 mg of the WEE1 kinase inhibitor. In some embodiments, the compositions of the invention comprise about 225 mg of the WEE1 kinase inhibitor. In some embodiments, the compositions of the invention comprise about 250 mg of the WEE1 kinase inhibitor. In some embodiments, the compositions of the invention comprise about 300 mg of the WEE1 kinase inhibitor. In some embodiments, the compositions of the invention comprise about 400 mg of the WEE1 kinase inhibitor.

In some embodiments, the WEE1 kinase inhibitor is adavosertib. In some embodiments, the WEE1 kinase inhibitor is adavosertib and the compositions of the invention comprise about 100 mg to about 400 mg of adavosertib, e.g., about 100 mg, about 125 mg, about 150 mg, about 175 mg, about 200 mg, about 225 mg, about 250 mg, about 275 mg, about 300 mg, about 325 mg, about 350 mg, about 375 mg, or about 400 mg, including all values and subranges therebetween. In some embodiments, the WEE1 kinase inhibitor is adavosertib and the compositions of the invention comprise about 125 mg or about 300 mg of adavosertib. In some embodiments, the WEE1 kinase inhibitor is adavosertib and the compositions of the invention comprise about 125 mg, 175 mg, 200 mg, 225 mg, 300 mg, or about 400 mg of adavosertib.

In some embodiments, the compositions of the invention comprise (1) an effective amount of (a) LB-100 or an LB-100 ester, or a pharmaceutically acceptable salt thereof; and (b) a CHK1 inhibitor; and (2) a pharmaceutically acceptable carrier or vehicle.

In some embodiments, the CHK1 inhibitor is GDC-0575, prexasertib, rabusertib, SCH-900776, CCT-245737, AZD-7762, PF-477736, GDC-0425, or SRA737.

In some embodiments, the compositions of the invention comprise (1) an effective amount of (a) LB-100 and (b) prexasertib; and (2) a pharmaceutically acceptable carrier or vehicle.

In some embodiments, the compositions of the invention comprise about 5 mg to about 100 mg of the CHK1 inhibitor, e.g., about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg, including all values and subranges therebetween. In some embodiments, the compositions of the invention comprise about 10 mg to about 50 mg of the CHK1 inhibitor. In some embodiments, the compositions of the invention comprise about 5 mg of the CHK1 inhibitor. In some embodiments, the compositions of the invention comprise about 10 mg of the CHK1 inhibitor. In some embodiments, the compositions of the invention comprise about 15 mg of the CHK1 inhibitor. In some embodiments, the compositions of the invention comprise about 20 mg of the CHK1 inhibitor. In some embodiments, the compositions of the invention comprise about 25 mg of the CHK1 inhibitor. In some embodiments, the compositions of the invention comprise about 30 mg of the CHK1 inhibitor. In some embodiments, the compositions of the invention comprise about 35 mg of the CHK1 inhibitor. In some embodiments, the compositions of the invention comprise about 40 mg of the CHK1 inhibitor. In some embodiments, the compositions of the invention comprise about 45 mg of the CHK1 inhibitor. In some embodiments, the compositions of the invention comprise about 50 mg of the CHK1 inhibitor.

In some embodiments, the CHK1 inhibitor is rabusertib. In some embodiments, the CHK1 inhibitor is rabusertib and the compositions of the invention comprise about 150 mg to about 500 mg of rabusertib, e.g., about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg, including all values and subranges therebetween. In some embodiments, the CHK1 inhibitor is rabusertib and the compositions of the invention comprise about 150 mg or about 250 mg of rabusertib. In some embodiments, the CHK1 inhibitor is rabusertib and the compositions of the invention comprise about 170 mg or about 230 mg of rabusertib.

In some embodiments, the CHK1 inhibitor is prexasertib. In some embodiments, the CHK1 inhibitor is prexasertib and the compositions of the invention comprise about 150 mg to about 500 mg of prexasertib, e.g., about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg, including all values and subranges therebetween. In some embodiments, the CHK1 inhibitor is prexasertib and the compositions of the invention comprise about 150 mg or about 250 mg of prexasertib. In some embodiments, the CHK1 inhibitor is prexasertib and the compositions of the invention comprise about 170 mg or about 230 mg of prexasertib.

In some embodiments, the compositions of the invention comprise (1) an effective amount of (a) LB-100 or an LB-100 ester, or a pharmaceutically acceptable salt thereof; and (b) a BCL-xL inhibitor; and (2) a pharmaceutically acceptable carrier or vehicle.

In some embodiments, the BCL-xL inhibitor is A-1155463.

In some embodiments, the compositions of the invention comprise (1) an effective amount of (a) LB-100 and (b) A-1155463; and (2) a pharmaceutically acceptable carrier or vehicle.

In some embodiments, the compositions of the invention comprise 100 mg to about 400 mg of A-1 155463, e.g., about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, including all values and subranges therebetween.

In some embodiments, the compositions of the invention comprise (1) an effective amount of (a) LB-100 or an LB-100 ester, or a pharmaceutically acceptable salt thereof; and (b) BCL-xL PROTAC; and (2) a pharmaceutically acceptable carrier or vehicle.

In some embodiments, the BCL-xL PROTAC is DT2216 or PZ15227.

In some embodiments, the compositions of the invention comprise (1) an effective amount of (a) LB-100 and (b) DT2216 or PZ15227; and (2) a pharmaceutically acceptable carrier or vehicle.

In some embodiments, the compositions of the invention comprise 1 mg to about 400 mg of DT2216 or PZ15227, e.g., about 1 mg, about 5 mg, about 10 mg, about 20 mg, about 30 mg, about 40 mg, about 50 mg, about 60 mg, about 70 mg, about 80 mg, about 90 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, including all values and subranges therebetween. In some embodiments, the compositions of the invention comprise about 50 mg to about 400 mg of DT2216 or PZ15227. In some embodiments, the compositions of the invention comprise about 50 mg to about 200 mg of DT2216 or PZ15227.

In some embodiments, the compositions of the invention comprise (1) an effective amount of (a) LB-100 or an LB-100 ester, or a pharmaceutically acceptable salt thereof, and (b) a pan-BCL inhibitor; and (2) a pharmaceutically acceptable carrier or vehicle.

In some embodiments, the pan-BCL inhibitor is navitoclax.

In some embodiments, the compositions of the invention comprise (1) an effective amount of (a) LB-100 and (b) navitoclax; and (2) a pharmaceutically acceptable carrier or vehicle.

In some embodiments, the compositions of the invention comprise about 100 mg to about 400 mg of navitoclax, e.g., about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, including all values and subranges therebetween. In some embodiments, the compositions of the invention comprise about 150 mg to about 325 mg of navitoclax. In some embodiments, the compositions of the invention comprise about 150 mg or about 325 mg of navitoclax.

In some embodiments, the compositions of the invention comprise (1) an effective amount of (a) LB-100 or an LB-100 ester, or a pharmaceutically acceptable salt thereof; and (b) an ATR inhibitor; and (2) a pharmaceutically acceptable carrier or vehicle.

In some embodiments, the ATR inhibitor is berzosertib (M6620), camonsertib (RP-3500), ceralasertib (AZD6738), elimusertib (BAY1895344), dactolisib, SKLB-197, AZ20, VE-821, VX-803, ETP-46464, ATG-018, schisandrin B, CGK 733, torin 2, or HAMNO. In some embodiments, the ATR inhibitor is berzosertib.

Tables D1-D3 set forth illustrative Compositions A1-A12, B1-B12, C1-C12, D1-D12, E1-E12, F1-F12, G1-G12, H1-H12, I1-I12, J1-J12, K1-K12, L1-L12, M1-M12, N1-N12, O1-O12, P1-P12, Q1-Q12, R1-R12, S1-S12, T1-T12, U1-U12, V1-V12 and W1-W12. Each Composition of Tables D1-D3 comprises (1) an effective amount of (a) LB-100 or an LB-100 ester, or a pharmaceutically acceptable salt thereof, and (b) an anti-cancer agent; and (2) a pharmaceutically acceptable carrier or vehicle. For example, Composition A1 comprises (1) an effective amount of (a) Compound I-1(or a pharmaceutically acceptable salt thereof) and (b) adavosertib; and (2) a pharmaceutically acceptable carrier or vehicle; Composition A2 comprises (1) an effective amount of (a) Compound I-2 (or a pharmaceutically acceptable salt thereof) and (b) adavosertib; and (2) a pharmaceutically acceptable carrier or vehicle; etc.

TABLE D1

Compositions

| | | 1 | 2 | 3 | 4 |
|---|---|---|---|---|---|
| | | Compound I-1 or a pharmaceutical ly acceptable salt thereof | Compound I-2 or a pharmaceutical ly acceptable salt thereof | Compound I-3 or a pharmaceutical ly acceptable salt thereof | Compound I-4 or a pharmaceutical ly acceptable salt thereof |
| A | adavosertib | A1 | A2 | A3 | A4 |
| B | PD0166285 | B1 | B2 | B3 | B4 |
| C | PD407824 | C1 | C2 | C3 | C4 |
| D | ZN-c3 | D1 | D2 | D3 | D4 |
| E | IMP7068 | E1 | E2 | E3 | E4 |
| F | Debio 0123 | F1 | F2 | F3 | F4 |
| G | SDGR2 | G1 | G2 | G3 | G4 |
| H | NUV-569 | H1 | H2 | H3 | H4 |
| I | rabusertib | I1 | I2 | I3 | I4 |
| J | prexasertib | J1 | J2 | J3 | J4 |
| K | GDC-0575 | K1 | K2 | K3 | K4 |
| L | SCH-900776 | L1 | L2 | L3 | L4 |
| M | AZD-7762 | M1 | M2 | M3 | M4 |
| N | PF-477736 | N1 | N2 | N3 | N4 |
| O | GDC-0425 | O1 | O2 | O3 | O4 |
| P | SRA737 | P1 | P2 | P3 | P4 |
| Q | A-1155463 | Q1 | Q2 | Q3 | Q4 |
| R | navitoclax | R1 | R2 | R3 | R4 |
| S | CCT-245737 | S1 | S2 | S3 | S4 |
| T | SY4835 | T1 | T2 | T3 | T4 |
| U | berzosertib | U1 | U2 | U3 | U4 |
| V | elimusertib | V1 | V2 | V3 | V4 |
| W | camonsertib | W1 | W2 | W3 | W4 |

TABLE D2

Compositions

| | | 5 | 6 | 7 | 8 |
|---|---|---|---|---|---|
| | | Compound I-5 or a pharmaceutical ly acceptable salt thereof | Compound I-6 or a pharmaceutical ly acceptable salt thereof | Compound I-7 or a pharmaceutical ly acceptable salt thereof | Compound I-8 or a pharmaceutical ly acceptable salt thereof |
| A | adavosertib | A5 | A6 | A7 | A8 |
| B | PD0166285 | B5 | B6 | B7 | B8 |
| C | PD407824 | C5 | C6 | C7 | C8 |
| D | ZN-c3 | D5 | D6 | D7 | D8 |
| E | IMP7068 | E5 | E6 | E7 | E8 |
| F | Debio 0123 | F5 | F6 | F7 | F8 |
| G | SDGR2 | G5 | G6 | G7 | G8 |
| H | NUV-569 | H5 | H6 | H7 | H8 |
| I | rabusertib | I5 | I6 | I7 | I8 |
| J | prexasertib | J5 | J6 | J7 | J8 |
| K | GDC-0575 | K5 | K6 | K7 | K8 |
| L | SCH-900776 | L5 | L6 | L7 | L8 |
| M | AZD-7762 | M5 | M6 | M7 | M8 |
| N | PF-477736 | N5 | N6 | N7 | N8 |
| O | GDC-0425 | O5 | O6 | O7 | O8 |
| P | SRA737 | P5 | P6 | P7 | P8 |
| Q | A-1155463 | Q5 | Q6 | Q7 | Q8 |
| R | navitoclax | R5 | R6 | R7 | R8 |
| S | CCT-245737 | S5 | S6 | S7 | S8 |
| T | SY4835 | T5 | T6 | T7 | T8 |
| U | berzosertib | U5 | U6 | U7 | U8 |
| V | elimusertib | V5 | V6 | V7 | V8 |
| W | camonsertib | W5 | W6 | W7 | W8 |

TABLE D3

Compositions

| | | 9 | 10 | 11 | 12 |
|---|---|---|---|---|---|
| | | Compound I-9 or a pharmaceutical ly acceptable salt thereof | Compound I-10 or a pharmaceutical ly acceptable salt thereof | Compound I-11 or a pharmaceutical ly acceptable salt thereof | LB-100 or a pharmaceutical ly acceptable salt thereof |
| A | adavosertib | A9 | A10 | A11 | A12 |
| B | PD0166285 | B9 | B10 | B11 | B12 |
| C | PD407824 | C9 | C10 | C11 | C12 |
| D | ZN-c3 | D9 | D10 | D11 | D12 |
| E | IMP7068 | E9 | E10 | E11 | E12 |
| F | Debio 0123 | F9 | F10 | F11 | F12 |
| G | SDGR2 | G9 | G10 | G11 | G12 |
| H | NUV-569 | H9 | H10 | H11 | H12 |
| I | rabusertib | I9 | I10 | I11 | I12 |
| J | prexasertib | J9 | J10 | J11 | J12 |
| K | GDC-0575 | K9 | K10 | K11 | K12 |
| L | SCH-900776 | L9 | L10 | L11 | L12 |
| M | AZD-7762 | M9 | M10 | M11 | M12 |
| N | PF-477736 | N9 | N10 | N11 | N12 |
| O | GDC-0425 | O9 | O10 | O11 | O12 |
| P | SRA737 | P9 | P10 | P11 | P12 |
| Q | A-1155463 | Q9 | Q10 | Q11 | Q12 |
| R | navitoclax | R9 | R10 | R11 | R12 |
| S | CCT-245737 | S9 | S10 | S11 | S12 |
| T | SY4835 | T9 | T10 | T11 | T12 |
| U | berzosertib | U9 | U10 | U11 | U12 |
| V | elimusertib | V9 | V10 | V11 | V12 |
| W | camonsertib | W9 | W10 | W11 | W12 |

In some embodiments, the compositions of the invention comprise (a) LB-100 or an LB-100 ester, or a pharmaceutically acceptable salt thereof and (b) another anti-cancer agent, in an amount of (a) or (b) that is about 10 wt% to about 99 wt% of the total weight of the composition of the invention. In some embodiments, the other anti-cancer agent is a WEE1 kinase inhibitor, CHK1 inhibitor, BCL-xL inhibitor, BCL-xL PROTAC, pan-BCL inhibitor or ATR inhibitor. In some embodiments, the anti-cancer agent is a WEE1 kinase inhibitor, CHK1 inhibitor, BCL-xL inhibitor, BCL-xL PROTAC or pan-BCL inhibitor.

In some embodiments, the compositions of the invention comprise (a) LB-100 or an LB-100 ester, or a pharmaceutically acceptable salt thereof, and (b) another anti-cancer agent, in an amount of (a) and (b) that is about 10 wt% to about 99 wt% of the total weight of the composition of the invention. In some embodiments, the other anti-cancer agent is a WEE1 kinase inhibitor, CHK1 inhibitor, BCL-xL inhibitor, BCL-xL PROTAC, pan-BCL inhibitor or ATR inhibitor. In some embodiments, the anti-cancer agent is a WEE1 kinase inhibitor, CHK1 inhibitor, BCL-xL inhibitor, BCL-xL PROTAC or pan-BCL inhibitor.

In some embodiments, the compositions of the invention comprise (a) LB-100.

In some embodiments, the compositions of the invention further comprise an effective amount of another pharmaceutically active agent.

In some embodiments, the other pharmaceutically active agent is an immunotherapeutic agent. In some embodiments, the immunotherapeutic agent is dostarlimab-gxly, ticilimumab, avelumab, pembrolizumab, avelumab, durvalumab, nivolumab, cemiplimab, ABX196, sintilimab, camrelizumab, spartalizumab, toripalimab, bispecific antibody XmAb20717, mapatumumab, tremelimumab, carotuximab, tocilizumab, ipilimumab, atezolizumab, bevacizumab, ramucirumab, IBI305, ascrinvacumab, TCR T-cell therapy agent, cytokine-based biologic agent IRX-2, bempegaldesleukin, DKN-01, PTX-9908, AK104, PT-112, SRF388, ET1402L1-CART, Glypican 3-specific Chimeric Antigen Receptor Expressing T Cells (CAR-T cells), CD147-targeted CAR-T cells, NKG2D-based CAR T-cells, or neoantigen reactive T cells.

In some embodiments, the other pharmaceutically active agent is a chemotherapeutic agent. In some embodiments, the chemotherapeutic agent is altretamine, dendmustine, busulfan, carboplatin, chlorambucil, cisplatic, cyclophosphamide, dacarbazine, ifosamide, lomustine, mechlorethamine, melphalan, oxaliplatin, temozolomide, thiotepa, trabectedin, streptozocin, azacytidine, 5-fluorouracil (5-FU), 6-mercaptopurine (6-MP), capecitabine, cladribine, clofarabine, cytarabine, decitabine, floxuridine, fludarabine, gemcitabine, hydroxyurea, methotrexate, nelarabine, pemetrexed, pentostatin, pralatrexate, thioguanine, trifluridine/tipiracil, daunorubicin, doxorubicin, epirubicin, idarubicin, valrubicin, bleomycin, dactinomycin, mitomycin C, mitoxantrone, irinotecan, topotecan, etoposide, teniposide, cabizitaxel, docetaxel, nab-paclitaxel, paclitaxel, vinblastine, vincristine, vinorelbine, eribulin, ixabepilone, mitotane, omacetaxine, procarbazine, romidepsin, vorinostat, prednisone, methylprednisone, dexamethasone, tamoxifen, sitravatinib or leuprolide. In some embodiments, the compositions of the invention are suitable for injection or intravenous infusion. In some embodiments, the pharmaceutically acceptable carrier or vehicle comprises monosodium glutamate or has a pH of about 10 to about11. In some embodiments, the pharmaceutically acceptable carrier or vehicle comprises monosodium glutamate and has a pH of about 10 to about 11. In some embodiments, the pharmaceutically acceptable carrier or vehicle comprises monosodium glutamate or has a pH of 10-11. In some embodiments, the pharmaceutically acceptable carrier or vehicle comprises monosodium glutamate and has a pH of 10-11.

In some embodiments, LB-100 or an LB-100 ester, or pharmaceutically acceptable salt thereof is present in the pharmaceutical composition at a concentration of about 1.0 mg/mL. In some embodiments, LB-100 or an LB-100 ester, or pharmaceutically acceptable salt thereof is present in the pharmaceutical composition at a concentration of 1.0 mg/mL.

In some embodiments, the monosodium glutamate is present in the pharmaceutical composition at a concentration of about 0.1 M. In some embodiments, the monosodium glutamate is present in the pharmaceutical composition at a concentration of 0.1 M.

In some embodiments, the pH of the pharmaceutical composition is about 10.4 to about 10.6. In some embodiments, the pH of the pharmaceutical composition is about 10.5. In some embodiments, the pH of the pharmaceutical composition is 10.4-10.6. In some embodiments, the pH of the pharmaceutical composition is 10.5.

The following delivery systems are only representative of the many possible systems useful for administering compositions in accordance with the invention.

Suitable routes of administration by injection include parenteral administration, such as intramuscular, intravenous, or subcutaneous administration. Administration of a composition of the invention by infusion can be carried out in a variety of conventional ways, such as cutaneous, subcutaneous, intraperitoneal, parenteral, intraarterial or intravenous injection. In some embodiments, the composition of the invention is administered intravenously. In some embodiments, the composition of the invention is administered subcutaneously.

Alternately, one may administer the composition of the invention in a local rather than systemic manner, for example, via injection of the composition directly into the site of action, often in a depot or sustained release formulation. Furthermore, one may administer the composition of the invention in a targeted drug delivery system.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprolactones and PLGA’s).

Other injectable drug delivery systems include solutions, suspensions, gels. Oral delivery systems include tablets and capsules. These can contain excipients such as binders (e.g., hydroxypropylmethylcellulose, polyvinylpyrrolidone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Implantable systems include rods and discs, and can contain excipients such as PLGA and polycaprolactone.

Oral delivery systems include tablets and capsules. These can contain excipients such as binders (e.g., hydroxypropylmethylcellulose, polyvinylpyrrolidone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

As used herein, “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

### Methods of the Invention

The present invention provides methods for treating cancer, comprising administering to a subject in need thereof an effective amount of: (a) LB-100 or an LB-100 ester, or a pharmaceutically acceptable salt thereof and (b) a WEE1 kinase inhibitor, CHK1 inhibitor, BCL-xL inhibitor, BCL-xL PROTAC, pan-BCL inhibitor or ATR inhibitor, wherein the cancer is hepatocellular carcinoma (hepatoma), cholangiocarcinoma, colorectal carcinoma, small cell lung cancer, non-small cell lung cancer, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing’s tumor, leiomyosarcoma, rhabdomyosarcoma, pancreatic cancer, breast cancer, triple negative breast cancer, ovarian cancer, endometrial carcinoma, Fallopian tube cancer, prostate cancer, gastrointestinal stromal tumor (GIST), esophageal cancer, gallbladder cancer, gastrointestinal carcinoid tumor, duodenal cancer, gastroesophageal junction cancer, islet cell cancer, gastric cancer, anal cancer, cancer of the small intestine, pseudomyxoma peritonei, head and neck squamous cell carcinoma, Merkel cell carcinoma, tumor mutational burden-high cancer (TMB-H), microsatellite stable (MSS), mismatch repair proficient colon cancer, thyroid cancer, renal cell carcinoma, choriocarcinoma, seminoma, embryonal carcinoma, Wilms’ tumor, urothelial carcinoma, testicular cancer, bladder carcinoma, glioma, glioblastoma, multiforme, astrocytoma, medulloblastoma, craniopharyngioma, oligodendroglioma, malignant meningioma, diffuse intrinsic pontine glioma, melanoma, neuroblastoma, retinoblastoma, acute lymphoblastic B-cell leukemia, acute lymphoblastic T-cell leukemia, acute myeloblastic leukemia (AML), acute promyelocytic leukemia (APL), acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia (CML), chronic lymphocytic leukemia (CLL), hairy cell leukemia, multiple myeloma, lymphoblastic leukemia, myelogenous leukemia, lymphocytic leukemia, Hodgkin’s lymphoma, non-Hodgkin’s lymphoma, primary mediastinal large B-cell lymphoma, Waldenstrom’s macroglobulinemia, heavy chain disease, or polycythemia vera. In some embodiments, the cancer is colorectal cancer, cholangiocarcinoma, pancreatic cancer or ovarian cancer. In some embodiments, the non-small cell lung cancer is lung adenocarcinoma, lung squamous carcinoma, or lung large cell carcinoma. In some embodiments, the thyroid cancer is anaplastic thyroid cancer or follicular thyroid cancer. In some embodiments, the cancer is human epidermal growth factor receptor 2 (HER2)-positive. In some embodiments, the cancer is HER2-negative. In some embodiments, the cancer expresses a BRCA1 or BRCA2 gene oncogenic mutation. In some embodiments, the cancer does not express a BRCA1 or BRCA2 gene oncogenic mutation.

The present invention also provides methods for treating cancer, comprising administering to a subject in need thereof an effective amount of: (a) LB-100 or an LB-100 ester, or a pharmaceutically acceptable salt thereof and (b) a WEE1 kinase inhibitor, CHK1 inhibitor, BCL-xL inhibitor, BCL-xL PROTAC or pan-BCL inhibitor, wherein the cancer is hepatocellular carcinoma (hepatoma), cholangiocarcinoma, colorectal carcinoma, small cell lung cancer, non-small cell lung cancer, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing’s tumor, leiomyosarcoma, rhabdomyosarcoma, pancreatic cancer, breast cancer, triple negative breast cancer, ovarian cancer, endometrial carcinoma, Fallopian tube cancer, prostate cancer, gastrointestinal stromal tumor (GIST), esophageal cancer, gallbladder cancer, gastrointestinal carcinoid tumor, duodenal cancer, gastroesophageal junction cancer, islet cell cancer, gastric cancer, anal cancer, cancer of the small intestine, pseudomyxoma peritonei, head and neck squamous cell carcinoma, Merkel cell carcinoma, tumor mutational burden-high cancer (TMB-H), microsatellite stable (MSS), mismatch repair proficient colon cancer, thyroid cancer, renal cell carcinoma, choriocarcinoma, seminoma, embryonal carcinoma, Wilms’ tumor, urothelial carcinoma, testicular cancer, bladder carcinoma, glioma, glioblastoma, multiforme, astrocytoma, medulloblastoma, craniopharyngioma, oligodendroglioma, malignant meningioma, diffuse intrinsic pontine glioma, melanoma, neuroblastoma, retinoblastoma, acute lymphoblastic B-cell leukemia, acute lymphoblastic T-cell leukemia, acute myeloblastic leukemia (AML), acute promyelocytic leukemia (APL), acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia (CML), chronic lymphocytic leukemia (CLL), hairy cell leukemia, multiple myeloma, lymphoblastic leukemia, myelogenous leukemia, lymphocytic leukemia, Hodgkin’s lymphoma, non-Hodgkin’s lymphoma, primary mediastinal large B-cell lymphoma, Waldenstrom’s macroglobulinemia, heavy chain disease, or polycythemia vera. In some embodiments, the cancer is colorectal cancer, cholangiocarcinoma, pancreatic cancer or ovarian cancer. In some embodiments, the non-small cell lung cancer is lung adenocarcinoma, lung squamous carcinoma, or lung large cell carcinoma. In some embodiments, the thyroid cancer is anaplastic thyroid cancer or follicular thyroid cancer. In some embodiments, the cancer is human epidermal growth factor receptor 2 (HER2)-positive. In some embodiments, the cancer is HER2-negative. In some embodiments, the cancer expresses a BRCA1 or BRCA2 gene oncogenic mutation. In some embodiments, the cancer does not express a BRCA1 or BRCA2 gene oncogenic mutation.

The present invention also provides methods for preventing, inhibiting, or reducing risk of metastasis of a cancer, comprising administering to a subject in need thereof an effective amount of: (a) LB-100 or an LB-100 ester, or a pharmaceutically acceptable salt thereof and (b) a WEE1 kinase inhibitor, CHK1 inhibitor, BCL-xL inhibitor, BCL-xL PROTAC, pan-BCL inhibitor or ATR inhibitor, wherein the cancer is hepatocellular carcinoma (hepatoma), cholangiocarcinoma, colorectal carcinoma, small cell lung cancer, non-small cell lung cancer, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing’s tumor, leiomyosarcoma, rhabdomyosarcoma, pancreatic cancer, breast cancer, triple negative breast cancer, ovarian cancer, endometrial carcinoma, Fallopian tube cancer, prostate cancer, gastrointestinal stromal tumor (GIST), esophageal cancer, gallbladder cancer, gastrointestinal carcinoid tumor, duodenal cancer, gastroesophageal junction cancer, islet cell cancer, gastric cancer, anal cancer, cancer of the small intestine, pseudomyxoma peritonei, head and neck squamous cell carcinoma, Merkel cell carcinoma, tumor mutational burden-high cancer (TMB-H), microsatellite stable (MSS), mismatch repair proficient colon cancer, thyroid cancer, renal cell carcinoma, choriocarcinoma, seminoma, embryonal carcinoma, Wilms’ tumor, urothelial carcinoma, testicular cancer, bladder carcinoma, glioma, glioblastoma, multiforme, astrocytoma, medulloblastoma, craniopharyngioma, oligodendroglioma, malignant meningioma, diffuse intrinsic pontine glioma, melanoma, neuroblastoma, retinoblastoma, acute lymphoblastic B-cell leukemia, acute lymphoblastic T-cell leukemia, acute myeloblastic leukemia (AML), acute promyelocytic leukemia (APL), acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia (CML), chronic lymphocytic leukemia (CLL), hairy cell leukemia, multiple myeloma, lymphoblastic leukemia, myelogenous leukemia, lymphocytic leukemia, Hodgkin’s lymphoma, non-Hodgkin’s lymphoma, primary mediastinal large B-cell lymphoma, Waldenstrom’s macroglobulinemia, heavy chain disease, or polycythemia vera. In some embodiments, the cancer is colorectal cancer, cholangiocarcinoma, pancreatic cancer or ovarian cancer. In some embodiments, the non-small cell lung cancer is lung adenocarcinoma, lung squamous carcinoma, or lung large cell carcinoma. In some embodiments, the thyroid cancer is anaplastic thyroid cancer or follicular thyroid cancer. In some embodiments, the cancer is human epidermal growth factor receptor 2 (HER2)-positive. In some embodiments, the cancer is HER2-negative. In some embodiments, the cancer expresses a BRCA1 or BRCA2 gene oncogenic mutation. In some embodiments, the cancer does not express a BRCA1 or BRCA2 gene oncogenic mutation. In some embodiments, the methods for preventing, inhibiting, or reducing risk of metastasis of a cancer, comprise administering to a subject in need thereof an effective amount of LB-100 or a pharmaceutically acceptable salt thereof.

The present invention further provides methods for preventing, inhibiting, or reducing risk of metastasis of a cancer, comprising administering to a subject in need thereof an effective amount of: (a) LB-100 or an LB-100 ester, or a pharmaceutically acceptable salt thereof and (b) a WEE1 kinase inhibitor, CHK1 inhibitor, BCL-xL inhibitor, BCL-xL PROTAC or pan-BCL inhibitor, wherein the cancer is hepatocellular carcinoma (hepatoma), cholangiocarcinoma, colorectal carcinoma, small cell lung cancer, non-small cell lung cancer, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing’s tumor, leiomyosarcoma, rhabdomyosarcoma, pancreatic cancer, breast cancer, triple negative breast cancer, ovarian cancer, endometrial carcinoma, Fallopian tube cancer, prostate cancer, gastrointestinal stromal tumor (GIST), esophageal cancer, gallbladder cancer, gastrointestinal carcinoid tumor, duodenal cancer, gastroesophageal junction cancer, islet cell cancer, gastric cancer, anal cancer, cancer of the small intestine, pseudomyxoma peritonei, head and neck squamous cell carcinoma, Merkel cell carcinoma, tumor mutational burden-high cancer (TMB-H), microsatellite stable (MSS), mismatch repair proficient colon cancer, thyroid cancer, renal cell carcinoma, choriocarcinoma, seminoma, embryonal carcinoma, Wilms’ tumor, urothelial carcinoma, testicular cancer, bladder carcinoma, glioma, glioblastoma, multiforme, astrocytoma, medulloblastoma, craniopharyngioma, oligodendroglioma, malignant meningioma, diffuse intrinsic pontine glioma, melanoma, neuroblastoma, retinoblastoma, acute lymphoblastic B-cell leukemia, acute lymphoblastic T-cell leukemia, acute myeloblastic leukemia (AML), acute promyelocytic leukemia (APL), acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia (CML), chronic lymphocytic leukemia (CLL), hairy cell leukemia, multiple myeloma, lymphoblastic leukemia, myelogenous leukemia, lymphocytic leukemia, Hodgkin’s lymphoma, non-Hodgkin’s lymphoma, primary mediastinal large B-cell lymphoma, Waldenstrom’s macroglobulinemia, heavy chain disease, or polycythemia vera. In some embodiments, the cancer is colorectal cancer, cholangiocarcinoma, pancreatic cancer or ovarian cancer. In some embodiments, the non-small cell lung cancer is lung adenocarcinoma, lung squamous carcinoma, or lung large cell carcinoma. In some embodiments, the thyroid cancer is anaplastic thyroid cancer or follicular thyroid cancer. In some embodiments, the cancer is human epidermal growth factor receptor 2 (HER2)-positive. In some embodiments, the cancer is HER2-negative. In some embodiments, the cancer expresses a BRCA1 or BRCA2 gene oncogenic mutation. In some embodiments, the cancer does not express a BRCA1 or BRCA2 gene oncogenic mutation. In some embodiments, the methods for preventing, inhibiting, or reducing risk of metastasis of a cancer, comprise administering to a subject in need thereof an effective amount of LB-100 or a pharmaceutically acceptable salt thereof.

In some embodiments, the cancer is pancreatic cancer, breast cancer or ovarian cancer. In some embodiments, the pancreatic cancer, breast cancer or ovarian cancer expresses a BRCA1 or BRCA2 gene oncogenic mutation. In some embodiments, the pancreatic cancer, breast cancer or ovarian cancer does not express a BRCA1 or BRCA2 gene oncogenic mutation.

In some embodiments, the cancer is colorectal cancer. In some embodiments, the colorectal cancer expresses a KRAS, BRAF, PiK3CA, APC or p53 gene oncogenic mutation. In some embodiments, the colorectal cancer does not express a KRAS, BRAF, PiK3CA, APC or p53 gene oncogenic mutation. In some embodiments, the colorectal cancer is colon cancer. In some embodiments, the colon cancer is HER2-positive. In some embodiments, the colon cancer is HER2-negative. In some embodiments, the colorectal cancer is rectal cancer.

In some embodiments, the cancer is breast cancer. In some embodiments, the breast cancer is triple-negative breast cancer. In some embodiments, the breast cancer is HER2-positive breast cancer. In some embodiments, the breast cancer is HER2-negative breast cancer.

In some embodiments, the cancer is ovarian cancer. In some embodiments, the ovarian cancer is HER2-positive ovarian cancer. In some embodiments, the ovarian cancer is HER2 negative ovarian cancer.

In some embodiments, the cancer is pancreatic cancer. In some embodiments, the pancreatic cancer is HER2-positive pancreatic cancer. In some embodiments, the pancreatic cancer is HER2-negative pancreatic cancer. In some embodiments, the pancreatic cancer is pancreatic adenocarcinoma. In some embodiments, the pancreatic cancer is pancreatic ductal adenocarcinoma (PDAC).

In some embodiments, the cancer is bladder cancer, endometrial cancer, lung cancer, or head and neck cancer. In some embodiments, the bladder cancer, endometrial cancer, lung cancer, or head and neck cancer is HER2-positive. In some embodiments, the bladder cancer, endometrial cancer, lung cancer, or head and neck cancer is HER2-negative.

In some embodiments, the cancer is cholangiocarcinoma (CCA). In some embodiments, the CCA is bile duct cancer. In some embodiments, the CCA is intrahepatic, perihilar, or distal extrahepatic CCA. In some embodiments, the CCA is KRAS wildtype CCA. In some embodiments, the CCA expresses a KRAS gene oncogenic mutation.

In some embodiments of the methods of the invention, the cancer is microsatellite-stable (MSS). In some embodiments, the cancer is: microsatellite stable (MSS), mismatch repair proficient colon cancer; MSS, mismatch repair proficient triple-negative breast cancer; or MSS, mismatch repair proficient pancreatic cancer. In some embodiments, the cancer is microsatellite stable (MSS), mismatch repair proficient colorectal cancer. In some embodiments, the cancer is microsatellite stable (MSS), mismatch repair proficient colon cancer. In some embodiments, the cancer is microsatellite-instable (MSI). In some embodiments, the cancer is MSI and is more susceptible than MSS cancer to immune checkpoint blockade (ICB). In some embodiments, the MSS cancer is colorectal cancer, triple-negative breast cancer, or pancreatic cancer.

In some embodiments, the cancer has an amplified copy of MYC or has translocated MYC, e.g., C-MYC, N-MYC, or L-MYC. In some embodiments the cancer is associated with overexpression of MYC, i.e., C-MYC, N-MYC, or L-MYC.

In some embodiments, C-MYC amplification or overexpression causes, maintains or progresses the cancer. In some embodiments, the cancer caused, maintained or progressed by C-MYC amplification or overexpression is ovarian cancer, esophageal cancer, lung cancer, or breast cancer.

In some embodiments, N-MYC amplification causes, maintains or progresses the cancer. In some embodiments, the cancer caused, maintained or progressed by N-MYC amplification or overexpression is neuroblastoma, retinoblastoma, medulloblastoma, small cell lung cancer, or prostate cancer.

In some embodiments, L-MYC amplification or overexpression causes, maintains or progresses the cancer. In some embodiments, the cancer caused, maintained or progressed by L-MYC amplification is small cell lung cancer.

In some embodiments, the methods of the invention comprise administering to a subject an effective amount of: (a) LB-100 or a pharmaceutically acceptable salt thereof and (b) a WEE1 kinase inhibitor, CHK1 inhibitor, BCL-xL inhibitor, BCL-xL PROTAC pan-BCL inhibitor, or ATR inhibitor.

In some embodiments, the methods of the invention comprise administering to a subject an effective amount of: (a) LB-100 or a pharmaceutically acceptable salt thereof and (b) a WEE1 kinase inhibitor, CHK1 inhibitor, BCL-xL inhibitor, BCL-xL PROTAC or pan-BCL inhibitor.

In some embodiments, the methods of the invention comprise administering to a subject an effective amount of (a) LB-100.

In some embodiments, the methods of the invention comprise administering to a subject an effective amount of: (a) an LB-100 ester or a pharmaceutically acceptable salt thereof and (b) a WEE1 kinase inhibitor, CHK1 inhibitor, BCL-xL inhibitor, BCL-xL PROTAC, pan-BCL inhibitor or ATR inhibitor.

In some embodiments, the methods of the invention comprise administering to a subject an effective amount of: (a) an LB-100 ester or a pharmaceutically acceptable salt thereof and (b) a WEE1 kinase inhibitor, CHK1 inhibitor, BCL-xL inhibitor, BCL-xL PROTAC or pan-BCL inhibitor.

In some embodiments, the LB-100 ester has the structure:

| | |
|---|---|
| 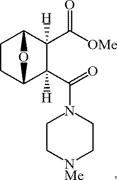 | 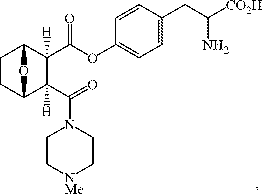 |
| 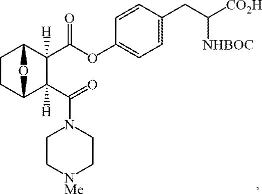 | 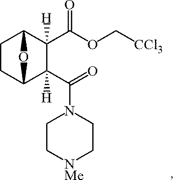 |
| 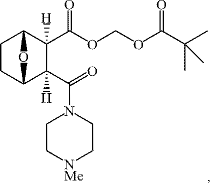 | 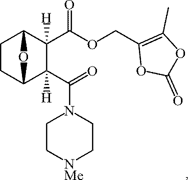 |
| 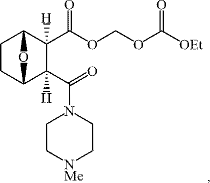 | 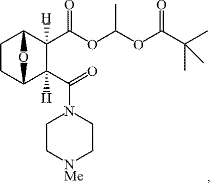 |
| 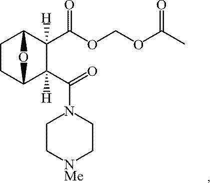 | 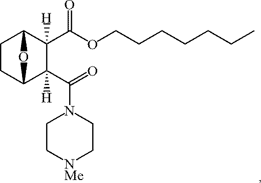 or |

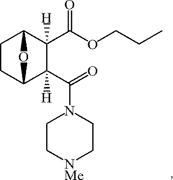

or a pharmaceutically acceptable salt thereof.

In some embodiments, the subject is an adult subject. In some embodiments, the subject is a pediatric subject.

In some embodiments, the LB-100 ester or pharmaceutically acceptable salt thereof is administered orally. In some embodiments, the LB-100 ester or pharmaceutically acceptable salt thereof is administered to the subject at a dose of 0.001 mg/kg body weight/day to 100 mg/kg body weight/day. In some embodiments, the LB-100 ester or pharmaceutically acceptable salt thereof is administered to the subject at a dose of about 0.003 mg/kg body weight/day to about 0.3 mg/kg body weight/day, e.g., about 0.003 mg/kg body weight/day, about 0.005 mg/kg body weight/day, about 0.010 mg/kg body weight/day, about 0.015 mg/kg body weight/day, about 0.020 mg/kg body weight/day, about 0.025 mg/kg body weight/day, about 0.030 mg/kg body weight/day, about 0.035 mg/kg body weight/day, about 0.040 mg/kg body weight/day, about 0.045 mg/kg body weight/day, about 0.050 mg/kg body weight/day, about 0.055 mg/kg body weight/day, about 0.060 mg/kg body weight/day, about 0.065 mg/kg body weight/day, about 0.070 mg/kg body weight/day, about 0.075 mg/kg body weight/day, about 0.080 mg/kg body weight/day, about 0.085 mg/kg body weight/day, about 0.090 mg/kg body weight/day, about 0.095 mg/kg body weight/day, about 0.10 mg/kg body weight/day, about 0.15 mg/kg body weight/day, about 0.20 mg/kg body weight/day, about 0.25 mg/kg body weight/day, or about 0.3 mg/kg body weight/day, including all values and subranges therebetween. In some embodiments, the LB-100 ester or pharmaceutically acceptable salt thereof is administered to the subject at a dose of about 0.03 mg/kg body weight/day to about 0.3 mg/kg body weight/day. In some embodiments, the LB-100 ester or pharmaceutically acceptable salt thereof is administered to the subject at a dose of about 0.1 mg/kg body weight/day to about 0.3 mg/kg body weight/day.

In some embodiments, LB-100 or a pharmaceutically acceptable salt thereof is administered intravenously. In some embodiments, the LB-100 or pharmaceutically acceptable salt thereof is administered intravenously as a component of a composition comprising monosodium glutamate or having a pH of about 10 to about 11. In some embodiments, the LB-100 or pharmaceutically acceptable salt thereof is administered intravenously as a component of a composition comprising monosodium glutamate and having a pH of about 10 to about 11. In some embodiments, the LB-100 or pharmaceutically acceptable salt thereof is administered intravenously as a component of a composition comprising monosodium glutamate or having a pH of 10-11. In some embodiments, the LB-100 or pharmaceutically acceptable salt thereof is administered intravenously as a component of a composition comprising monosodium glutamate and having a pH of 10-11.

In some embodiments, the LB-100 or pharmaceutically acceptable salt thereof is intravenously administered to the subject at a dose of about 0.1 mg/m² to about 5 mg/m². In some embodiments, the LB-100 or pharmaceutically acceptable salt thereof is intravenously administered to the subject at a dose of about 0.25 mg/m² to about 3.1 mg/m². In some embodiments, the LB-100 or pharmaceutically acceptable salt thereof is intravenously administered to the subject at a dose of about 1 mg/m² to about 3.1 mg/m². In some embodiments, the LB-100 or pharmaceutically acceptable salt thereof is intravenously administered to the subject at a dose of about 0.25 mg/m², about 0.5 mg/m², about 0.83 mg/m², about 1.25 mg/m², about 1.75 mg/m², about 2.33 mg/m², or about 3.1 mg/m². In some embodiments, the LB-100 or pharmaceutically acceptable salt thereof is intravenously administered to the subject at a dose of about 0.25 mg/m², about 0.5 mg/m², about 0.83 mg/m², about 1.25 mg/m², about 1.75 mg/m², about 2.33 mg/m², or about 3.1 mg/m².

In some embodiments, the LB-100 or LB-100 ester, or pharmaceutically acceptable salt thereof, is intravenously administered to the subject daily for a period of 6 weeks. In some embodiments, the LB-100 or LB-100 ester, or pharmaceutically acceptable salt thereof, is intravenously administered to the subject daily for a period of 3 weeks. In some embodiments, the LB-100 or LB-100 ester, or pharmaceutically acceptable salt thereof, is intravenously administered to the subject daily for 3 consecutive days, 4 consecutive days, or 5 consecutive days every 3 weeks. In some embodiments, the LB-100 or LB-100 ester, or pharmaceutically acceptable salt thereof, is intravenously administered to the subject daily for 3 consecutive days every 3 weeks.

In some embodiments, the methods of the invention comprise administering to a subject an effective amount of: (a) LB-100, an LB-100 ester or a pharmaceutically acceptable salt thereof and (b) a WEE1 kinase inhibitor, wherein the WEE1 kinase inhibitor is adavosertib, PD0166285, PD407824, ZN-c3, IMP7068, Debio 0123, SDGR2, SY4835, or NUV-569. In some embodiments, the WEE1 kinase inhibitor is PD0166285. In some embodiments, the WEE1 kinase inhibitor is adavosertib.

Accordingly, in some embodiments, the methods of the invention comprise administering to a subject in need thereof an effective amount of: (a) LB-100 or a pharmaceutically acceptable salt thereof and (b) a WEE1 kinase inhibitor, wherein the WEE1 kinase inhibitor is adavosertib.

In some embodiments, the WEE1 kinase inhibitor is administered to the subject at a dose of about 25 mg to about 500 mg, e.g., about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg. In some embodiments, the WEE1 kinase inhibitor is administered to the subject at a dose of about 50 mg to about 400 mg. In some embodiments, the WEE1 kinase inhibitor is administered to the subject at a dose of about 50 mg. In some embodiments, the WEE1 kinase inhibitor is administered to the subject at a dose of about 100 mg. In some embodiments, the WEE1 kinase inhibitor is administered to the subject at a dose of about 125 mg. In some embodiments, the WEE1 kinase inhibitor is administered to the subject at a dose of about 150 mg. In some embodiments, the WEE1 kinase inhibitor is administered to the subject at a dose of about 175 mg. In some embodiments, the WEE1 kinase inhibitor is administered to the subject at a dose of about 200 mg. In some embodiments, the WEE1 kinase inhibitor is administered to the subject at a dose of about 225 mg. In some embodiments, the WEE1 kinase inhibitor is administered to the subject at a dose of about 250 mg. In some embodiments, the WEE1 kinase inhibitor is administered to the subject at a dose of about 300 mg. In some embodiments, the WEE1 kinase inhibitor is administered to the subject at a dose of about 400 mg.

In some embodiments, the WEE1 kinase inhibitor is administered to the subject once per day (QD). In some embodiments, the WEE1 kinase inhibitor is administered to the subject twice per day (BID). In some embodiments, the WEE1 kinase inhibitor is administered to the subject once per day for 2 to 28 consecutive days. In some embodiments, the WEE1 kinase inhibitor is administered to the subject once per day for 2 to 28 consecutive days. In some embodiments, the WEE1 kinase inhibitor is administered to the subject once per day for 7, 14, 21, or 28 consecutive days. In some embodiments, the WEE1 kinase inhibitor is administered to the subject once per day for 21 consecutive days. In some embodiments, the WEE1 kinase inhibitor is administered to the subject once per day for 28 consecutive days. In some embodiments, the WEE1 kinase inhibitor is administered to the subject twice per day for 2 to 10 consecutive days. In some embodiments, the WEE1 kinase inhibitor is administered to the subject twice per day for 5 consecutive days. In some embodiments, the WEE1 kinase inhibitor is administered to the subject twice per day for 10 consecutive days.

In some embodiments, the WEE1 kinase inhibitor is administered according to the following schedule: (a) the WEE1 kinase inhibitor is administered to the subject twice per day for 1 day; (b) the WEE1 kinase inhibitor is not administered to the subject for one day immediately following the 1 day; (c) the WEE1 kinase inhibitor is administered to the subject twice per day for 1 consecutive day immediately following the one day; (d) the WEE1 kinase inhibitor is not administered to the subject for one day immediately following the 1 day; and (e) the WEE1 kinase inhibitor is administered to the subject twice per day for 1 day immediately following the one day.

In some embodiments, the WEE1 kinase inhibitor is administered according to the following schedule: (a) the WEE1 kinase inhibitor is administered to the subject once per day for 2 consecutive days; (b) the WEE1 kinase inhibitor is not administered to the subject for five days immediately following the 2 consecutive days; and (c) the WEE1 kinase inhibitor is administered to the subject once per day for 2 consecutive days immediately following the five days.

In some embodiments, the WEE1 kinase inhibitor is administered according to the following schedule: (a) the WEE1 kinase inhibitor is administered to the subject once per day for 2 consecutive days; (b) the WEE1 kinase inhibitor is not administered to the subject for five days immediately following the 2 consecutive days; (c) the WEE1 kinase inhibitor is administered to the subject once per day for 2 consecutive days immediately following the five days; (d) the WEE1 kinase inhibitor is not administered to the subject for five days immediately following the 2 consecutive days; and (e) the WEE1 kinase inhibitor is administered to the subject once per day for 2 consecutive days immediately following the five days.

In some embodiments, the WEE1 kinase inhibitor is administered according to the following schedule: (a) the WEE1 kinase inhibitor is administered to the subject twice per day for 2 consecutive days; and (b) the WEE1 kinase inhibitor is administered to the subject once per day for one days immediately following the 2 consecutive days.

In some embodiments, the WEE1 kinase inhibitor is administered according to the following schedule: (a) the WEE1 kinase inhibitor is administered to the subject twice per day for 2 consecutive days; (b) the WEE1 kinase inhibitor is administered to the subject once per day for one day immediately following the 2 consecutive days; (c) the WEE1 kinase inhibitor is not administered to the subject for four days immediately following the 1 day; (d) the WEE1 kinase inhibitor is administered to the subject twice per day for 2 consecutive days immediately following the 4 days; (e) the WEE1 kinase inhibitor is administered to the subject once per day for one day immediately following the 2 consecutive days; (f) the WEE1 kinase inhibitor is not administered to the subject for four days immediately following the 1 day; (g) the WEE1 kinase inhibitor is administered to the subject twice per day for 2 consecutive days immediately following the 4 days; and (h) the WEE1 kinase inhibitor is administered to the subject once per day for one day immediately following the 2 consecutive days.

In some embodiments, the WEE1 kinase inhibitor is administered according to the following schedule: (a) the WEE1 kinase inhibitor is administered to the subject once per day for 3 consecutive days; and (b) the WEE1 kinase inhibitor is not administered to the subject for 4 days immediately following the 3 consecutive days.

In some embodiments, the WEE1 kinase inhibitor is administered according to the following schedule: (a) the WEE1 kinase inhibitor is administered to the subject twice per day for 3 consecutive days; (b) the WEE1 kinase inhibitor is not administered to the subject for four days immediately following the 3 consecutive days; (c) the WEE1 kinase inhibitor is administered to the subject twice per day for 3 consecutive days immediately following the four days; (d) the WEE1 kinase inhibitor is not administered to the subject for four days immediately following the 3 consecutive days; and (e) the WEE1 kinase inhibitor is administered to the subject twice per day for 3 consecutive days immediately following the four days.

In some embodiments, the WEE1 kinase inhibitor is administered according to the following schedule: (a) the WEE1 kinase inhibitor is administered to the subject twice per day for 3 consecutive days; (b) the WEE1 kinase inhibitor is not administered to the subject for four days immediately following the 3 consecutive days; and (c) the WEE1 kinase inhibitor is administered to the subject twice per day for 3 consecutive days immediately following the four days.

In some embodiments, the WEE1 kinase inhibitor is administered according to the following schedule: (a) the WEE1 kinase inhibitor is administered to the subject once per day for 5 consecutive days; (b) the WEE1 kinase inhibitor is not administered to the subject for two days immediately following the 5 consecutive days; and (c) the WEE1 kinase inhibitor is administered to the subject once per day for 5 consecutive days immediately following the two days.

In some embodiments, the WEE1 kinase inhibitor is administered according to the following schedule: (a) the WEE1 kinase inhibitor is administered to the subject once per day for 5 consecutive days; (b) the WEE1 kinase inhibitor is not administered to the subject for nine days immediately following the 5 consecutive days; and (c) the WEE1 kinase inhibitor is administered to the subject once per day for 5 consecutive days immediately following the nine days.

In some embodiments, the WEE1 kinase inhibitor is administered orally. In some embodiments, the WEE1 kinase inhibitor is administered intravenously.

In some embodiments, the methods of the invention comprise administering to a subject an effective amount of: (a) LB-100, an LB-100 ester or a pharmaceutically acceptable salt thereof and (b) a CHK1 inhibitor, wherein the CHK1 inhibitor is GDC-0575, prexasertib, rabusertib, SCH-900776, CCT-245737, AZD-7762, PF-477736, GDC-0425, or SRA737. In some embodiments, the CHK1 inhibitor is rabusertib. In some embodiments, the CHK1 inhibitor is prexasertib.

In some embodiments, the CHK1 inhibitor is administered to the subject at a dose of about 5 mg to about 100 mg, e.g., about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg. In some embodiments, the CHK1 inhibitor is administered to the subject at a dose of about 10 mg to about 50 mg. In some embodiments, the CHK1 inhibitor is administered to the subject at a dose of about 5 mg. In some embodiments, the CHK1 inhibitor is administered to the subject at a dose of about 10 mg. In some embodiments, the CHK1 inhibitor is administered to the subject at a dose of about 15 mg. In some embodiments, the CHK1 inhibitor is administered to the subject at a dose of about 20 mg. In some embodiments, the CHK1 inhibitor is administered to the subject at a dose of about 25 mg. In some embodiments, the CHK1 inhibitor is administered to the subject at a dose of about 30 mg. In some embodiments, the CHK1 inhibitor is administered to the subject at a dose of about 35 mg. In some embodiments, the CHK1 inhibitor is administered to the subject at a dose of about 40 mg. In some embodiments, the CHK1 inhibitor is administered to the subject at a dose of about 45 mg. In some embodiments, the CHK1 inhibitor is administered to the subject at a dose of about 50 mg.

In some embodiments, the CHK1 inhibitor is rabusertib. In some embodiments, the CHK1 inhibitor is rabusertib and the rabusertib is administered to the subject at a dose of about 150 mg to about 500 mg of rabusertib, e.g., about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg. In some embodiments, the rabusertib is administered to the subject at a dose of about 150 mg to about 250 mg of rabusertib. In some embodiments, the rabusertib is administered to the subject at a dose of about 170 mg or about 230 mg of rabusertib.

In some embodiments, the CHK1 inhibitor is prexasertib. In some embodiments, the prexasertib is intravenously administered to the subject, e.g., at a dose of about 50 mg/m² to about 150 mg/m², e.g., about 50 mg/m², about 55 mg/m², about 60 mg/m², about 65 mg/m², about 70 mg/m², about 75 mg/m², about 80 mg/m², about 85 mg/m², about 90 mg/m², about 95 mg/m², about 100 mg/m², about 105 mg/m², about 110 mg/m², about 115 mg/m², about 120 mg/m², about 125 mg/m², about 130 mg/m², about 135 mg/m², about 140 mg/m², about 145 mg/m², or about 150 mg/m². In some embodiments, the prexasertib is intravenously administered to the subject at a dose of about 75 mg/m² to about 100 mg/m² of prexasertib. In some embodiments, the prexasertib is intravenously administered to the subject at a dose of about 80 mg/m².

In some embodiments, the CHK1 inhibitor is SCH-900776. In some embodiments, the SCH-900776 is intravenously administered to the subject, e.g., at a dose of about 10 mg/m² to about 500 mg/m². In some embodiments, the SCH-900776 is intravenously administered to the subject at a dose of about 10 mg/m² to about 200 mg/m², e.g., about 10 mg/m², about 20 mg/m², about 30 mg/m², about 40 mg/m², about 50 mg/m², about 60 mg/m², about 70 mg/m², about 80 mg/m², about 90 mg/m², about 100 mg/m², about 110 mg/m², about 120 mg/m², about 130 mg/m², about 140 mg/m², about 150 mg/m², about 160 mg/m², about 170 mg/m², about 180 mg/m², about 190 mg/m², or about 200 mg/m². In some embodiments, the SCH-900776 is intravenously administered to the subject at a dose of about 10 mg/m², about 20 mg/m², about 40 mg/m², about 80 about mg/m², about 112 mg/m², about 150 mg/m² or about 200 mg/m² to the subject.

In some embodiments, the CHK1 inhibitor is PF-00477736. In some embodiments, the PF-00477736 is intravenously administered to the subject, e.g., at a dose of about 250 mg/m² to about 1250 mg/m². In some embodiments, the PF-00477736 is administered to the subject at a dose of about 750 mg/m² to about 1250 mg/m², e.g., about 750 mg/m², 800 mg/m², 850 mg/m², 900 mg/m², 950 mg/m², 1000 mg/m², 1050 mg/m², 1100 mg/m², 1150 mg/m², 1200 mg/m², or about 1250 mg/m².

In some embodiments, the CHK1 inhibitor is GDC-0575. In some embodiments, the GDC-0575 is intravenously administered to the subject.

In some embodiments, the CHK1 inhibitor is administered to the subject once per day (QD). In some embodiments, the CHK1 inhibitor is administered to the subject twice per day (BID). In some embodiments, the CHK1 inhibitor is administered to the subject once per week. In some embodiments, the CHK1 inhibitor is administered to the subject once every two weeks. In some embodiments, the CHK1 inhibitor is administered to the subject for two, three, four or five consecutive days every week. In some embodiments, the CHK1 inhibitor is administered to the subject for three consecutive days every week. In some embodiments, the CHK1 inhibitor is administered to the subject for four consecutive days every week. In some embodiments, the CHK1 inhibitor is administered to the subject for five consecutive days every week.

In some embodiments, the CHK1 inhibitor is administered according to the following schedule: (a) the CHK1 inhibitor is administered to the subject once per day for a first one day; (b) the CHK1 inhibitor is not administered to the subject for a first six days, the first six days immediately following the first one day; (c) the CHK1 inhibitor is administered to the subject once per day for a second one day, the second one day immediately following the first six days; (d) the CHK1 inhibitor is not administered to the subject for a second six days, the second six days immediately following the second one day; and (e) the CHK1 inhibitor is administered to the subject once per day for a third one day.

In some embodiments, the methods of the invention comprise administering to a subject an effective amount of: (a) LB-100, an LB-100 ester or a pharmaceutically acceptable salt thereof and (b) a BCL-xL inhibitor.

In some embodiments, the BCL-xL inhibitor is A-1155463. In some embodiments, the A-1155463 is administered to the subject at a dose of about 25 mg to about 500 mg, e.g., about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg, including all ranges and values therebetween.

In some embodiments, the methods of the invention comprise administering to a subject an effective amount of: (a) LB-100, an LB-100 ester or a pharmaceutically acceptable salt thereof and (b) a BCL-xL PROTAC.

In some embodiments, the BCL-xL PROTAC is DT2216 or PZ15227. In some embodiments, the DT2216 or PZ15227 is administered to the subject at a dose of about 25 mg to about 500 mg, e.g., about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg, including all ranges and values therebetween.

In some embodiments, the methods of the invention comprise administering to a subject an effective amount of: (a) LB-100, an LB-100 ester or a pharmaceutically acceptable salt thereof and (b) pan-BCL inhibitor.

In some embodiments, the pan-BCL inhibitor is navitoclax. In some embodiments, the navitoclax is administered to the subject at a dose of about 25 mg to about 500 mg, e.g., about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg, including all ranges and values therebetween. In some embodiments, the navitoclax is administered to the subject at a dose of about 100 mg to about 400 mg. In some embodiments, the navitoclax is administered to the subject at a dose of about 150 mg to about 325 mg. In some embodiments, the navitoclax is administered to the subject at a dose of about 150 mg or about 325 mg.

In some embodiments, the navitoclax is administered to the subject once per day (QD). In some embodiments, the navitoclax is administered to the subject once per day for a period of 1 to 5 weeks. In some embodiments, the navitoclax is administered to the subject once per day for one week. In some embodiments, the navitoclax is administered to the subject once per day for 2 weeks. In some embodiments, the navitoclax is administered to the subject once per day for 3 weeks. In some embodiments, the navitoclax is administered to the subject once per week. In some embodiments, the navitoclax is administered to the subject once every two weeks. In some embodiments, the navitoclax is administered to the subject once every three weeks. In some embodiments, the navitoclax is administered to the subject once every four weeks.

In some embodiments, the methods of the invention comprise administering to a subject an effective amount of: (a) LB-100, an LB-100 ester or a pharmaceutically acceptable salt thereof and (b) an ATR inhibitor.

In some embodiments, the ATR inhibitor is administered to the subject at a dose of about 25 mg to about 500 mg, e.g., about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg, including all ranges and values therebetween. In some embodiments, the ATR inhibitor is administered to the subject at a dose of about 115 mg to about 450 mg. In some embodiments, the ATR inhibitor is berzosertib, and the berzosertib is administered to the subject at a dose of about 150 mg to about 450 mg. In some embodiments, the ATR inhibitor is berzosertib, and the berzosertib is administered to the subject at a dose of about 325 mg to about 375 mg.

In some embodiments of the methods of the invention, LB-100 or an LB-100 ester, or a pharmaceutically acceptable salt thereof, is administered to the subject in need of cancer treatment in a range from about 1 mg to about 1000 mg or any amount ranging from and to these values. In some embodiments, the LB-100 or an LB-100 ester, or a pharmaceutically acceptable salt thereof, is administered to the subject in need thereof in a range from about 1 mg to about 900 mg, about 1 mg to about 800 mg, about 1 mg to about 700 mg, about 1 mg to about 600 mg, about 1 mg to about 500 mg, about 1 mg to about 400 mg, or about 1 mg to about 300 mg.

In some embodiments of the methods of the invention, the LB-100 or LB-100 ester, or a pharmaceutically acceptable salt thereof, is administered to the subject in need of cancer treatment in a daily dose ranging from about 1 mg to about 1000 mg or any amount ranging from and to these values. In some embodiments, the LB-100 or LB-100 ester, or a pharmaceutically acceptable salt thereof, is administered to the subject in need thereof at a daily dose of about 1000 mg, about 950 mg, about 900 mg, about 850 mg, about 800 mg, about 750 mg, about 700 mg, about 650 mg, about 600 mg, about 550 mg, about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, or about 1 mg.

In some embodiments of the methods of the invention, the LB-100 or LB-100 ester, or a pharmaceutically acceptable salt thereof, is administered to the subject in need thereof once a day at a dose of about 1 mg to about 1000 mg or any amount ranging from and to these values.

In some embodiments of the methods of the invention, the LB-100 or LB-100 ester, or a pharmaceutically acceptable salt thereof, is administered to the subject in need thereof twice a day, each dose of the LB-100 or an LB-100 ester, or a pharmaceutically acceptable salt thereof, in about 1 mg to about 500 mg or any amount ranging from and to these values. In some embodiments, the LB-100 or LB-100 ester, or a pharmaceutically acceptable salt thereof, is administered to the subject in need thereof twice a day, each dose of the LB-100 or LB-100 ester, or a pharmaceutically acceptable salt thereof, being about 500 mg, about 450 mg, about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, or about 1 mg.

In some embodiments of the methods of the invention, the LB-100 or LB-100 ester, or a pharmaceutically acceptable salt thereof, is administered to the subject in need of cancer treatment three times a day, each dose of the LB-100 or LB-100 ester, or a pharmaceutically acceptable salt thereof, in about 1 mg to about 400 mg or any amount ranging from and to these values. In some embodiments, the LB-100 or LB-100 ester, or a pharmaceutically acceptable salt thereof, is administered to the subject in need of cancer treatment three times a day, each dose of the LB-100 or LB-100 ester, or a pharmaceutically acceptable salt thereof, being about 400 mg, about 350 mg, about 300 mg, about 250 mg, about 200 mg, about 150 mg, about 100 mg, about 80 mg, about 60 mg, about 40 mg, about 20 mg, about 10 mg, about 5 mg, or about 1 mg.

In some embodiments, the subject is cancer-treatment naïve. In some embodiments, the subject is not cancer-treatment naïve.

In some embodiments, the subject is PP2A inhibitor-treatment naïve. In some embodiments, the subject is PP2A inhibitor-treatment naïve, wherein the PP2A inhibitor is LB-100 or an LB-100 ester, or pharmaceutically acceptable salt thereof.

In some embodiments, the subject is checkpoint inhibitor-treatment naïve. In some embodiments, the subject is checkpoint inhibitor-treatment naive, wherein the checkpoint inhibitor is a CHK1 inhibitor. In some embodiments, the subject is CHK1 inhibitor-treatment naïve, wherein the CHK1 inhibitor is CGDC-0575, prexasertib, rabusertib, SCH-900776, CCT-245737, AZD-7762, PF-477736, GDC-0425 or SRA737.

In some embodiments, the subject is tyrosine kinase inhibitor-treatment naïve. In some embodiments, the subject is tyrosine kinase inhibitor-treatment naive, wherein the tyrosine kinase inhibitor is a WEE1 kinase inhibitor. In some embodiments, the subject is WEE1 kinase inhibitor-treatment naive, wherein the WEE1 kinase inhibitor is adavosertib, PD0166285, PD407824, ZN-c3, IMP7068, Debio 0123, SDGR2, SY4835, or NUV-569.

In some embodiments, the subject is BCL-xL inhibitor-treatment naïve. In some embodiments, the subject is BCL-xL inhibitor-treatment naive, wherein the BCL-xL inhibitor is A-1155463.

In some embodiments, the subject is pan-BCL inhibitor-treatment naïve. In some embodiments, the subject is pan-BCL inhibitor-treatment naive, wherein the pan-BCL inhibitor is navitoclax.

In some embodiments, the subject is BCL-xL PROTAC-treatment naïve. In some embodiments, the subject is BCL-xL PROTAC-treatment naive, wherein the BCL-xL PROTAC is DT2216 or PZ15227.

In some embodiments, the subject is ATR inhibitor-treatment naïve. In some embodiments, the subject is ATR inhibitor-treatment naive, wherein the ATR inhibitor is berzosertib (M6620), camonsertib (RP-3500), ceralasertib (AZD6738), elimusertib (BAY1895344), dactolisib, SKLB-197, AZ20, VE-821, VX-803, ETP-46464, ATG-018, schisandrin B, CGK 733, torin 2, or HAMNO.

In some embodiments, the subject is not PP2A inhibitor-treatment naïve. In some embodiments, the subject is not PP2A inhibitor-treatment naive, wherein the PP2A inhibitor is LB-100, an LB-100 ester, or pharmaceutically acceptable salt thereof.

In some embodiments, the subject is not checkpoint inhibitor-treatment naïve. In some embodiments, the subject is not checkpoint inhibitor-treatment naive, wherein the checkpoint inhibitor is a CHK1 inhibitor. In some embodiments, the subject is not CHK1 inhibitor-treatment naïve, wherein the CHK1 inhibitor is CGDC-0575, prexasertib, rabusertib, SCH-900776, CCT-245737, AZD-7762, PF-477736, GDC-0425 or SRA737.

In some embodiments, the subject is not tyrosine kinase inhibitor-treatment naïve. In some embodiments, the subject is not tyrosine kinase inhibitor-treatment naive, wherein the tyrosine kinase inhibitor is a WEE1 kinase inhibitor. In some embodiments, the subject is not WEE1 kinase inhibitor-treatment naive, wherein the WEE1 kinase inhibitor is adavosertib, PD0166285, PD407824, ZN-c3, IMP7068, Debio 0123, SDGR2, SY4835, or NUV-569.

In some embodiments, the subject is not BCL-xL inhibitor-treatment naïve. In some embodiments, the subject is not BCL-xL inhibitor-treatment naive, wherein the BCL-xL inhibitor is A-1155463.

In some embodiments, the subject is not pan-BCL inhibitor-treatment naïve. In some embodiments, the subject is not pan-BCL inhibitor-treatment naive, wherein the pan-BCL inhibitor is navitoclax.

In some embodiments, the subject is not BCL-xL PROTAC-treatment naïve. In some embodiments, the subject is not BCL-xL PROTAC-treatment naive, wherein the BCL-xL PROTAC is DT2216 or PZ15227.

In some embodiments, the subject is not ATR inhibitor-treatment naïve. In some embodiments, the subject is not ATR inhibitor-treatment naive, wherein the ATR inhibitor is berzosertib (M6620), camonsertib (RP-3500), ceralasertib (AZD6738), elimusertib (BAY1895344), dactolisib, SKLB-197, AZ20, VE-821, VX-803, ETP-46464, ATG-018, schisandrin B, CGK 733, torin 2, or HAMNO.

In some embodiments of the methods of the invention, the WEE1 kinase inhibitor, CHK1 inhibitor, BCL-xL inhibitor, BCL-xL PROTAC, pan-BCL inhibitor or ATR inhibitor is administered concurrently with, prior to, or after administering the LB-100 or LB-100 ester, or a pharmaceutically acceptable salt thereof. In some embodiments of the methods of the invention, the WEE1 kinase inhibitor, CHK1 inhibitor, BCL-xL inhibitor, BCL-xL PROTAC or pan-BCL inhibitor is administered concurrently with, prior to, or after administering the LB-100 or LB-100 ester, or a pharmaceutically acceptable salt thereof. In some embodiments, the WEE1 kinase inhibitor, CHK1 inhibitor, or BCL inhibitor is administered concurrently with, prior to, or after administering the LB-100 or LB-100 ester, or pharmaceutically acceptable salt thereof.

In some embodiments, the methods of the invention comprise administering to a subject in need of cancer treatment an effective amount of (a) LB-100 or an LB-100 ester, or a pharmaceutically acceptable salt thereof, and (b) a WEE1 kinase inhibitor, CHK1 inhibitor, BCL-xL inhibitor, BCL-xL PROTAC, pan-BCL inhibitor or ATR inhibitor set forth in a Composition of Tables D1-D3.

In some embodiments of the methods of the invention, administration of (a) LB-100 or an LB-100 ester, or a pharmaceutically acceptable salt thereof, and (b) a WEE1 kinase inhibitor, CHK1 inhibitor, BCL-xL inhibitor, BCL-xL PROTAC, pan-BCL inhibitor or ATR inhibitor is synergistic and more effective for treating cancer, or for preventing, inhibiting, or reducing risk of metastasis of a cancer, than administration of (a) in the absence of (b), or administration of (b) in the absence of (a).

In some embodiments of the methods of the invention, administration of (a) LB-100 or an LB-100 ester, or a pharmaceutically acceptable salt thereof, and (b) a WEE1 kinase inhibitor, CHK1 inhibitor, BCL-xL inhibitor, BCL-xL PROTAC or pan-BCL inhibitor is synergistic and more effective for treating cancer, or for preventing, inhibiting, or reducing risk of metastasis of a cancer, than administration of (a) in the absence of (b), or administration of (b) in the absence of (a).

In some embodiments of the methods of invention, administration of (a) LB-100 or an LB-100 ester, or a pharmaceutically acceptable salt thereof, and (b) a WEE1 kinase inhibitor, CHK1 inhibitor, BCL-xL inhibitor, BCL-xL PROTAC, pan-BCL inhibitor or ATR inhibitor, activate mitogenic signaling. In some embodiments of the methods of invention, administration of (a) LB-100 or an LB-100 ester, or a pharmaceutically acceptable salt thereof, and (b) a WEE1 kinase inhibitor, CHK1 inhibitor, BCL-xL inhibitor, BCL-xL PROTAC, pan-BCL inhibitor or ATR inhibitor, sensitizes cells of a cancer disclosed herein to a WEE1 kinase inhibitor, CHK1 inhibitor, BCL-xL inhibitor, BCL-xL PROTAC, pan-BCL inhibitor or ATR inhibitor, disclosed herein, that targets stress response pathways (e.g., metabolic stress, proteotoxic stress, mitotic stress, oxidative stress, or DNA damage stress).

In some embodiments of the methods of invention, administration of (a) LB-100 or an LB-100 ester, or a pharmaceutically acceptable salt thereof, and (b) a WEE1 kinase inhibitor, CHK1 inhibitor, BCL-xL inhibitor, BCL-xL PROTAC, pan-BCL inhibitor or ATR inhibitor, reduces the subject’s likelihood of experiencing an adverse event, compared to administration of (a) in the absence of (b), or administration of (b) in the absence of (a), wherein the adverse event is fatigue, increased blood creatinine level, increased aspartate aminotransferase level, headache, hypernatremia, hypoalbumenia, nausea, proteinuria, pyrexia, increase alanine aminotransferase level, constipation, peripheral neuropathy, peripheral edema, sinus tachycardia, absominal discomfort, abdominal distension, accelerated hypertension, anemia, arthralgia, increased blood alkaline phosphatase level, increased blood urea level, candidiasis, chest pain, chills, decreased appetite, dermatitis acneiform, diarrhea, dizziness, decreased ejection fraction, QT prolongation, gait disturbance, gastrointestinal disorder, generalized edema, gingival pain, hypercalcemia, hyperkalemia, hypertension, hypoesthesia, hypokinesia, hypotension, hypoxia, insomnia, mucosal inflammation, muscle twitching muscular weakness, neutropenia, edema, skin pain, peripheral sensory neuropathy, decreased platelet count, pleural effusion, tachypnea, tremor, vomiting, weight loss, creatinine renal clearance, dyspnea, hyponeutremia, or decreased lymphocyte count.

### Other Pharmaceutically Active Agents

In some embodiments, the methods of the invention further comprise administering to the subject an effective amount of another pharmaceutically active agent.

In some embodiments, the other pharmaceutically active agent is an immunotherapeutic agent. In some embodiments, the immunotherapeutic agent is dostarlimab-gxly, ticilimumab, avelumab, pembrolizumab, avelumab, durvalumab, nivolumab, cemiplimab, ABX196, sintilimab, camrelizumab, spartalizumab, toripalimab, bispecific antibody XmAb20717, mapatumumab, tremelimumab, carotuximab, tocilizumab, ipilimumab, atezolizumab, bevacizumab, ramucirumab, IBI305, ascrinvacumab, TCR T-cell therapy agent, cytokine-based biologic agent IRX-2, bempegaldesleukin, DKN-01, PTX-9908, AK104, PT-112, SRF388, ET1402L1-CART, Glypican 3-specific Chimeric Antigen Receptor Expressing T Cells (CAR-T cells), CD147-targeted CAR-T cells, NKG2D-based CAR T-cells, or neoantigen reactive T cells.

In some embodiments the other pharmaceutically active agent is a chemotherapeutic agent. In some embodiments, the chemotherapeutic agent is altretamine, dendmustine, busulfan, carboplatin, chlorambucil, cisplatic, cyclophosphamide, dacarbazine, ifosamide, lomustine, mechlorethamine, melphalan, oxaliplatin, temozolomide, thiotepa, trabectedin, streptozocin, azacytidine, 5-fluorouracil (5-FU), 6-mercaptopurine (6-MP), capecitabine, cladribine, clofarabine, cytarabine, decitabine, floxuridine, fludarabine, gemcitabine, hydroxyurea, methotrexate, nelarabine, pemetrexed, pentostatin, pralatrexate, thioguanine, trifluridine/tipiracil, daunorubicin, doxorubicin, epirubicin, idarubicin, valrubicin, bleomycin, dactinomycin, mitomycin C, mitoxantrone, irinotecan, topotecan, etoposide, teniposide, cabizitaxel, docetaxel, nab-paclitaxel, paclitaxel, vinblastine, vincristine, vinorelbine, eribulin, ixabepilone, mitotane, omacetaxine, procarbazine, romidepsin, vorinostat, prednisone, methylprednisone, dexamethasone, tamoxifen, sitravatinib or leuprolide.

In some embodiments, the methods of the invention further comprise administering radiation therapy to the subject. In some embodiments, the radiation therapy is gamma ray radiation therapy or x-ray radiation therapy. In some embodiments, the radiation therapy is administered via a gamma ray or x-ray radiation apparatus.

In some embodiments, the radiation therapy is administered concurrently with, prior to or subsequent to the administration of (a) LB-100 or an LB-100 ester, or a pharmaceutically acceptable salt thereof, or (b) a WEE1 kinase inhibitor, CHK1 inhibitor, BCL-xL inhibitor, BCL-xL PROTAC, pan-BCL inhibitor or ATR inhibitor. In some embodiments, the radiation therapy is administered concurrently with, prior to or subsequent to the administration (a) LB-100 or an LB-100 ester, or a pharmaceutically acceptable salt thereof, and (b) a WEE1 kinase inhibitor, CHK1 inhibitor, BCL-xL inhibitor, BCL-xL PROTAC, pan-BCL inhibitor or ATR inhibitor.

### EXAMPLES

### Biological Assays

### Cell Culture

Cell lines were cultured in RPMI medium, supplemented with 10% FBS, 1% penicillin/streptomycin and 2 mM L-glutamine. All cell lines were cultured at 37° C. in 5% CO₂. All cell lines were validated by STR profiling. Mycoplasma tests were performed every 2-3 months.

### Dose-Response Assays

Drug-response assays were performed in triplicate, using black-walled 384-well plates. Cells were plated with a 20% density (approximately) and incubated for approximately 24 hours to allow attachment to the plate. Drugs were then added to the cells using the Tecan D300e digital dispenser. 10 µM phenylarsine oxide was used as positive control (0% cell viability) and DMSO was used as negative control (100% cell viability). 3-5 days later, culture medium was removed and resazurin was added to the plates. After 1-4 hours incubation (depending on the cell line), fluorescence (560_{Ex}/590_{Em}) was recorded using the EnVision (Perkin Elmer).

### IncuCyte-Based Proliferation and Caspase 3/7 Activity

IncuCyte assays were performed in triplicate, using black-walled 96-well plates. Cells were plated at a very low density. Plates were then placed in the in the IncuCyte ZOOM, which imaged the cells every 4 h. Approximately 24 h after plating drugs were added to the cells using the Tecan D300e digital dispenser, as indicated. Phase-contrast images were collected and analyzed to detect cell proliferation based on confluence.

In some cases, as indicated, IncuCyte® Caspase-3/7 green apoptosis assay reagent (Essen Bioscience 4440) was also added to the culture medium. Here, green fluorescent images were also collected and analyzed (by dividing the detected green fluorescence confluence by the phase-contrast confluence) to detect caspase 3/7 activity.

### Crystal Violet Long-Term Viability Assays

Cells were plated at a very low density in 6-well plates and incubated for approximately 24 h to allow attachment to the plates. Drugs were then added to the cells using the Tecan D300e digital dispenser, as indicated. The culture media/drugs were refreshed every 2-3 days. When control wells (DMSO) were confluent cells were fixed using a solution of 2% formaldehyde (Millipore 104002) diluted in phosphate-buffered saline (PBS). Two hours later, they were stained, using a solution of 0.1% crystal violet (Sigma HT90132) diluted in water. No more than 10 min later the staining solution was removed, plates were washed with water and left to dry overnight. Finally, plates were scanned and stored.

### Western Blot Analysis

After the indicated culture period and drug treatment, cells were washed with cold PBS, then lysed with RIPA buffer (25 mM Tris-HCl, pH 7.6, 150 mM NaCl, 1% NP-40, 1% sodium deoxycholate, 0.1% SDS) containing Complete Protease Inhibitor cocktail and phosphatase inhibitor cocktails II and III. Samples were then centrifuged for 10 min at 15,000 x g at 4° C. and supernatant was collected. Protein concentration of the samples was normalized after performing a Bicinchoninic Acid (BCA) assay. Protein samples (denatured with DTT followed by 5 min heating at 95° C.) were then loaded in a 4-12% polyacrylamide gel. Gels were run (SDS-PAGE) for approximately 45 min at 175 volts. Proteins were transferred from the gel to a polyvinylidene fluoride (PVDF) membrane at 330 mA for 90 min. After the transfer, membranes were incubated in blocking solution (5% bovine serum albumin (BSA) in PBS with 0.1% Tween-20 (PBS-T)). Subsequently, membranes were probed with primary antibody in blocking solution (1:1000) overnight at 4° C. Membranes were then washed 3 times for 10 min with PBS-T, followed by 1 h incubation at room temperature with the secondary antibody (HRP conjugated, 1:10,000) in blocking solution. Membranes were again washed 3 times for 10 min in PBS-T. Finally, a chemiluminescence substrate (ECL, Bio-Rad) was added to the membranes and the signal was imaged using the ChemiDoc-Touch (Bio-Rad)

### CRISPR-KO Screens

The appropriate number of cells to achieve a 250-fold representation of the Brunello library for all the screen arms and replicates were transduced at approximately 50% confluence in the presence of polybrene (8 µg/mL) with the appropriate volume of the lentiviral-packaged sgRNA library. Cells were incubated overnight, followed by replacement of the lentivirus-containing medium with fresh medium containing puromycin (2 µg/mL). The lentivirus volume to achieve a MOI of 0.3, as well as the puromycin concentration to achieve a complete selection in 3 days was previously determined for each cell line. After puromycin selection, cells were split into the indicated arms/replicates (for each arm, the appropriate number of cells to keep a 250-fold representation of the library was plated at approximately 10-20% confluence) and a T0 (reference) time point was harvested. Cells were maintained as indicated. In case a passage was required, cells were reseeded at the appropriate number to keep at least a 500-fold representation of the library. Cells (enough to keep at least a 500-fold representation of the library, to account for losses during DNA extraction) were collected when indicated, washed with PBS, pelleted and stored at -80° C. until DNA extraction.

### DNA Extraction, PCR Amplification and Illumina Sequencing

Genomic DNA (gDNA) was extracted (Zymo Research, D3024) from cell pellets according to the manufacturer’s instructions. For every sample, gDNA was quantified and the necessary DNA to maintain a 250-fold representation of the library was used for subsequent procedures (for this, an assumption was made that that each cell contains 6.6 pg genomic DNA). Each sample was divided over 50 µl PCR reactions (using a maximum of 1 µg gDNA per reaction) using barcoded forward primers to be able to deconvolute multiplexed samples after next generation sequencing. PCR mixture per reaction: 10 µl 5x HF Buffer, 1 µl 10 µM forward primer, 1 µl 10 µM reverse primer, 0.5 µl Phusion polymerase (Thermo Fisher, F-530XL), 1 µl 10 mM dNTPs, adding H2O and template to 50 µl. Cycling conditions: 30 sec at 98° C., 20× (30 sec at 98° C., 30 sec at 60° C., 1 min at 72° C.), 5 min at 72° C. The products of all reactions from the same sample were pooled and 2 µl of this pool was used in a subsequent PCR reaction using primers containing adapters for next generation sequencing. The same cycling protocol was used, this time for 15 cycles. Next, PCR products were purified using the ISOLATE II PCR and Gel Kit. DNA concentrations were measured, and based on this, samples were equimolarly pooled and subjected to Illumina next generation sequencing (HiSeq 2500 High Output Mode, Single-Read, 65 bp). Mapped read-counts were subsequently used as input for the further analyses.

### Bioinformatics Analysis

For each CRISPR screen the sgRNA count data for each sample was normalized for sequence depth using DESeq2, with the difference that the median instead of the total value of a sample was used. The results from the DESeq2 analysis were sorted using the DESeq2 statistic in decreasing order putting the most enriched sgRNA at the top. MAGeCK Robust Rank (RRA) was then used to test for enrichment of the sgRNAs of a gene towards the top for which RRA will generate a multiple testing corrected p-value (FDR).

### Example 1. Anti-Proliferative Effects of LB-100 in Various Colorectal Cancer Cell Lines

To evaluate the ability of LB-100 to sensitize cancer cells to stress-targeted drugs, a panel of seven colorectal cancer cell lines having diverse oncogenic driver mutations was chosen (Table 1).

TABLE 1

Mutation background of the colorectal cancer panel.

| | | | | | | | | |
|---|---|---|---|---|---|---|---|---|
| Call Line | Disease | MSI status | KRASmut | BRAFmut | PiK3CAmut | APCmut | p53mut | Other |
| SW48G | Colon adenocardnoma | MSS | G12V | - | - | Yes | Yes | - |
| HT29 | Colon adenocardnoma | MSS | - | V500E | P449T | Yes | Yes | SMAD4 |
| DlFi | Rectal carcinoma | MSS | - | - | - | Yes | Yes | EGFR amplification |
| HCT116 | Colon carcinoma | MSI | G13D | - | H1047R | - | - | ACVR2A, BRCA2, CTNN51, CDKN2A, EP300 and TGFBR2 |
| LoVo | Colon adenocardnoma | MSI | G13D | - | - | Yes | - | ACVR2A, B2M, F5XW7, 5MAD2, TGFBR2 |
| RKO | Colon carcinoma | MSI | | V500E | H1047R | - | - | ACVR2A, TGFBR2 |
| DLD1 | Colon adenocardnoma | MSI | G13D | - | Yes | Yes | Yes | ACVR2A, B2M, EP300, TGFBR2 |

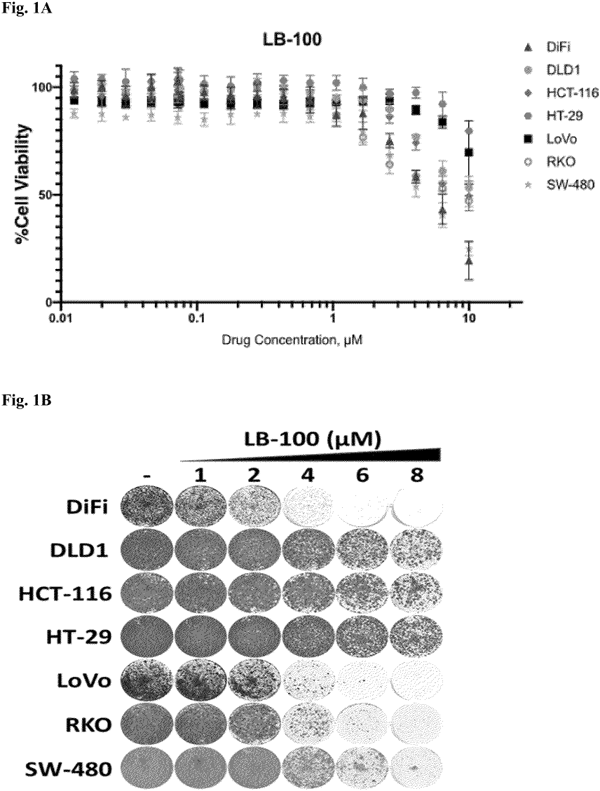

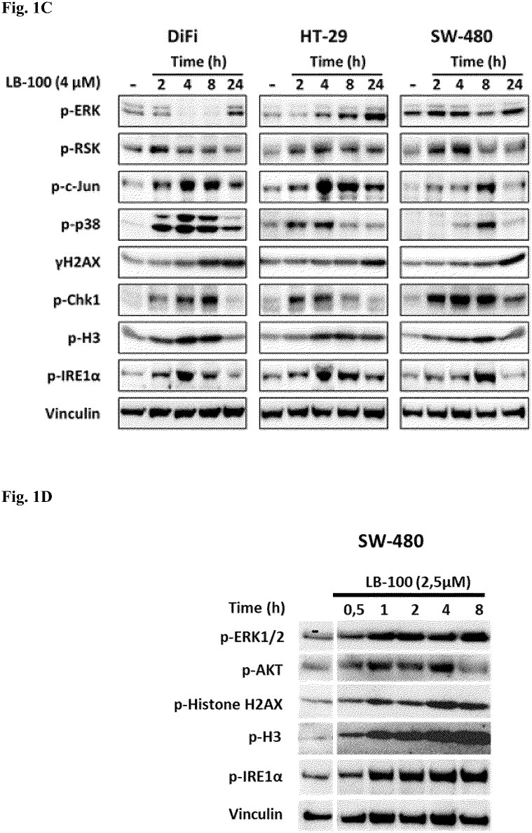

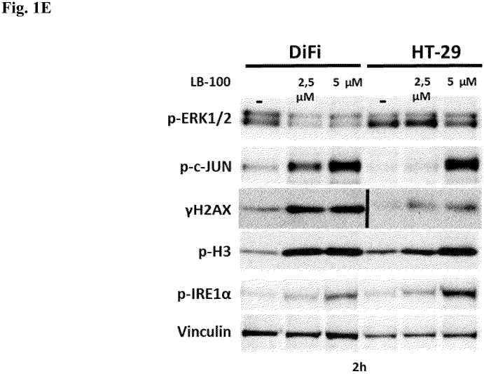

The effect of increasing doses of LB-100 to the Table 1 cell lines, both in short-term cell viability assays (FIG. 1A) and long-term cell proliferation assays (FIG. 1B), was analyzed. For the short-term study, cells were cultured with increasing concentrations of LB-100 for 5 days. Then the cell viability was measured using resazurin. For the longer-term study, cells were grown in the absence or presence of LB-100 at the indicated concentrations for 7 days, then fixed and stained. The results showed that LB-100 was cytotoxic across the different cell lines of the panel, with moderate toxicity in the lower micro molar range both in the short-term and long-term assays. Time-course Western blots from DiFi, HT-29, and SW-480 cells using vinculin as a control confirmed that LB-100 activates mitogenic signaling pathways and engages stress response pathways in these cancer cell lines (FIG. 1C, FIG. 1D, and FIG. 1E), although the intensity and kinetics of such activations vary among these cells. An increase in phospho-p38, phospho-CHK1, phospho-histone H3 and phospho IRE1α in the cell lines was also observed.

### Example 2. Identification of Stress-Targeted Drugs for Combination With LB-100 that Kill Colorectal Cancer Cells

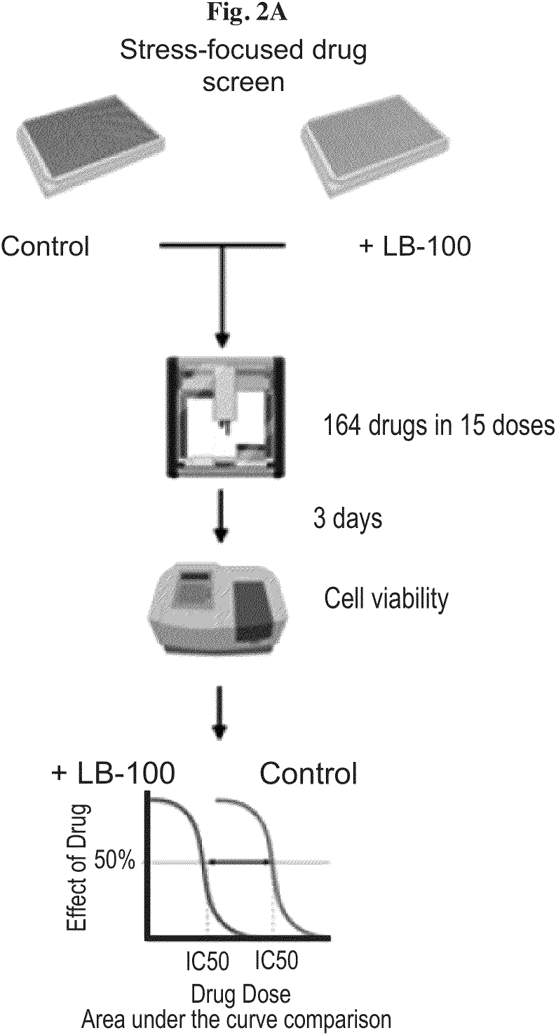

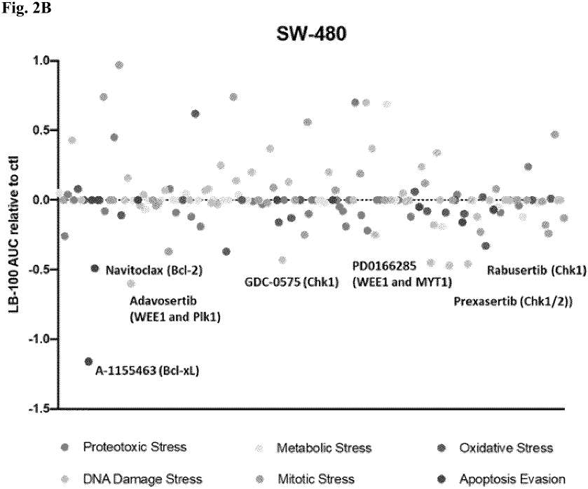

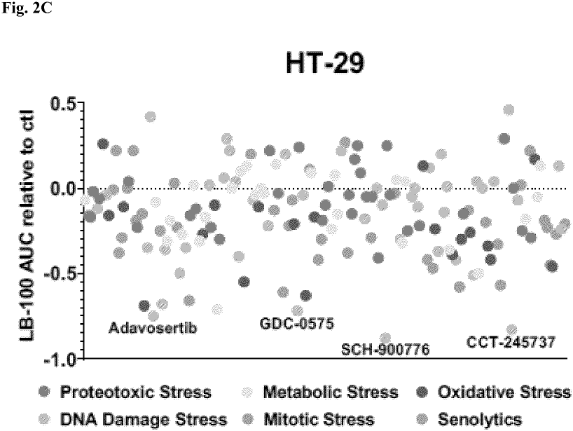

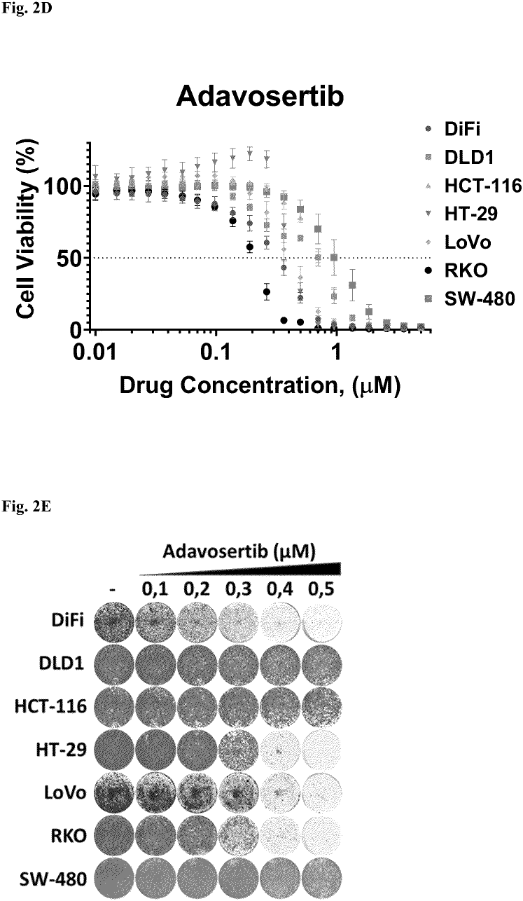

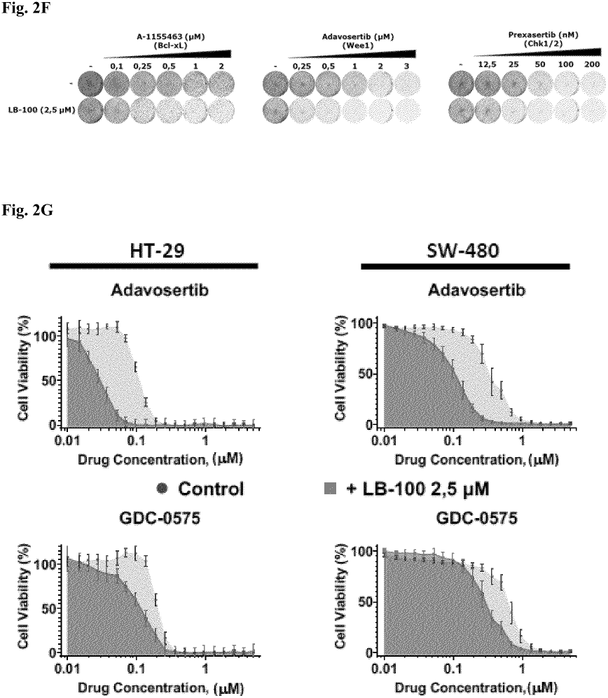

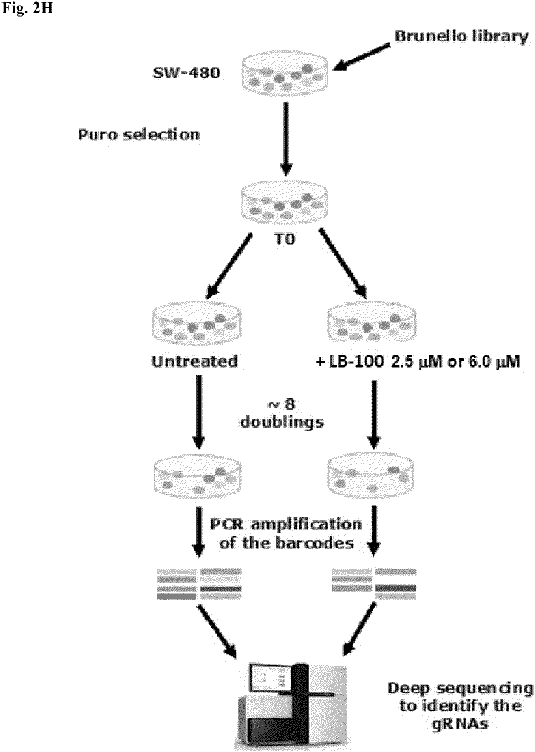

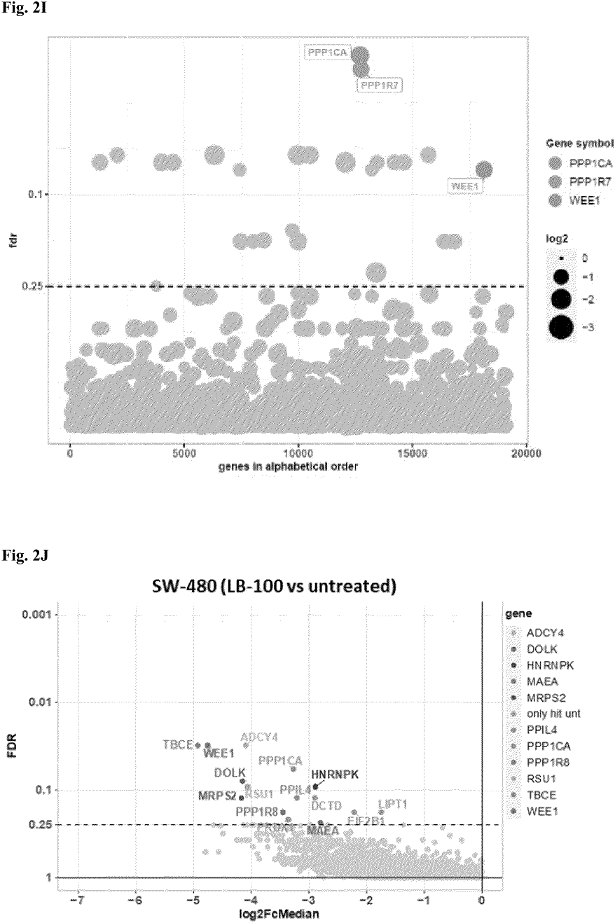

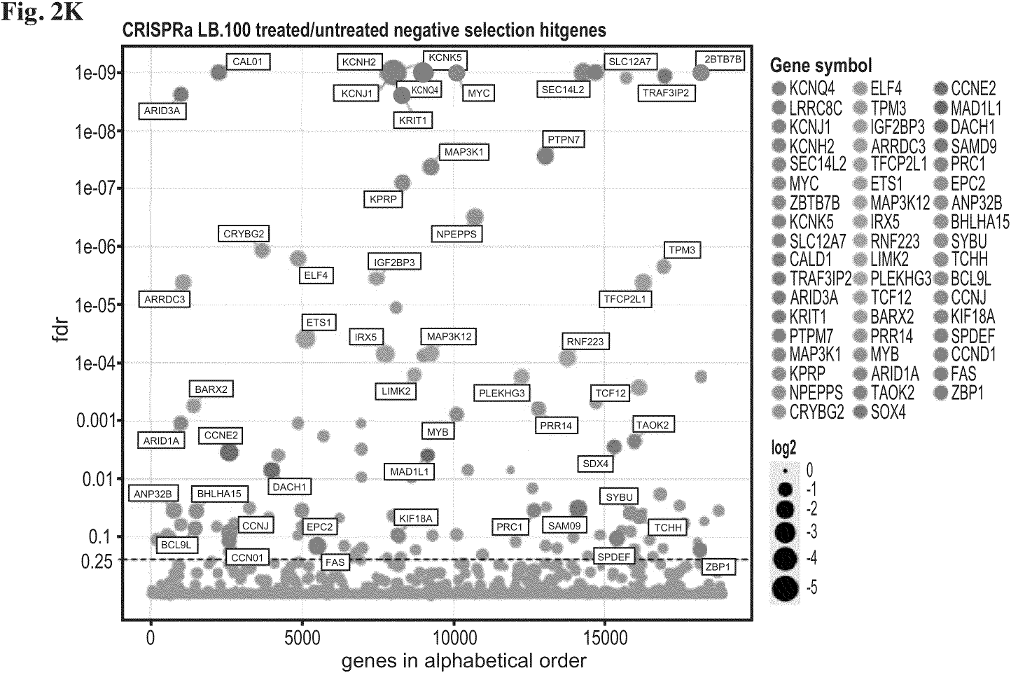

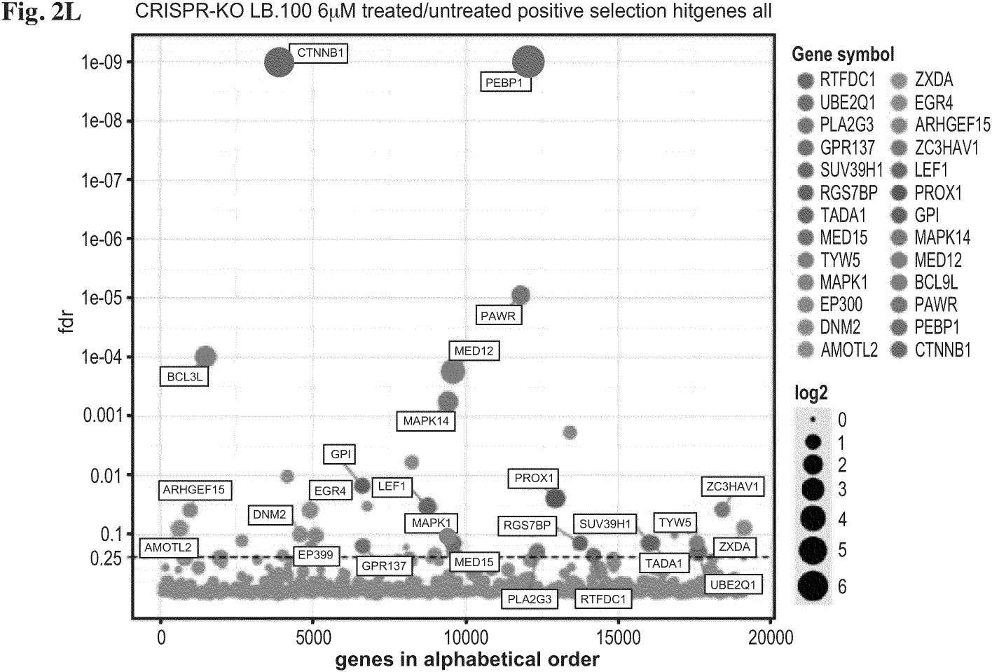

To identify stress-targeted drugs that can be combined with LB-100 to kill cancer cells, a library of 164 compounds was built targeting the different stress responses associated with a malignant phenotype (i.e., proteotoxic stress, oxidative stress, DNA damage stress, mitotic stress, metabolic stress, and apoptosis evasion). These compounds were investigated in 15 different concentrations using a literature-based concentration range in order to obtain dose-response curves for each of these drugs. A schematic outline of the stress-focused screen is shown in FIG. 2A. Using SW-480 and HT-29 cells, and cell viability after three days as a readout, the normalized area under the curve (AUC) was compared in the presence or absence (control) of a sub-lethal concentration of LB-100 (2.5 µM) for each the 164 compounds in the screening library. The results, compiled in FIG. 2B, show that the WEE1 kinase inhibitors adavosertib and PD0166285, and the CHK1 inhibitors GDC-0575, prexasertib, and rabusertib are among the bottom 5% normalized AUCs of the screened compounds, indicating that their toxicity was increased in the presence of LB-100 in SW-480 cells. The toxicities of the BCL-xL inhibitor A-1155463 and the pan-BCL inhibitor navitoclax were also increased in the presence of LB-100 (FIG. 2B). Similar results were obtained using HT-29 cells, where it was found that LB-100 increased the toxicity of adavosertib and GDC-0575 (FIG. 2C).

The stress-focused drug screen also showed that CCT-245737 and SCH-900776 exhibit increased toxicity to HT-29 cells in the presence of a sub-lethal concentration of LB-100, and rabusertib and prexasertib exhibit increased toxicity to SW-480 cells in the presence of a sub-lethal concentration of LB-100.

Based on these results, A-1155463, adavosertib, and prexasertib were selected for follow-up validations. Long-term cell proliferation assays, where cells were grown in the absence or presence of LB-100 and the indicated drugs at multiple concentrations for 10-14 days, then fixed and stained, confirmed the increased toxicity of these drugs when combined with LB-100 in SW-480 cells (FIG. 2F).

Using GDC-0575 as CHK1 inhibitor and adavosertib as a WEE1 kinase inhibitor, the addition of LB-100 increased the cytotoxicity of both drugs in a panel of CRC cells (FIG. 2G). Thus, the stress-focused drug screens identified CHK1 or WEE1 inhibition as a vulnerability of CRC cells treated with LB-100.

In an unbiased investigation of potential vulnerabilities of cells treated with LB-100, a genome-wide CRISPR screen (FIG. 2H) was carried out in SW-480 cells with the same sub-lethal concentration of LB-100 used in the stress-focused drug screens. This was done to identify genes whose depletion show synthetic lethality with LB-100. From this study, it was found that gRNAs targeting 17 genes were significantly depleted in the LB-100-treated samples compared to the untreated controls (FIG. 21). Among these genes are two components of the major Ser/Thr phosphatase protein phosphatase 1 (PP1): the catalytic (PPP1CA) and one regulatory subunit (PPP1R7), indicating an increased dependence on PP1 activity upon PP2A inhibition. Consistent with the compound screen, gRNAs targeting WEE1 were also significantly depleted from LB-100-treated samples compared to the untreated controls (FIG. 2I). These data indicate that in the presence of LB-100, these cells become more dependent on WEE1 expression and provide an unbiased validation of the toxicity resulting from the combination of LB-100 and WEE1 inhibition in colorectal cancer cells.

### Example 3. Synergistic Effect of LB-100 With Adavosertib and Prexasertib in Colorectal Cancer (CRC) Cells

### LB-100 Is Synergistic With Adavosertib in CRC Cancer Cell Lines

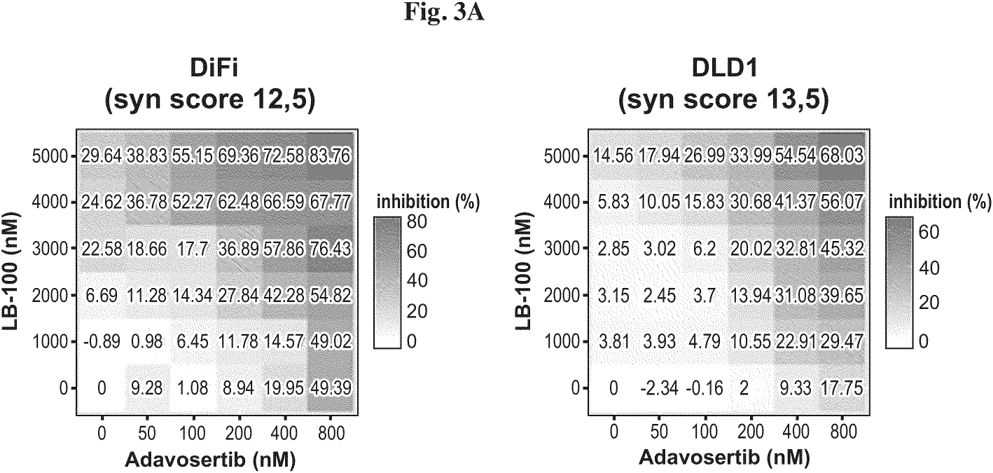

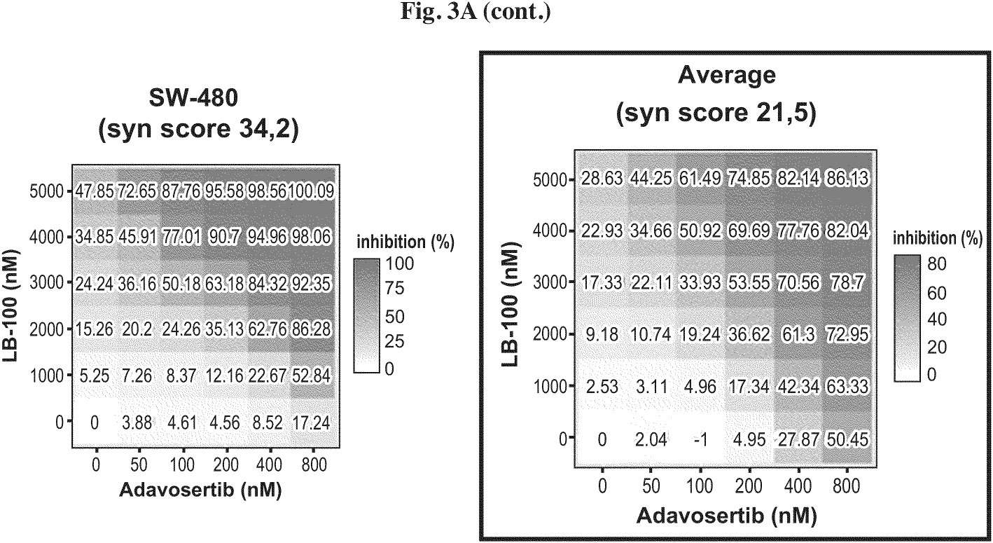

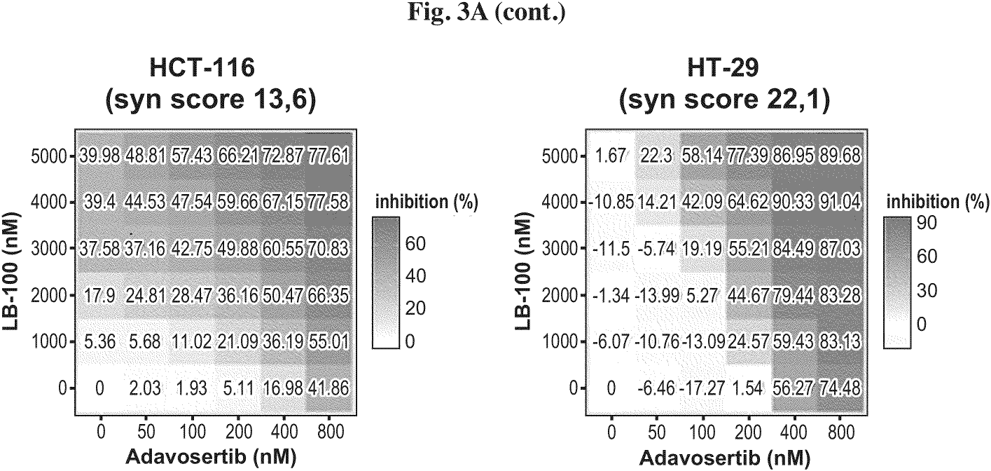

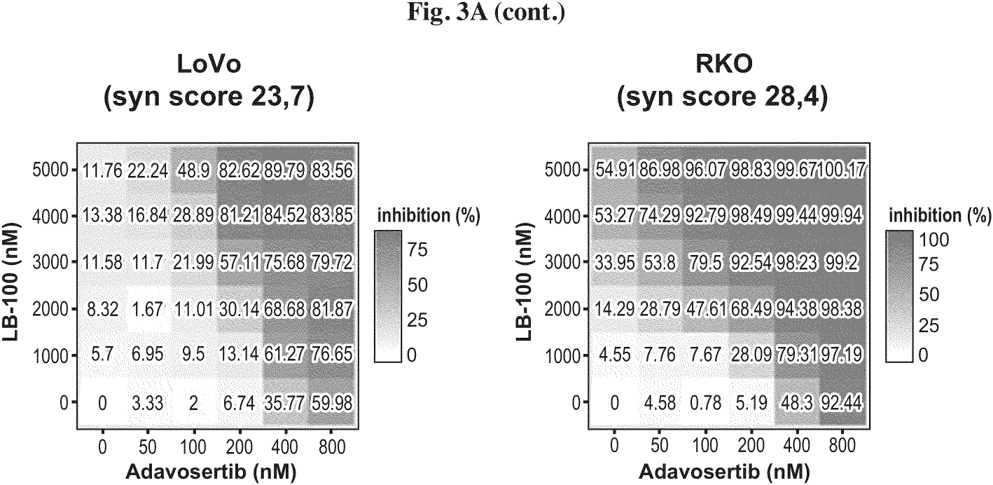

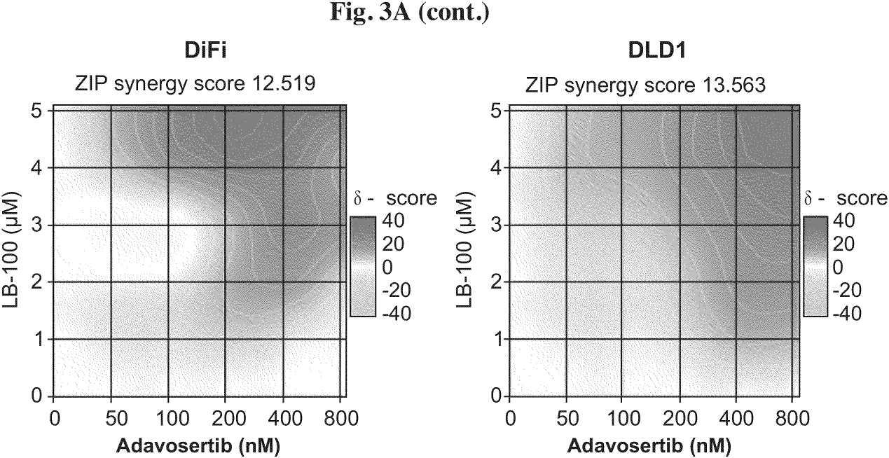

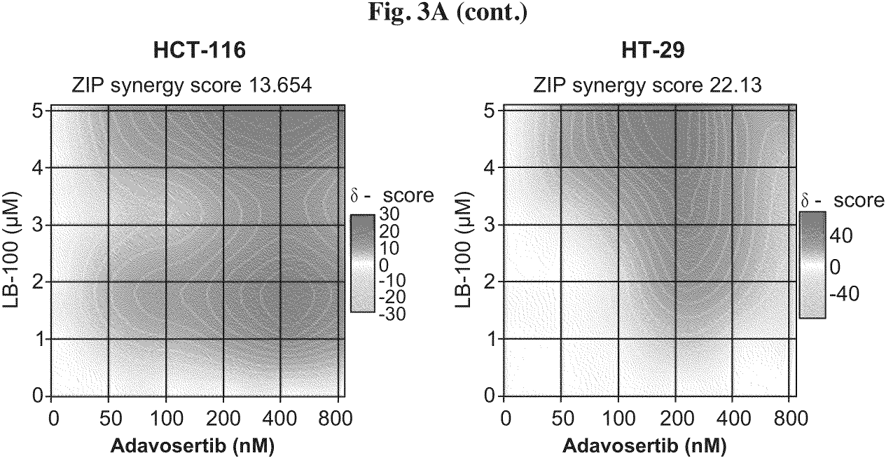

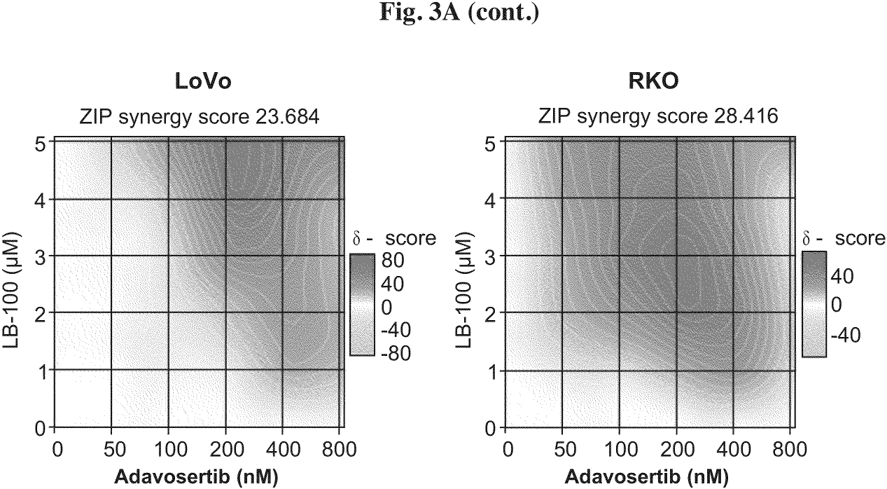

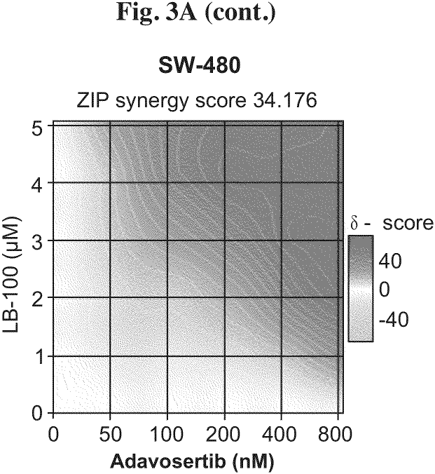

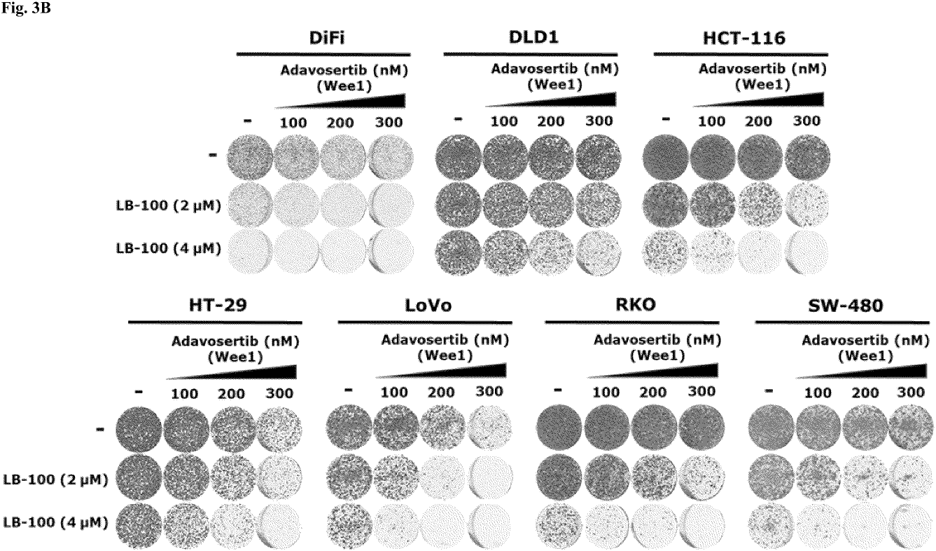

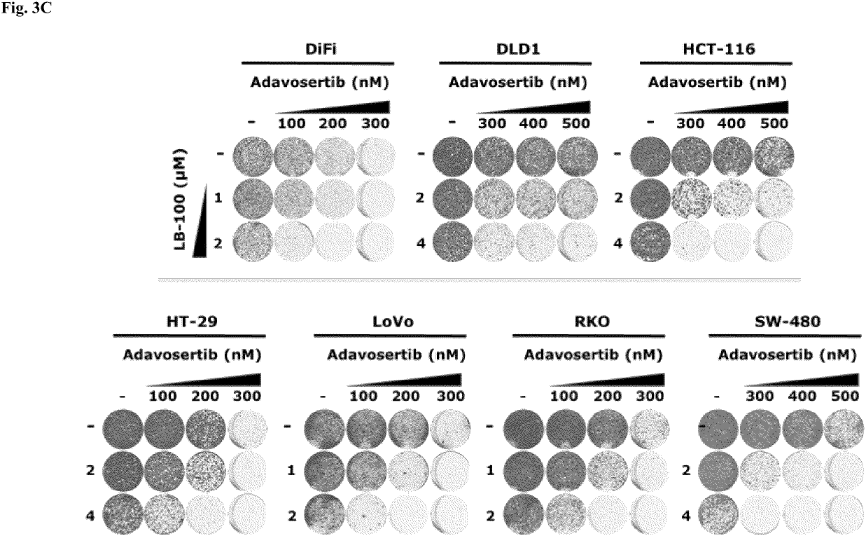

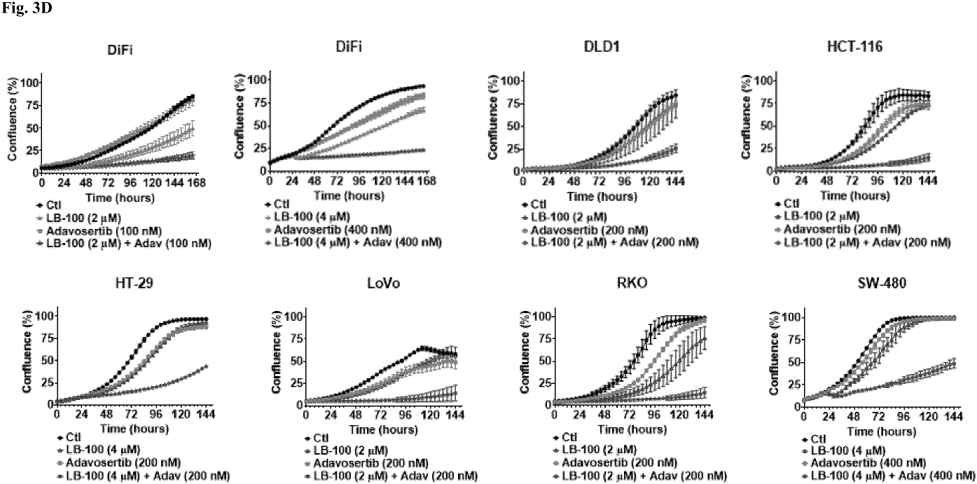

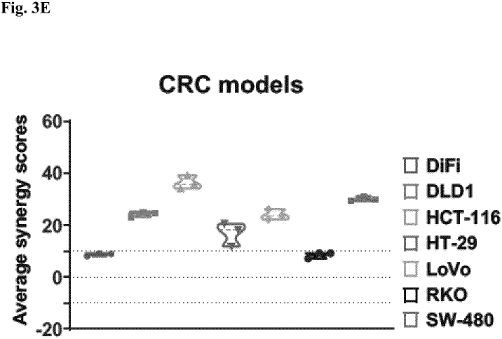

Adavosertib was used as the WEE1 kinase inhibitor to investigate the effect of the combination with LB-100 in the panel of CRC cells. Dose-response curves for this drug indicated IC50s ranging from about 0.18 to 1 µM across the panel (FIG. 2D). Toxicity of the combination in long-term viability assays was assessed. The cells were cultured for at least ten days and the drugs were refreshed every other day. The toxicity of the single drugs in this experimental setup was assessed. Variable toxicity of both drugs across the panel (FIG. 1B and FIG. 2E) was found, as anticipated by the drug-response curves. Informed by the toxicity of the single drugs, how sublethal concentrations of each drug would increase the overall toxicity in combination was addressed. The results indicate strong toxicity of the combination in concentrations for which the single drugs show, at best, a modest effect (FIG. 3B and FIG. 3C). It is noteworthy that DLD1, HCT-116, and SW-480 were largely tolerant to up to 500 nM of adavosertib, but such tolerance was abolished in the combination with LB-100 (FIG. 3C). The combination toxicity of LB-100 and adavosertib was further confirmed across the CRC panel by IncuCyte-based short-term cell proliferation assay. Colorectal cancer cells were plated and incubated overnight to allow attachment to the plate. Cells were then treated at the indicated conditions and confluence over time was measured by IncuCyte®, FIG. 3D).

The combination of multiple concentrations of LB-100 and adavosertib was evaluated to determine if a synergistic effect is observed in a panel of colorectal cancer cell lines. In the synergy studies, cells were cultured with the indicated concentrations of LB-100 and adavosertib for 5 days, then the cell viability was measured using resazurin. The relative inhibition of cell viability is shown for each pair of concentrations. Synergy scores (ZIP) were calculated using the SynergyFinder 2.0 online tool. A score above 10 indicates synergy. Average synergy across the panel is highlighted at the bottom right box. The matrices in FIG. 3A show LB-100 and adavosertib synergy in all seven colorectal cell lines, with an average synergy score of 21.5 across the panel. Long-term cell proliferation assays corroborate these findings, showing increased toxicity of the combination as compared to the single drugs in the seven colorectal cancer cells (FIG. 3B and FIG. 3C). In another synergy study, the range of doses investigated was expanded to further confirm that the two drugs act synergistically in the panel of CRC cells. Synergy matrices combining 5 doses of each drug showed toxicities larger than expected based on the effect of the single drugs, as indicated by the respective synergy scores (FIG. 3E). DiFi and RKO cells showed synergy scores slightly below the proposed threshold of 10, which is consistent with their higher sensitivity to adavosertib as a single drug (FIG. 2D). These results confirm the synergistic effects of LB-100 and adavosertib in a diverse set of CRC cell lines, indicating that this drug synergy is not critically dependent on a specific set of oncogenic driver mutations in colorectal cancer.

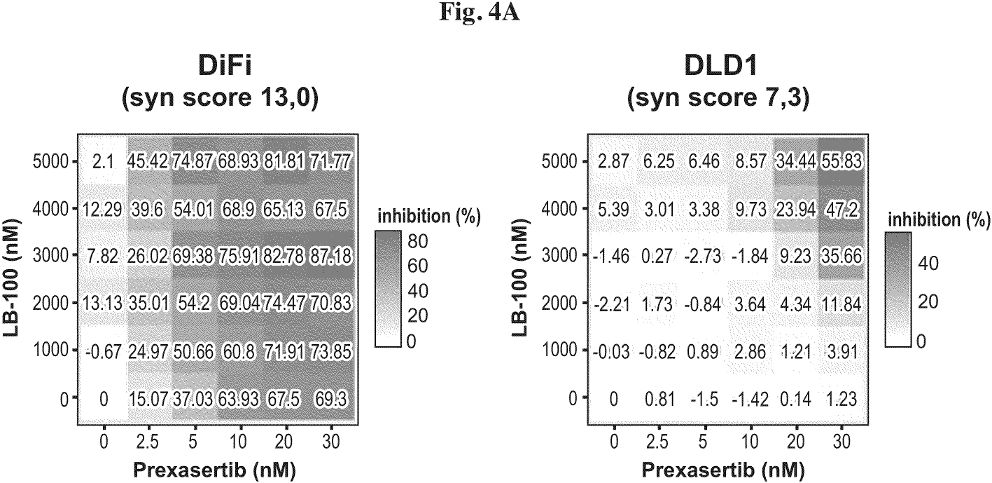

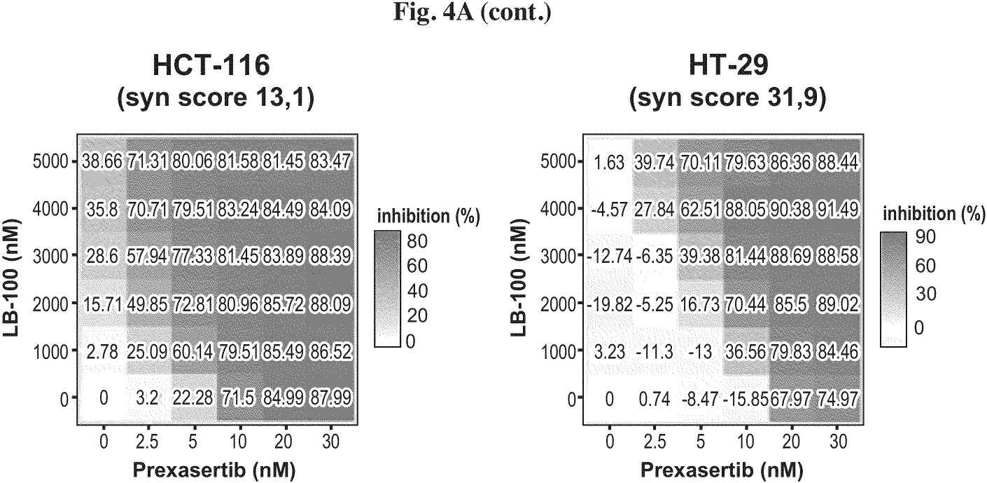

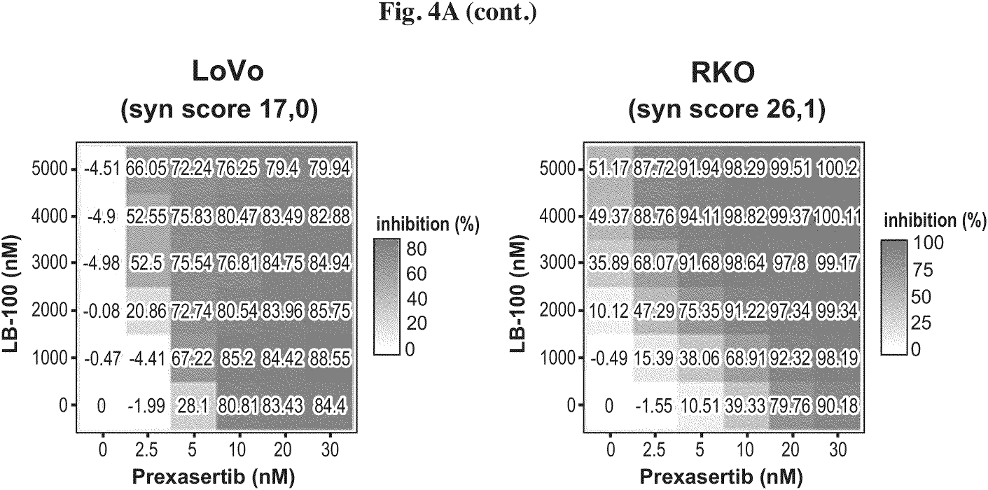

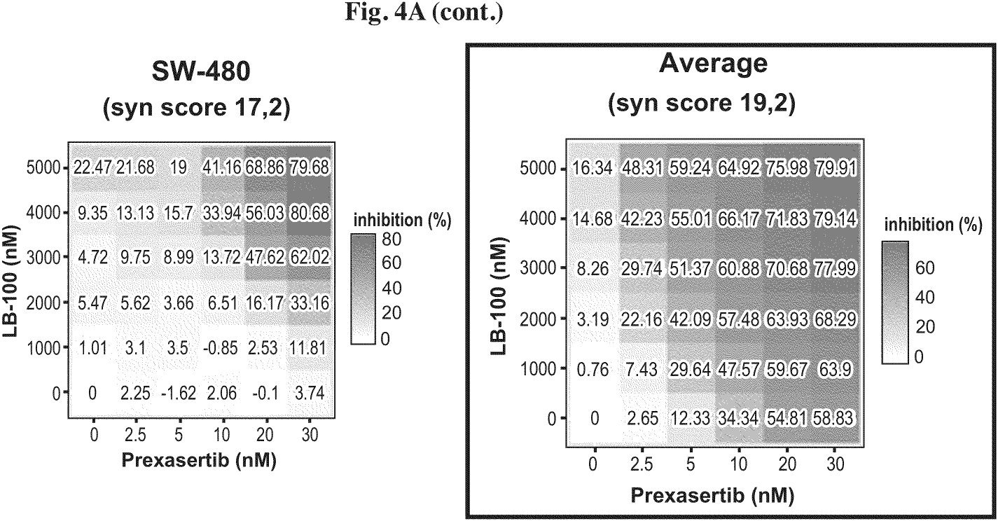

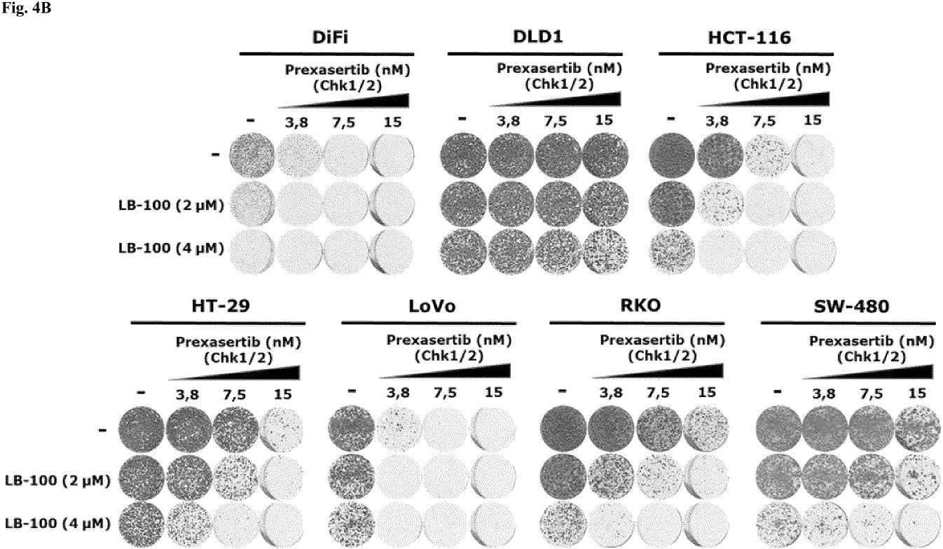

A similar synergy experiment was carried out by combining LB-100 with prexasertib. Across each of the matrices, the results consistently showed that LB-100 also synergizes with prexasertib in the seven colorectal cancer cell lines, with an average synergy score of 19.2 (FIG. 4A). The toxicity of this combination across the panel was also further confirmed in long-term cell proliferation assays (FIG. 4B).

### Example 4. Identification of Stress-Targeted Drugs That in Combination with LB-100 Kill RBE Cholangiocarcinoma Cells

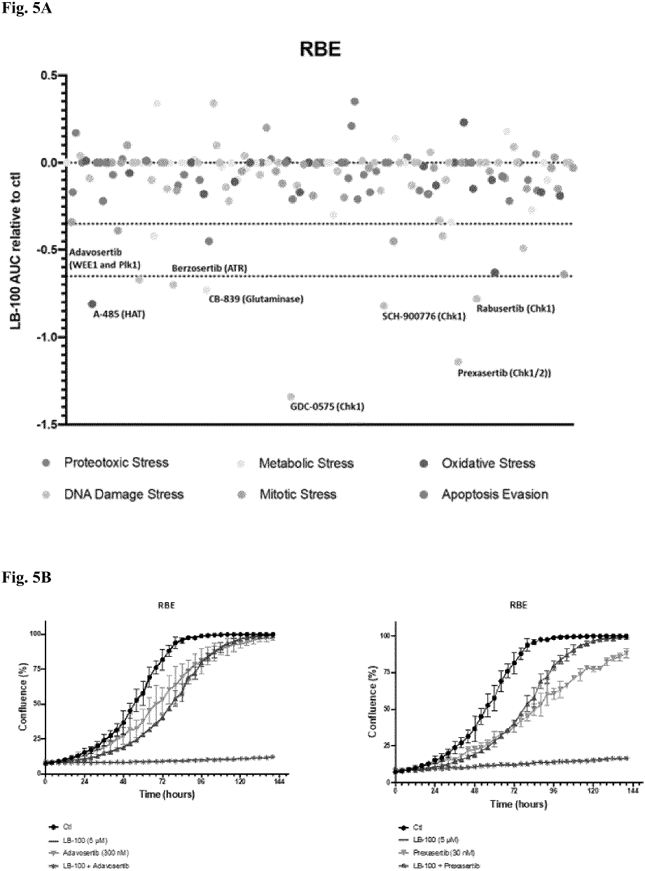

To evaluate whether LB-100 promotes similar sensitization to stress-targeted drugs in a different cancer cell line, a similar stress-focused drug screen was performed in RBE cholangiocarcinoma cells. As shown in FIG. 5A, the CHK1 inhibitors GDC-0575, prexasertib, rabusertib, and SCH-900776; the WEE1 kinase inhibitor adavosertib; and the ATR inhibitor berzosertib, were among the top 5% in increased drug efficacy as measured by the normalized AUCs of the screened compounds.

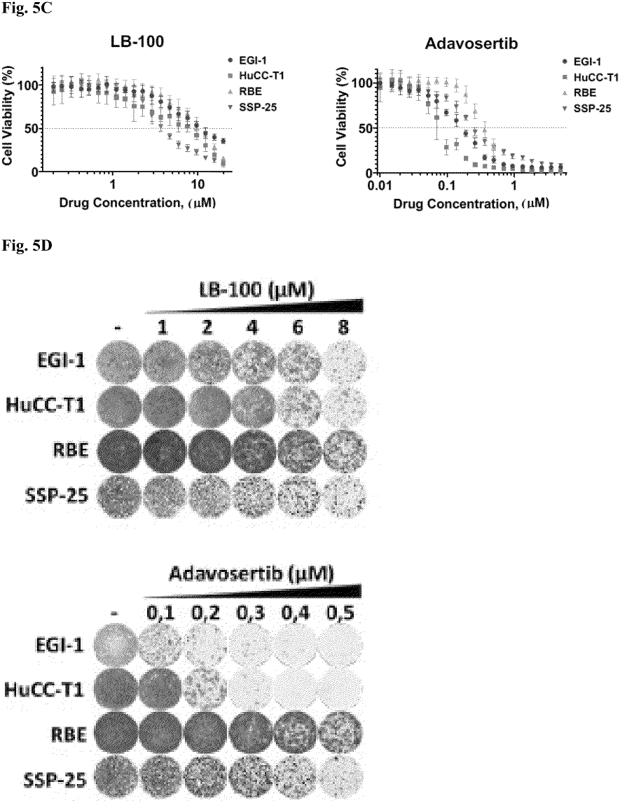

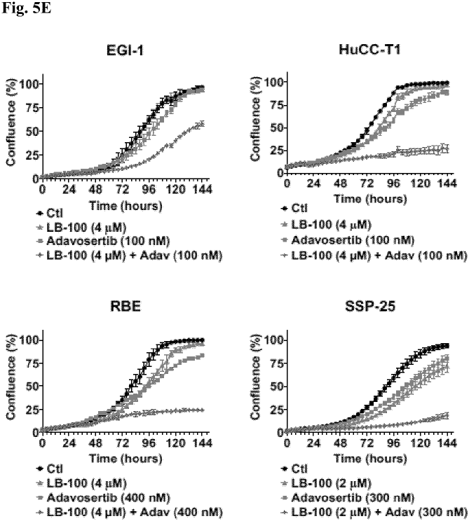

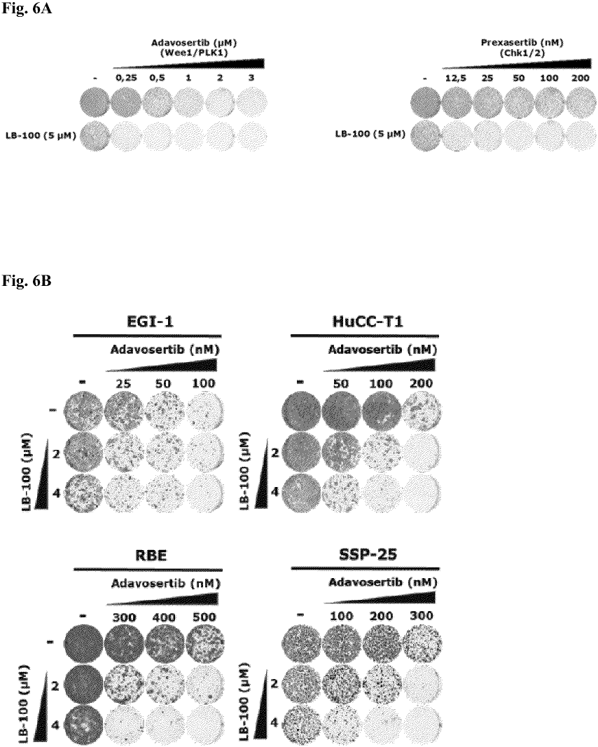

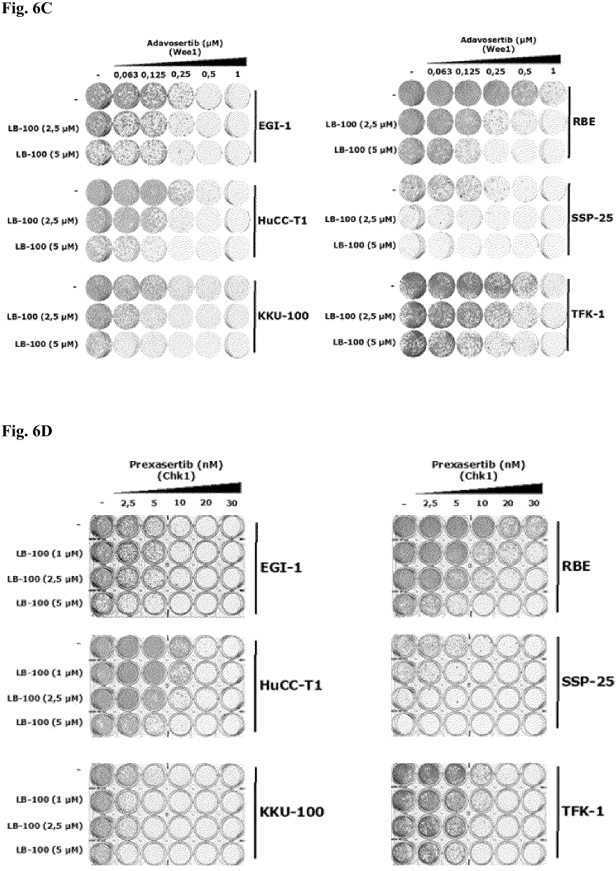

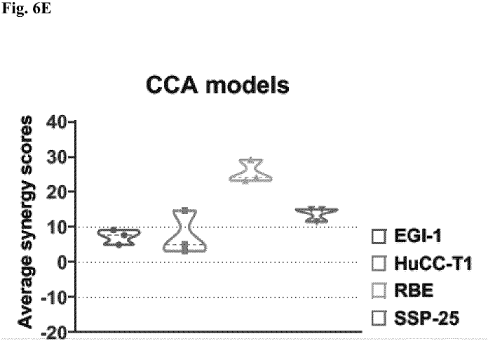

RBE cells were plated and incubated overnight to allow attachment to the plate. Cells were then treated at the indicated conditions and toxicity was measured by IncuCyte® as shown by the proliferation curves provided in FIG. 5B. Longer-term toxicity was also evaluated. For these experiments, cells were grown in the absence or presence of LB-100 and adavosertib or prexasertib, at the indicated concentrations for 7 days, then fixed and stained (FIG. 6A). The results provided in FIG. 5B and FIG. 6A confirm the toxicity of the combinations of LB-100 with adavosertib or prexasertib in RBE cholangiocarcinoma cells. The stress-focused drug screen of FIG. 5A indicates that synthetic lethality of LB-100 in combination with DNA damage checkpoint inhibition occurs in RBE cholangiocarcinoma cells.

The same drug combinations were also investigated in five additional cholangiocarcinoma cell lines under the conditions outlined above. From these studies it was found that the combinations exhibited increased toxicity compared to the single-drug treatment in all the cell lines, despite differences in sensitivity across the cell lines (FIG. 6C and FIG. 6D).

### Example 5. Synergistic Effect of LB-100 With Adavosertib and Prexasertib in Pancreatic Ductal Adenocarcinoma and Cholangiocarcinoma Cell Lines

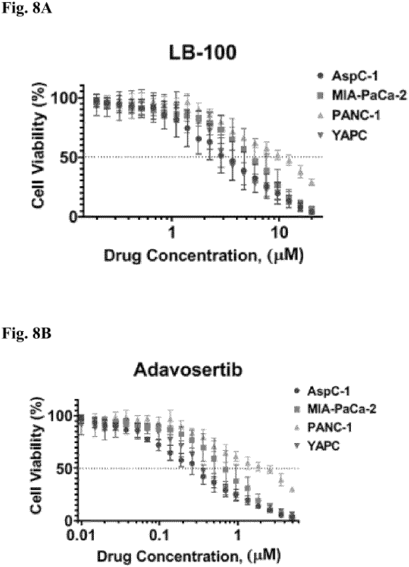

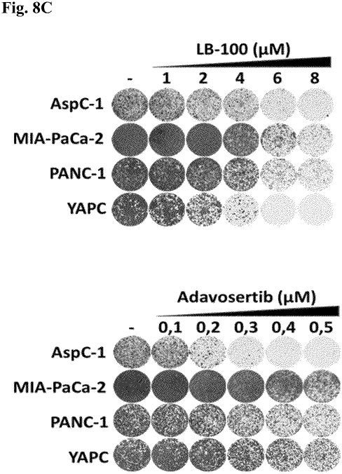

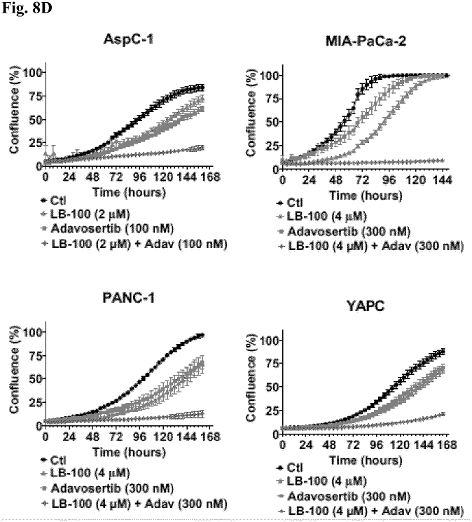

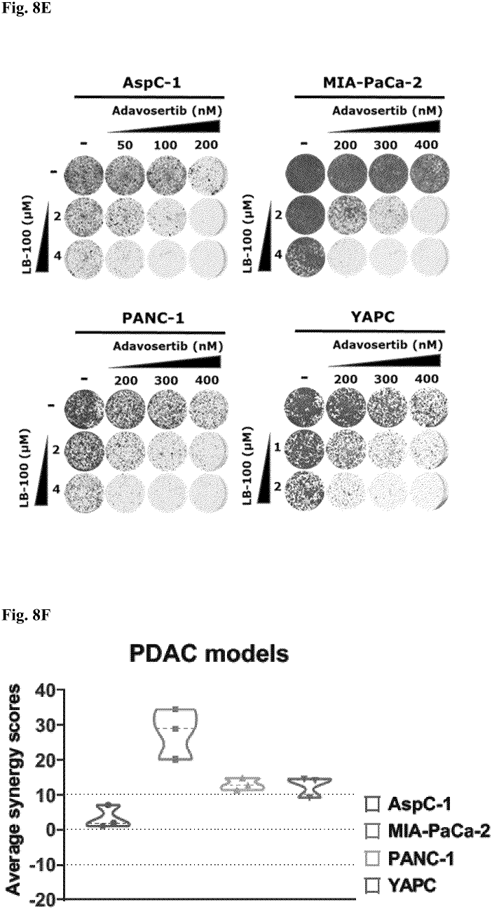

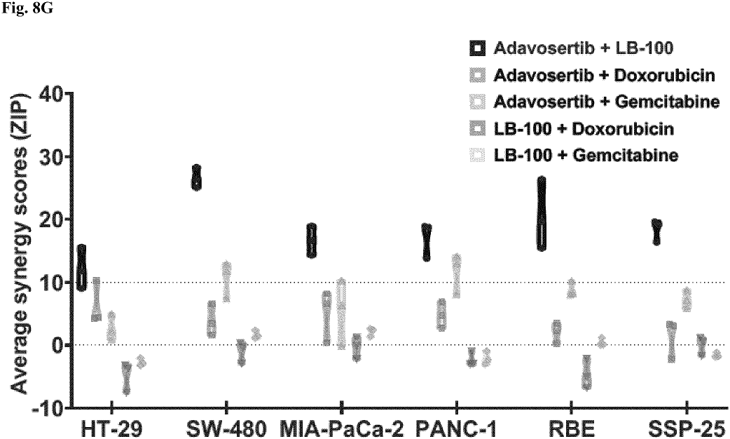

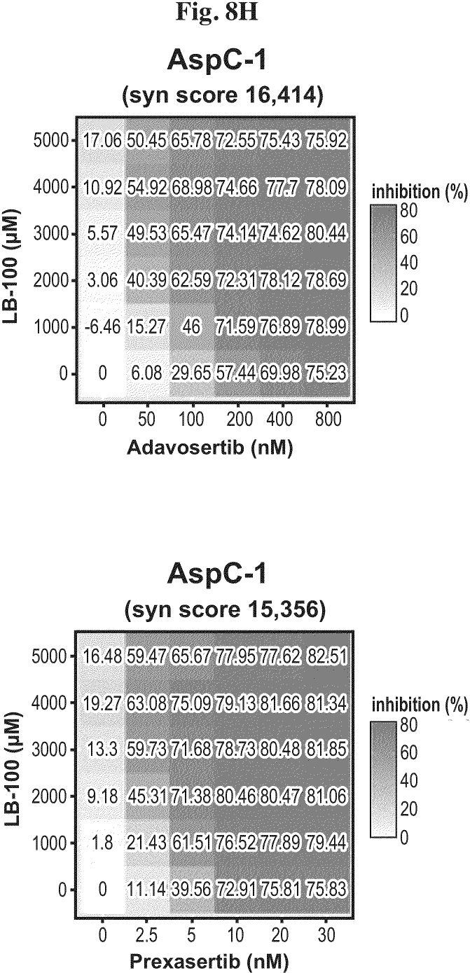

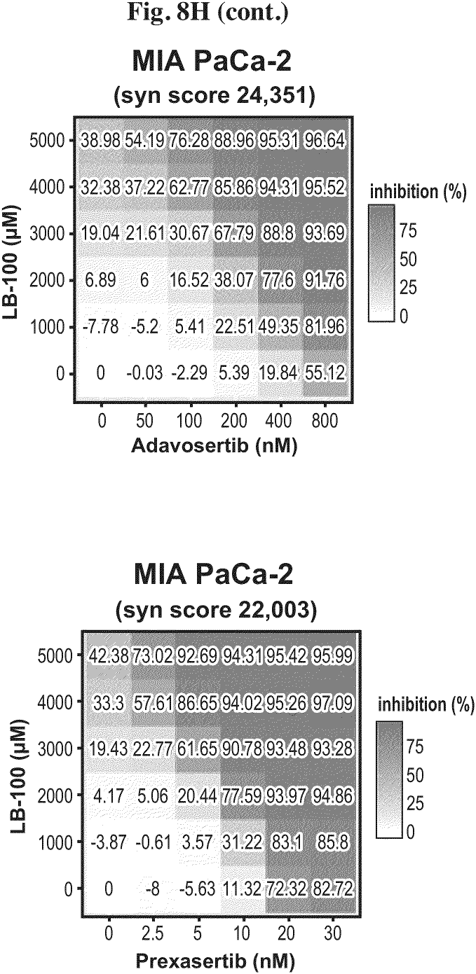

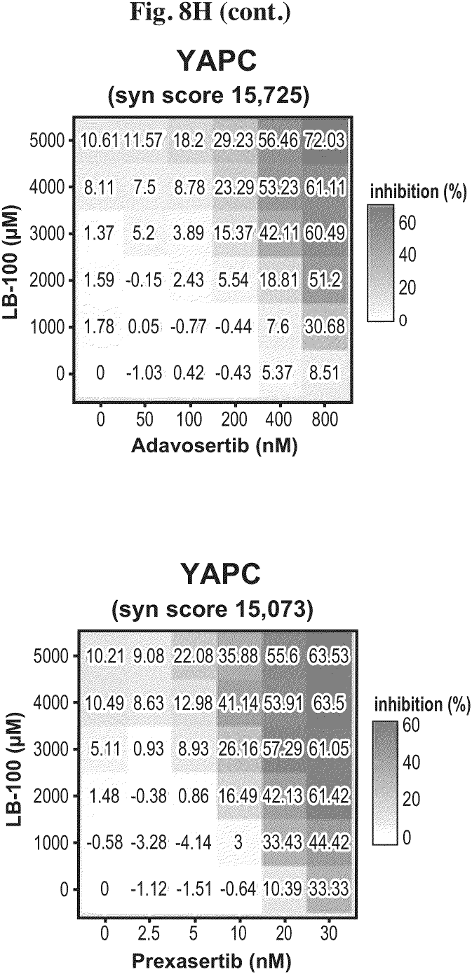

The efficacy of the LB-100/adavosertib combination in the CRC cell lines encouraged further evaluation in other tumor types lacking effective treatment options. Pancreatic ductal adenocarcinomas (PDACs) are refractory to conventional therapies and the 5-year survival rates remain one of the lowest among all cancers. Similarly, despite a much lower overall incidence, cholangiocarcinomas (CCAs) share with PDACs the frequent lack of response to conventional therapies and the dismal prognosis. A panel of four PDAC cell lines and a similar one with CCA cells were assembled to assess the efficacy of LB-100 and adavosertib in these cancer types. LB-100 dose-response curves revealed IC50s varying from 3.2 to >10 µM in the PDAC cells (FIG. 8A), and from 4.7 to >12 µM in the CCA cell lines (FIG. 5C, left panel). For adavosertib, the IC50s ranged from 0.3 to 1.7 µM in the PDAC cell lines (FIG. 8B), and from 0.1 to 0.37 µM in the CCA cell lines (FIG. 5C, right panel). To investigate the effects of the combination in the PDAC and CCA cancer cell lines systematically, the same experimental workflow used for the CRC panel was employed. First, the long-term toxicity of LB-100 and adavosertib was addressed in the PDAC and CAA cell lines (FIG. 5D and FIG. 8C). Then, sub-lethal doses of each drug were combined and showed strong or complete suppression of cell viability in the cell lines from both cancer types (FIG. 6B and FIG. 8E). Ineffective doses of individual drugs, in the absence of the other, suppress cell proliferation in combination in each of the PDAC and CCA cell lines (FIG. 5E and FIG. 8D). Furthermore, matrices of LB-100 and adavosertib combinations indicated synergy in three out of the four PDAC cell lines (FIG. 8F), AspC-1 being the exception. For the CCA panel, clear synergy was found for RBE and SSP-25, but not for EGI and HuCC-T1 cells (FIG. 6E). As observed for DiFi and RKO, the doses of adavosertib used in the synergy assays are already toxic to AspC-1, EGI, and HuCC-T1 cells (FIG. 5C, FIG. 8A, and FIG. 8B).

The combination of LB-100 and adavosertib was compared LB-100 and adavosertib each in combination with doxorubicin or gemcitabine. Synergy matrices were prepared using 10 concentrations of each of LB-100 and adavosertib in two cell lines per tumor type. The results showed higher synergy scores for the LB-100 + adavosertib combination compared to combinations with the chemotherapeutic agents in each of the cell lines (FIG. 8G).

These data reveal remarkable context independence of the synthetic lethality of LB-100 in combination with adavosertib in cancer cell lines from different tissues and diverse genetic backgrounds. This combination is believed to provide therapeutic benefits superior to combinations that are currently under clinical investigation. A mechanistic understanding of this toxicity and the evaluation of the viability of this combination in vivo were investigated.

The ability of LB-100 to synergize with prexasertib in pancreatic cancer cell lines was also investigated. In this study, AspC-1, MIA PaCa-2, and YAPC cells were cultured with the indicated concentrations of LB-100 and prexasertib for 5 days, then the cell viability was measured using resazurin. The relative inhibition of cell viability is shown for each pair of concentrations. Synergy scores (ZIP) were calculated using the SynergyFinder 2.0 online tool. A score above 10 indicates synergy. The synergy matrices shown in FIG. 8H indicate that this combination also shows synergy in various pancreatic cancer lines.

### Example 6. Anti-Proliferative Effects of LB-100 in Combination with Adavosertib or Prexasertib in Two High-Grade Ovarian Cancer Cell Lines

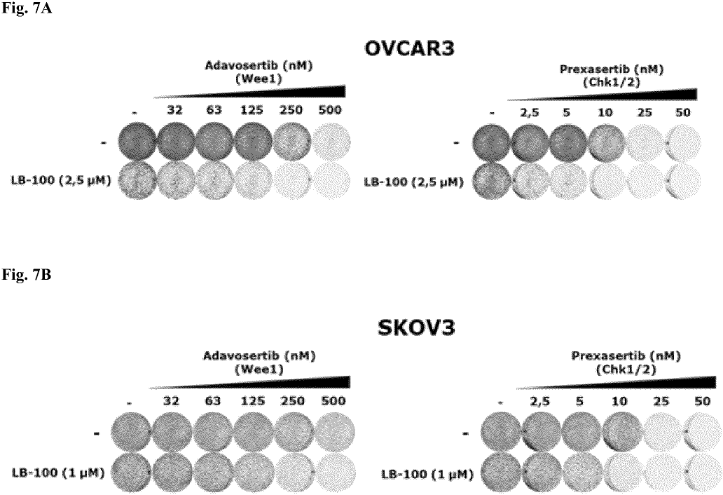

The anti-proliferative effect of LB-100 in combination with adavosertib or prexasertib was also evaluated in OVCAR3 (FIG. 7A) and SKOV3 (FIG. 7B) ovarian cancer cell lines. Cells were grown in presence of LB-100 and with or without adavosertib or prexasertib at the indicated concentrations for 7 days, then fixed and stained. Long-term proliferation assays indicate that LB-100 in combination with adavosertib or prexasertib is toxic to these cells at concentrations at which the single drugs show limited toxicity.

### Example 7. Acquired Resistance to the Combination of LB-100 and Adavosertib is Tumor-Suppressive

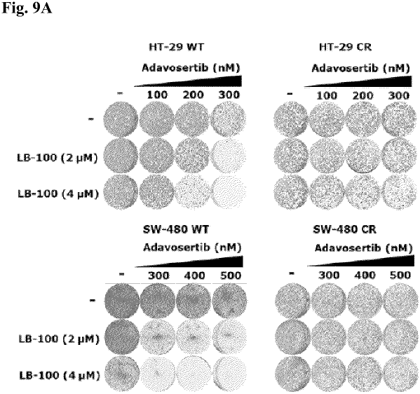

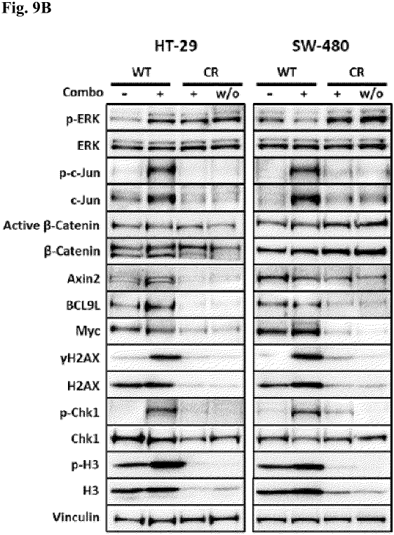

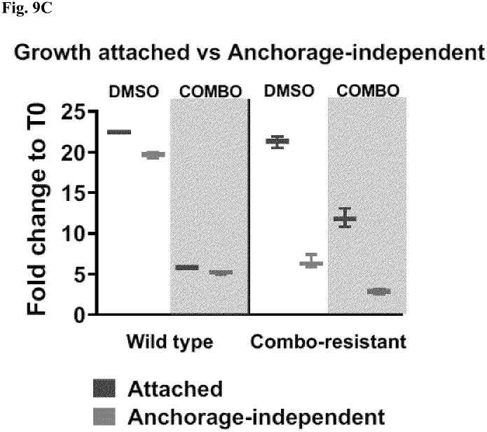

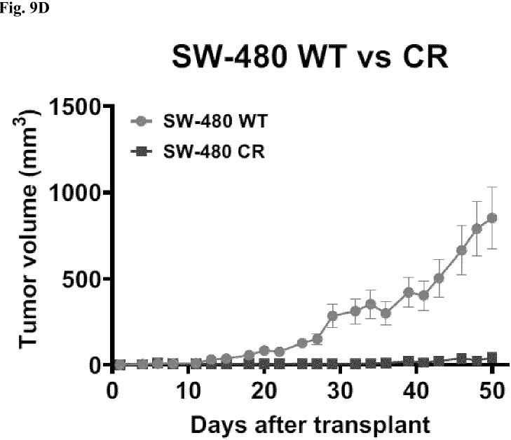

Even highly synergistic drug combinations ultimately result in resistance in patients with advanced disease. Since deliberate activation of oncogenic signaling is fundamentally different from inhibition of these signals, a study was conducted to see how cancer cells can acquire resistance to the combination of LB-100 and adavosertib. HT-29 and SW-480 resistant cells (HT-29 CR and SW-480 CR) were selected by culturing them in the presence of the drug combination for over four months. Long-term viability assays showed that despite growing in the presence of the drugs for several months, the combination of LB-100 and adavosertib still partially hindered cell viability in both cell lines. Yet, the reduced toxicity of the single drugs and the combination compared to the respective parental cells is clear (FIG. 9A).

It was reasoned that acquired resistance to hyper-activation of oncogenic pathways might develop in the opposite direction as is seen when activated pathways are inhibited and thus may lead to the down-modulation of oncogenic signaling. It was found that p-ERK levels remain higher in the combo-resistant cells compared to parental controls. Conversely, c-Jun is no longer hyperactivated in the resistant cells in the presence of drug, suggesting downmodulation of this MAPK signaling arm. Moreover, the levels of active (non-phospho) or total β-catenin are not altered in HT-29 and or SW-480 CR cells compared to the parental cell lines. However, the levels of the β-catenin targets AXIN2 and MYC, as well as the modulator of β-catenin transcriptional activity BCL9L were lower in CR cells irrespective of the presence of the drugs (FIG. 9B). Furthermore, for both CRC cell lines p-CHK1, γ-H2AX, and p-H3 (Ser10) levels are no longer increased in the presence of the drug (FIG. 9B).

The data above showing reduced oncogenic signaling output after acquired resistance show an unexpected outcome for the combination of LB-100 and adavosertib: loss of the oncogenic phenotype as a result of drug resistance. Anchorage-independent proliferation is a common trait of transformed cells and can be used as a proxy for oncogenic potential. How acquired resistance to this combination would modulate anchorage-independent proliferation in these CRC cell lines was considered. Parental SW-480 cells showed similar endpoint viability both in attached or anchorage-independent conditions. The addition of this drug combination also similarly restrained cell viability under both conditions (FIG. 9C). The proliferation of attached SW-480 CR cells was like that of parental cells. Strikingly, a stark decrease in cell proliferation was observed under anchorage-independent conditions, even in the absence of the drugs. The addition of the combination further reduced cell viability under both conditions (FIG. 9C). Similar results were observed in HT-29 cells.

SW-480 parental and CR cells were transplanted into immunocompromised mice, and tumor growth was monitored for 2 months. The results showed clear engraftment within the first 25 days and steady tumor growth in mice transplanted with SW-480 parental cells. Conversely, SW-480 CR cells either failed to develop tumors or developed tumors only over 50 days after transplantation (FIG. 9D).

Altogether, the data from the stress-focused drug screens, the genome-wide CRISPR screen, and the validations across colorectal, cholangiocarcinoma, high-grade ovarian cancer and pancreatic ductal adenocarcinoma cell lines indicate that combining (a) LB-100 and (b) a WEE1 or CHK1 inhibitor kills cancer cells from different tissues and genetic backgrounds.

What is claimed is:

1. A method for treating cancer, comprising administering to a subject in need thereof an effective amount of:
(a) LB-100 or a pharmaceutically acceptable salt thereof; and
(b) a WEE1 kinase inhibitor, checkpoint kinase 1 (“CHK1”) inhibitor, or B-cell lymphoma-extra large (“BCL-xL”) inhibitor, wherein the cancer is hepatocellular carcinoma (hepatoma), cholangiocarcinoma, colorectal carcinoma, small cell lung cancer, non-small cell lung cancer, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing’s tumor, leiomyosarcoma, rhabdomyosarcoma, pancreatic cancer, breast cancer, triple negative breast cancer, ovarian cancer, endometrial carcinoma, Fallopian tube cancer, prostate cancer, gastrointestinal stromal tumor (GIST), esophageal cancer, gallbladder cancer, gastrointestinal carcinoid tumor, duodenal cancer, gastroesophageal junction cancer, islet cell cancer, gastric cancer, anal cancer, cancer of the small intestine, pseudomyxoma peritonei, head and neck squamous cell carcinoma, Merkel cell carcinoma, tumor mutational burden-high cancer (TMB-H), microsatellite stable (MSS), mismatch repair proficient colon cancer, thyroid cancer, renal cell carcinoma, choriocarcinoma, seminoma, embryonal carcinoma, Wilms’ tumor, urothelial carcinoma, testicular cancer, bladder carcinoma, glioma, glioblastoma, multiforme, astrocytoma, medulloblastoma, craniopharyngioma, oligodendroglioma, malignant meningioma, diffuse intrinsic pontine glioma, melanoma, neuroblastoma, retinoblastoma, acute lymphoblastic B-cell leukemia, acute lymphoblastic T-cell leukemia, acute myeloblastic leukemia (AML), acute promyelocytic leukemia (APL), acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia (CML), chronic lymphocytic leukemia (CLL), hairy cell leukemia, multiple myeloma, lymphoblastic leukemia, myelogenous leukemia, lymphocytic leukemia, Hodgkin’s lymphoma, non-Hodgkin’s lymphoma, primary mediastinal large B-cell lymphoma, Waldenstrom’s macroglobulinemia, heavy chain disease, or polycythemia vera.

2-4. (canceled)

5. The method of claim 1, wherein the LB-100 or pharmaceutically acceptable salt thereof is administered intravenously.

6. The method of claim 5, wherein the LB-100 or pharmaceutically acceptable salt thereof is administered to the subject at a dose of about 0.25 mg/m² to about 3.1 mg/m².

7. (canceled)

8. The method of claim 1, wherein the LB-100 or pharmaceutically acceptable salt thereof is administered to the subject daily for 3 consecutive days every 3 weeks.

9. (canceled)

10. The method of claim 1, comprising administering an effective amount of a WEE1 kinase inhibitor, wherein the WEE1 kinase inhibitor is adavosertib, PD0166285, PD407824, ZN-c3, IMP7068, Debio 0123, SDGR2, SY4835, or NUV-569.

11-35. (canceled)

36. The method of claim 10, wherein the WEE1 kinase inhibitor is adavosertib.

37. The method of claim 10, wherein the WEE1 kinase inhibitor is PD0166285.

38. The method of claim 1, comprising administering an effective amount of a CHK1 inhibitor, wherein the CHK1 inhibitor is GDC-0575, prexasertib, rabusertib, SCH-900776, CCT-245737, AZD-7762, PF-477736, GDC-0425, or SRA737.

39-53. (canceled)

54. The method of claim 1, comprising administering to the subject an effective amount of a BCL-xL inhibitor, wherein the BCL-xL inhibitor is A-1155463.

55-61. (canceled)

62. The method of claim 1, wherein the WEE1 kinase inhibitor, CHK1 inhibitor, or BCL-xL inhibitor is administered concurrently with, prior to, or after administering the LB-100 or pharmaceutically acceptable salt thereof.

63-74. (canceled)

75. The method of claim 1, comprising administering to the subject LB-100.

76. A composition comprising:
(1) an effective amount of:
(a) LB-100 or a pharmaceutically acceptable salt thereof; and
(b) a WEE1 kinase inhibitor, checkpoint kinase 1 (“CHK1”) inhibitor, or B-cell lymphoma-extra large (“BCL-xL”) inhibitor ; and
(2) a pharmaceutically acceptable carrier or vehicle.

77. (canceled)

78. (canceled)

79. The composition of claim 76, wherein the composition comprises a WEE1 kinase inhibitor, wherein the WEE1 kinase inhibitor is adavosertib, PD0166285, PD407824, ZN-c3, IMP7068, Debio 0123, SDGR2, SY4835, or NUV-569.

80-90. (canceled)

91. The composition of claim 76, wherein the composition comprises a CHK1 inhibitor, wherein the CHK1 inhibitor is GDC-0575, prexasertib, rabusertib, SCH-900776, CCT-245737, AZD-7762, PF-477736, GDC-0425, or SRA737.

92-100. (canceled)

101. A method for preventing, inhibiting, or reducing risk of metastasis of a cancer, comprising administering to a subject in need thereof an effect amount of:
(a) LB-100 or a pharmaceutically acceptable salt thereof; and
(b) a WEE1 kinase inhibitor, checkpoint kinase 1 (“CHK1”) inhibitor, or B-cell lymphoma-extra large (“BCL-xL”) inhibitor, wherein the cancer is hepatocellular carcinoma (hepatoma), cholangiocarcinoma, colorectal carcinoma, small cell lung cancer, non-small cell lung cancer, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing’s tumor, leiomyosarcoma, rhabdomyosarcoma, pancreatic cancer, breast cancer, triple negative breast cancer, ovarian cancer, endometrial carcinoma, Fallopian tube cancer, prostate cancer, gastrointestinal stromal tumor (GIST), esophageal cancer, gallbladder cancer, gastrointestinal carcinoid tumor, duodenal cancer, gastroesophageal junction cancer, islet cell cancer, gastric cancer, anal cancer, cancer of the small intestine, pseudomyxoma peritonei, head and neck squamous cell carcinoma, Merkel cell carcinoma, tumor mutational burden-high cancer (TMB-H), microsatellite stable (MSS), mismatch repair proficient colon cancer, thyroid cancer, renal cell carcinoma, choriocarcinoma, seminoma, embryonal carcinoma, Wilms’ tumor, urothelial carcinoma, testicular cancer, bladder carcinoma, glioma, glioblastoma, multiforme, astrocytoma, medulloblastoma, craniopharyngioma, oligodendroglioma, malignant meningioma, diffuse intrinsic pontine glioma, melanoma, neuroblastoma, retinoblastoma, acute lymphoblastic B-cell leukemia, acute lymphoblastic T-cell leukemia, acute myeloblastic leukemia (AML), acute promyelocytic leukemia (APL), acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia (CML), chronic lymphocytic leukemia (CLL), hairy cell leukemia, multiple myeloma, lymphoblastic leukemia, myelogenous leukemia, lymphocytic leukemia, Hodgkin’s lymphoma, non-Hodgkin’s lymphoma, primary mediastinal large B-cell lymphoma, Waldenstrom’s macroglobulinemia, heavy chain disease, or polycythemia vera.

102-104. (canceled)

105. The method of claim 101, wherein the LB-100 or pharmaceutically acceptable salt thereof is administered intravenously.

106. The method of claim 105, wherein the LB-100 or pharmaceutically acceptable salt thereof is administered to the subject at a dose of about 0.25 mg/m² to about 3.1 mg/m².

107. (canceled)

108. The method of claim 101, wherein the LB-100 or pharmaceutically acceptable salt thereof is administered to the subject daily for 3 consecutive days every 3 weeks.

109. (canceled)

110. The method of claim 101, comprising administering an effective amount of a WEE1 kinase inhibitor, wherein the WEE1 kinase inhibitor is adavosertib, PD0166285, PD407824, ZN-c3, IMP7068, Debio 0123, SDGR2, SY4835, or NUV-569.

111-135. (canceled)

136. The method of claim 110, wherein the WEE1 kinase inhibitor is adavosertib.

137. The method of claim 110, wherein the WEE1 kinase inhibitor is PD0166285.

138. The method of claim 101, comprising administering an effective amount of a CHK1 inhibitor, wherein the CHK1 inhibitor is GDC-0575, prexasertib, rabusertib, SCH-900776, CCT-245737, AZD-7762, PF-477736, GDC-0425, or SRA737.

139-153. (canceled)

154. The method of claim 101, comprising administering to the subject an effective amount of a BCL-xL inhibitor, wherein the BCL-xL inhibitor is A-1155463.

155-161. (canceled)

162. The method of claim 101, wherein the WEE1 kinase inhibitor, CHK1 inhibitor, or BCL-xL inhibitor is administered concurrently with, prior to, or after administering the LB-100 or pharmaceutically acceptable salt thereof.

163-172. (canceled)

173. The method of claim 101, comprising administering to the subject LB-100.

174-176. (canceled)
